SEQUENCE LISTING (62 sequences)

SEQ ID NO: 1 - protein, 264 aa, Unknown
  Description of Unknown Neomycin phosphotransferase protein sequence
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRYQDIALATRDIAEELGGEWADRFLVLYGIAAPDSQRIAFYRLLDEFF

SEQ ID NO: 2 - DNA, 6963 bp, Artificial Sequence
  Description of Artificial Sequence Synthetic polynucleotide
taactataacggtcctaaggtagcgaacctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaattcagtcgataactataacggtcctaaggtagcgatttaaatacgcgctctcttaaggtagccgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgctcatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggcgcgccgccaccatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtagtctagagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcgcggccgcattctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgacctagcgggcagtgagcgcaacgcaattaatgtgagttagctcactcattaggcaccccaggctttacactttatgcttccggctcgtatgttgtgtggaattgtgagcggataacaatttcacacaggaaacagctgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctagggataacagggtaatggcgcgggccgcaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggtggcaaacagctattatgggtattatgggtgacgtcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcacatgt

SEQ ID NO: 3 - DNA, 1188 bp, Artificial Sequence
  Description of Artificial Sequence Synthetic polynucleotide
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgctcatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 4 - DNA, 711 bp, Artificial Sequence
  Description of Artificial Sequence Synthetic polynucleotide
atggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagtag

SEQ ID NO: 5 - DNA, 225 bp, Artificial Sequence
  Description of Artificial Sequence Synthetic polynucleotide
gatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatc

SEQ ID NO: 6 - DNA, 795 bp, Unknown
  Description of Unknown Neomycin phosphotransferase DNA sequence
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 7 - DNA, 400 bp, Artificial Sequence
  Description of Artificial Sequence Synthetic polynucleotide
attctaccgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctc

SEQ ID NO: 8 - DNA, 96 bp, Artificial Sequence
  Description of Artificial Sequence Synthetic oligonucleotide
agcgggcagtgagcgcaacgcaattaatgtgagttagctcactcattaggcaccccaggctttacactttatgcttccggctcgtatgttgtgtgg

SEQ ID NO: 9 - DNA, 272 bp, Artificial Sequence
  Description of Artificial Sequence Synthetic polynucleotide
gggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcc

SEQ ID NO: 10 - DNA, 1119 bp, Artificial Sequence
  Description of Artificial Sequence Synthetic polynucleotide
acccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatc

SEQ ID NO: 11 - DNA, 615 bp, Artificial Sequence
  Description of Artificial Sequence Synthetic polynucleotide
cgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatccttt

SEQ ID NO: 12 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgaccctgggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 13 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggcggcctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 14 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttaacgagttcttctga

SEQ ID NO: 15 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgaccctgggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttaacgagttcttctga

SEQ ID NO: 16 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggcggcctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttaacgagttcttctga

SEQ ID NO: 17 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcggcgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttaacgagttcttctga

SEQ ID NO: 18 - protein, 307 aa, Unknown
  Description of Unknown APH(6)-Ia amino acid sequence
MSSSDHIHVPDGLAESYSRSGGEEGRAWIAGLPALVARCVDRWELKRDGGVRSGEASLVVPVLRADGTRAALKLQMPREETTAALIGLRAWGGDGMVRLLDHDEESSTMLLERLDGSRTLASVEDDDEAMGVLAGLLNRLHSVPAPPGLRGLGEIAGAMVEEVPSAVDSLADPEDRSRLRGWASAVAELVGEPGDRVLHWDLHYENVLAAEREPWLAIDPEPLVGDPGFDLWPALDTGWERIEATGDARRVVRRRFDLLTESLELDRGRAAGWTLARLLQNTLWDIEDGLTAIAPSQIAVAEALAKP

SEQ ID NO: 19 - protein, 307 aa, Unknown
  Description of Unknown APH(6)-Ib amino acid sequence
MSTSKLVEIPEPLAASYARAFGEEGQAWIAALPALVEELLDRWELTADGASASGEASLVLPVLRTDGTRAVLKLQLPREETSAAITGLRTWNGHGVVRLLDHDPRSSTMLLERLDASRTLASVEDDDAAMGVLAGLLARLVSVPAPRGLRGLGDIAGAMLEEVPRAVAALADPADRRLLNDWASAVAELVGEPGDRMLHWDLHYGNVLAAEREPWLAIDPEPLAGDPGFDLWPALDSRWDDIVAQRDVVRVVRRRFDLLTEVLGLDRARAAGWTYGRLLQNALWDIEDGSAALDPAAVTLAQALRGH

SEQ ID NO: 20 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcgggccgctatcaggacatagcgttggctacccgtgatattgctgaagagcttggcggcgaatgggctgaccgcttcctcgtgctttacggtatcgccgctcccgattcgcagcgcatcgccttctatcgccttcttaacgagttcttctga

SEQ ID NO: 21 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctgagttcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 22 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcggctgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 23 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcgggccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 24 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtggcattgctgaagagcttggcggcgaatgggctgaccgcttcctcgtgctttacggtatcgccgctcccgattcgcagcgcatcgccttctatcgccttcttaacgagttcttctga

SEQ ID NO: 25 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgcgatcaggacatagcgttggctacccgtgatattgctgaagagcttggcggcgaatgggctgaccgcttcctcgtgctttacggtatcgccgctcccgattcgcagcgcatcgccttctatcgccttcttaacgagttcttctga

SEQ ID NO: 26 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgaccagcggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttaacgagttcttctga

SEQ ID NO: 27 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttaacgagttcttctga

SEQ ID NO: 28 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtgcccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 29 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgcagccaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 30 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctttcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 31 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccatgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 32 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccatgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtgcccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 33 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccatgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgcagccaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 34 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccatgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctttcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 35 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccatgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgaccagcggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 36 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccatgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgatgatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 37 - DNA, 795 bp, Artificial Sequence
  Description of Artificial Sequence Synthetic polynucleotide
atgattgaacaagatggattgcacgcaggttctccggccgcttgggtggagaggctattcggctatgactgggcacaacagacaatcggctgctctgatgccgccatgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcgggccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctga

SEQ ID NO: 38 - protein, 264 aa, Artificial Sequence
  Description of Artificial Sequence Synthetic polypeptide
MIEQDGLHAGSPAAWVERLFGYDWAQQTIGCSDAAMFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCARLGVADRYQDIALATRDIAEELGGEWADRFLVLYGIAAPDSQRIAFYRLLDEFF

SEQ ID NO: 39 - protein, 264 aa, Artificial Sequence
  Description of Artificial Sequence Synthetic polypeptide
MIEQDGLHAGSPAAWVERLFGYDWAQQTIGCSDAAMFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGDDLVVTHGDACLPNIMVENGRFSGFIDCGRLGVADRYQDIALATRDIAEELGGEWADRFLVLYGIAAPDSQRIAFYRLLDEFF

SEQ ID NO: 40 - protein, 264 aa, Artificial Sequence
  Description of Artificial Sequence Synthetic polypeptide
MIEQDGLHAGSPAAWVERLFGYDWAQQTIGCSDAAMFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRFQDIALATRDIAEELGGEWADRFLVLYGIAAPDSQRIAFYRLLDEFF

SEQ ID NO: 41 - protein, 264 aa, Artificial Sequence
  Description of Artificial Sequence Synthetic polypeptide
MIEQDGLHAGSPAAWVERLFGYDWAQQTIGCSDAAVFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVAGRYQDIALATRDIAEELGGEWADRFLVLYGIAAPDSQRIAFYRLLNEFF

SEQ ID NO: 42 - protein, 264 aa, Artificial Sequence
  Description of Artificial Sequence Synthetic polypeptide
MIEQDGLHAGSPAAWVERLFGYDWAQQTIGCSDAAMFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVADRSQDIALATRDIAEELGGEWADRFLVLYGIAAPDSQRIAFYRLLDEFF

SEQ ID NO: 43 - protein, 264 aa, Artificial Sequence
  Description of Artificial Sequence Synthetic polypeptide
MIEQDGLHAGSPAAWVERLFGYDWAQQTIGCSDAAMFRLSAQGRPVLFVKTDLSGALNELQDEAARLSWLATTGVPCAAVLDVVTEAGRDWLLLGEVPGQDLLSSHLAPAEKVSIMADAMRRLHTLDPATCPFDHQAKHRIERARTRMEAGLVDQDDLDEEHQGLAPAELFARLKASMPDGEDLVVTHGDACLPNIMVENGRFSGFIDCGRLGVAGRYQDIALATRDIAEELGGEWADRFLVLYGIAAPDSQRIAFYRLLDEFF

SEQ ID NO: 44 - protein, 264 aa, Unknown [sequence omitted]

SEQ ID NO: 45 - protein, 266 aa, Unknown
  Description of Unknown APH(6)-Ic amino acid sequence
MERWRLLRDGELLTTHSSWILPVRQGDMPAMLKVARIPDEEAGYRLLTWWDGQGAARVFASAAGALLMERASGAGDLAQIAWSGQDDEACRILCDTAARLHAPRSGPPPDLHPLQEWFQPLFRLAAEHAALAPAASVARQLLAAPREVCPLHGDLHHENVLDFGDRGWLAIDPHGLLGERTFDYANIFTNPDLSDPGRPLAILPGRLEARLSIVVATTGFEPERLLRWIIAWTGLSAAWFIGDGDGEGEGAAIDLAVNAMARRLLD

SEQ ID NO: 46 - protein, 277 aa, Unknown
  Description of Unknown APH(6)-Id amino acid sequence
MFMPPVFPAHWHVSQPVLIADTFSSLVWKVSLPDGTPAIVKGLKPIEDIADELRGADYLVWRNGRGAVRLLGRENNLMLLEYAGERMLSHIVAEHGDYQATEIAAELMAKLYAASEEPLPSALLPIRDRFAALFQRARDDQNAGCQTDYVHAAIIADQMMSNASELRGLHGDLHHENIMFSSRGWLVIDPVGLVGEVGFGAANMFYDPADRDDLCLDPRRIAQMADAFSRALDVDPRRLLDQAYAYGCLSAAWNADGEEEQRDLAIAAAIKQVRQTS

SEQ ID NO: 47 - protein, 264 aa, Unknown
  Description of Unknown APH(3′)-IIIa amino acid sequence
MAKMRISPELKKLIEKYRCVKDTEGMSPAKVYKLVGENENLYLKMTDSRYKGTTYDVEREKDMMLWLEGKLPVPKVLHFERHDGWSNLLMSEADGVLCSEEYEDEQSPEKIIELYAECIRLFHSIDISDCPYTNSLDSRLAELDYLLNNDLADVDCENWEEDTPFKDPRELYDFLKTEKPEEELVFSHGDLGDSNIFVKDGKVSGFIDLGRSGRADKWYDIAFCVRSIREDIGEEQYVELFFDLLGIKPDWEKIKYYILLDELF

SEQ ID NO: 48 - protein, 250 aa, Unknown
  Description of Unknown APH(3′)-VIIa amino acid sequence
MKYIDEIQILGKCSEGMSPAEVYKCQLKNTVCYLKKIDDIFSKTTYSVKREAEMMMWLSDKLKVPDVIEYGVREHSEYLIMSELRGKHIDCFIDHPIKYIECLVNALHQLQAIDIRNCPFSSKIDVRLKELKYLLDNRIADIDVSNWEDTTEFDDPMTLYQWLCENQPQEELCLSHGDMSANFFVSHDGIYFYDLARCGVADKWLDIAFCVREIREYYPDSDYEKFFFNMLGLEPDYKKINYYILLDEMF

SEQ ID NO: 49 - protein, 259 aa, Unknown
  Description of Unknown APH(3′)-VIa amino acid sequence
MELPNIIQQFIGNSVLEPNKIGQSPSDVYSFNRNNETFFLKRSSTLYTETTYSVSREAKMLSWLSEKLKVPELIMTFQDEQFEFMITKAINAKPISALFLTDQELLAIYKEALNLLNSIAIIDCPFISNIDHRLKESKFFIDNQLLDDIDQDDFDTELWGDHKTYLSLWNELTETRVEERLVFSHGDITDSNIFIDKFNEIYFLDLGRAGLADEFVDISFVERCLREDASEETAKIFLKHLKNDRPDKRNYFLKLDELN

SEQ ID NO: 50 - protein, 262 aa, Unknown
  Description of Unknown APH(3′)-IVa amino acid sequence
MNESTRNWPEELLELLGQTELTVNKIGYSGDHVYHVKEYRGTPAFLKIAPSVWWRTLRPEIEALAWLDGKLPVPKILYTAEHGGMDYLLMEALGGKDGSHETIQAKRKLFVKLYAEGLRSVHGLDIRECPLSNGLEKKLRDAKRIVDESLVDPADIKEEYDCTPEELYGLLLESKPVTEDLVFAHGDYCAPNLIIDGEKLSGFIDLGRAGVADRYQDISLAIRSLRHDYGDDRYKALFLELYGLDGLDEDKVRYYIRLDEFF

SEQ ID NO: 51 - protein, 271 aa, Unknown
  Description of Unknown APH(3′)-Ia amino acid sequence
MSHIQRETSCSRPRLNSNMDADLYGYKWARDNVGQSGATIYRLYGKPDAPELFLKHGKGSVANDVTDEMVRLNWLTEFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 52 - protein, 271 aa, Unknown
  Description of Unknown APH(3′)-Ic amino acid sequence
MSHIQRETSCSRPRLNSNLDADLYGYRWARDNVGQSGATIYRLYGKPNAPELFLKHGKGSVANDVTDEMVRLNWLTAFMPLPTIKHFIRTPDDAWLLTTAIPGKTAFQVLEEYPDSGENIVDALAVFLRRLHSIPVCNCPFNSDRVFRLAQAQSRMNNGLVDASDFDDERNGWPVEQVWKEMHKLLPFSPDSVVTHGDFSLDNLIFDEGKLIGCIDVGRVGIADRYQDLAILWNCLGEFSPSLQKRLFQKYGIDNPDMNKLQFHLMLDEFF

SEQ ID NO: 53 - protein, 269 aa, Unknown
  Description of Unknown APH(3′)-Ib amino acid sequence
MNDIDREEPCAAAAVPESMAAHVMGYKWARDKVGQSGCAVYRLHSKSGGSDLFLKHGKDAADDVTDEMVRLRWLAGHISVPSVVSFVRTPNQAWLLTTAIHGKTAFQVLEEYPDSGENIVDAAVFLRRLHSIPVCNCPFNSDRVFRLAQAQSRMNNGLVDVDDFDKEREGWTAEQVWEAMHRLLPLAPDPVVTHGDFSLDNLLIVEGKVVGCIDVGRAGIADRYQDLAVLWNCLEEFEPSLQERLVAQYGIADPDRRKLQFHLLLDELF

SEQ ID NO: 54 - protein, 264 aa, Unknown [sequence omitted]

SEQ ID NO: 55 - protein, 263 aa, Unknown
  Description of Unknown APH(3′)-Vb amino acid sequence
MESTLRRTYPHHTWHLVNEGDSGAFVYRLTGHGPELYAKIAPRTPENSAFHLDGEADRLDWLARHGISVPRVVERGADDTTAWLVTEAVPGAASAEEWPEDERAAVVDAIAEMARTLHELPVSECPFDRRLDVTGEARHNVREGLVDLDDLQEEPAGWTGDQLLAELDLTRPEKEDLVVCHGDLCPNNVLLDPETHRVTGLIDVGRLRLATCHADIALAARELAIDEDPWFGPAYAERFLERYGAHHVDQEKMAFYQLLDEFF

SEQ ID NO: 56 - protein, 268 aa, Unknown
  Description of Unknown APH(3′)-Va amino acid sequence
MDDSTLRRKYPHHEWHAVNEGDSGAFVYQLTGGPEPQPELYAKIAPRAPENSAFDLSGEADRLEWLHRHGIPVPRVVERGADDTAAWLVTEAVPGVAAAEEWPEHQRFAVVEAMAELARALHELPVEDCPSDRRLDAAVAEARRNVAEGLVDLDDLQEERAGWTGDQLLAELDRTRPEKEDLVVCHGDLCPNNVLLDPGTCRVTGVIDVGRLGVADRHADIALAARELEIDEDPWFGPAYAERFLERYGAHRVDKEKLAFYQLLDEFF

SEQ ID NO: 57 - protein, 264 aa, Unknown
  Description of Unknown APH(3′)-Vc amino acid sequence
MYAMLRRKYQHYEWTSVNEGDSGASVYRLAGQQPELYVKFAPREPENSAFDLAGEADRLTWLTRHGIPVPCIVECGGDDTSVFLVTEAVTGVAAAEEWPEHQRFAVVEAMADLARTLHELPVGGCPFDRSLAVTVAEARHNLREGLVDLDDLQEEHANWSGDQLLAELDRTRPEKEDLVVCHGDLCPNNVLLDPETCRVTGMIDVGRLGRADRHADIALAARELEIDEDPWFGPEYAQRFLERYGAHHVDENKMAFYQLLDEFF

SEQ ID NO: 58 - protein, 272 aa, Unknown
  Description of Unknown APH(3“)-Ia amino acid sequence
MSDHPGPGAVTPELFGVGGDWLAVTAGESGASVFRAADATRYAKCVPAADAAGLEAERDRIAWLSGQGVPGPRVLDWYAGDAGACLVTRAVPGVPADRVGADDLRTAWGAVADAVRRLHEVPVASCPFRRGLDSVVDAARDVVARGAVHPEFLPVEQRLVPPAELLARLTGELARRRDQEAADTVVCHGDLCLPNIVLHPETLEVSGFIDLGRLGAADRHADLALLLANARETWVDEERARFADAAFAERYGIAPDPERLRFYLHLDPLTWG

SEQ ID NO: 59 - protein, 267 aa, Unknown
  Description of Unknown APH(3”)-Ib amino acid sequence
LNRTNIFFGESHSDWLPVRGGESGDFVFRRGDGHAFAKIAPASRRGELAGERDRLIWLKGRGVACPEVINWQEEQEGACLVITAIPGVPAADLSGADLLKAWPSMGQQLGAVHSLSVDQCPFERRLSRMFGRAVDVVSRNAVNPDFLPDEDKSTPLHDLLARVERELPVRLDQERTDMVVCHGDPCMPNFMVDPKTLQCTGLIDLGRLGTADRYADLALMIANAEENWAAPDEAERAFAVLFNVLGIEAPDRERLAFYLRLDPLTWG

SEQ ID NO: 60 - protein, 319 aa, Unknown
  Description of Unknown APH(2“)-Ia amino acid sequence
EHELHEGKKEDCYLMEYRYDDNATNVKAMKYLIEHYFDNFKVDSIEIIGSGYDSVAYLVNNEYIFKTKFSTNKKKGYAKEKAIYNFLNTNLETNVKIPNIEYSYISDELSILGYKEIKGTFLTPEIYSTMSEEEQNLLKRDIASFLRQMHGLDYTDISECTIDNKQNVLEEYILLRETIYNDLTDIEKDYIESFMERLNATTVFEGKKCLCHNDFSCNHLLLDGNNRLTGIIDFGDSGIIDEYCDFIYLLEDSEEEIGTNFGEDILRMYGNIDIEKAKEYQDIVEEYYPIETIVYGIKNIKQEFIENGRKEIYKRTYKD

SEQ ID NO: 61 - protein, 332 aa, Unknown
  Description of Unknown APH(4)-Ib amino acid sequence
MTQESLLLLDRIDSDDSYASLRNDQEFWEPLARRALEELGLPVPPVLRVPGESTNPVLVGEPDPVIKLFGEHWCGPESLASESEAYAVLADAPVPVPRLLGRGELRPGTGAWPWPYLVMSRMTGTTWRSAMDGTTDRNALLALARELGRVLGRLHRVPLTGNTVLTPHSEVFPELLRERRAATVEDHRGWGYLSPRLLDRLEDWLPDVDTLLAGREPRFVHGDLHGTNIFVDLAATEVTGIVDFTDVYAGDSRYSLYQLHLNAFRGDREILAALLDGAQWKRTEDFARELLAFTFLHDFEVFEETPLDLSGFTDPEELAQFLWGPPDTAPGA

SEQ ID NO: 62 - protein, 341 aa, Unknown
  Description of Unknown APH(4)-Ia amino acid sequence
MKKPELTATSVEKFLIEKFDSVSDLMQLSEGEESRAFSFDVGGRGYVLRVNSCADGFYKDRYVYRHFASAALPIPEVLDIGEFSESLTYCISRRAQGVTLQDLPETELPAVLQPVAEAMDAIAAADLSQTSGFGPFGPQGIGQYTTWRDFICAIADPHVYHWQTVMDDTVSASVAQALDELMLWAEDCPEVRHLVHADFGSNNVLTDNGRITAVIDWSEAMFGDSQYEVANIFFWRPWLACMEQQTRYFERRHPELAGSPRLRAYMLRIGLDQLYQSLVDGNFDDAAWAQGRCDAIVRSGAGTVGRTQIARRSAAVWTDGCVEVLADSGNRRPSTRPRAKE

(19) United States
(12) Patent Application Publication
(10) Pub. No.: US 2024/0191215 A1
(43) Pub. Date: Jun. 13, 2024

(54) MATERIALS AND METHODS FOR IMPROVED PHOSPHOTRANSFERASES

(71) Applicant: Janssen Biotech, Inc., Horsham, PA (US)

(72) Inventor: William Lloyd Perry, III, Warrington, PA (US)

(21) Appl. No.: 18/287,742

(22) PCT Filed: Apr. 20, 2022
(86) PCT No.: PCT/US2022/025452

§ 371 Date: Oct. 20, 2023

Related U.S. Application Data
(60) Provisional application No. 63/177,739, filed on Apr. 21, 2021.
(60) Provisional application No. 63/177,744, filed on Apr. 21, 2021.
(60) Provisional application No. 63/177,746, filed on Apr. 21, 2021.
(60) Provisional application No. 63/177,749, filed on Apr. 21, 2021.
(60) Provisional application No. 63/177,753, filed on Apr. 21, 2021.
(60) Provisional application No. 63/177,759, filed on Apr. 21, 2021.
(60) Provisional application No. 63/177,764, filed on Apr. 21, 2021.
(60) Provisional application No. 63/177,767, filed on Apr. 21, 2021.

(51) Int. Cl. C12N 9/12; C12Q 1/48

(52) U.S. Cl. CPC C12N 9/1205; C12Q 1/48; C12Y 207/01095

(57) ABSTRACT

Described herein are non-naturally occurring neomycin phosphotransferase (NPT) proteins and nucleic acid sequences encoding such NPT proteins. In a specific embodiment, the non-naturally occurring NPT proteins have reduced activity relative to wild-type NPT. The non-naturally occurring NPT proteins provided herein are useful as a selectable marker for screening transformed or transfected cells. Also provided herein are vectors and kits comprising a nucleic acid sequence encoding a non-naturally occurring NPT protein, and methods of producing cells expressing the non-naturally occurring NPT protein and a protein of interest or a non-coding RNA sequence of interest.

### 1. CROSS-REFERENCE TO RELATED APPLICATIONS

This application is a 371 national stage of PCT Application No. PCT/US2022/025452, filed on Apr. 20, 2022, which claims the benefit of U.S. Ser. No. 63/177,739 filed Apr. 21, 2021; U.S. Ser. No. 63/177,744 filed Apr. 21, 2021; U.S. Ser. No. 63/177,746 filed Apr. 21, 2021; U.S. Ser. No. 63/177,749 filed Apr. 21, 2021, U.S. Ser. No. 63/177,753 filed Apr. 21, 2021; U.S. Ser. No. 63/177,759 filed Apr. 21, 2021; U.S. Ser. No. 63/177,764 filed Apr. 21, 2021; U.S. Ser. No. 63/177,767 filed Apr. 21, 2021, the disclosure of each of which is incorporated by reference herein in its entirety.

### 2. SEQUENCE LISTING

This application contains a sequence listing, which is submitted electronically via EFS-Web as an ASCII formatted sequence listing with a file “14620-686-228_SL.txt” and a creation date of Apr. 9, 2022 and having a size of 118,113 bytes. The sequence listing submitted via EFS-Web is part of the specification and is herein incorporated by reference in its entirety.

### 3. FIELD

Provided herein are non-naturally occurring neomycin phosphotransferase (NPT) proteins and nucleic acid sequences encoding such NPT proteins. In a specific embodiment, the non-naturally occurring NPT proteins have reduced activity relative to wild-type NPT. The non-naturally occurring NPT proteins provided herein are useful as a selectable marker for screening transformed or transfected cells. Also provided herein are vectors and kits comprising a nucleic acid sequence encoding a non-naturally occurring NPT protein, and methods of producing cells expressing the non-naturally occurring NPT protein and a protein of interest or a non-coding RNA sequence of interest.

### 4. BACKGROUND

While it has in some instances become less difficult to generate mammalian cell lines that carry an exogenous transgene stably integrated into the genome, identifying clonal lines that express protein products at a high level and/or with high transgene copy numbers is challenging, being, for example, inefficient and time consuming, etc. Sequences at the transgene integration site can have a major effect on transgene expression (Lee et al., Trends Biotechnol., 37(9): 931-942 (2019)), resulting in dramatically different expression levels in different clones. DNA regulatory elements can be used to shield transgenes from chromosomal position effects when placed between the transgene and the host DNA (reviewed in Gupta et al., Biotechnol. Adv. 37(8): 107415 (2019)). While this approach can increase expression and expression stability, significant screening may still be needed to identify high expressing clones. For developing viral producer cell lines, it is perhaps more important to generate lines with many copies of the viral payload to be packaged than it is to have high transgene expression. A way to select for multicopy transgenes would make cell line development more efficient.

One of the problems is that many constructs used to generate stable cell lines contain very efficient selection markers that confer a selective advantage to transformed cells even when expressed at a very low level. Hence, there is no direct selection for high marker gene expression or multi-copy transgenes. Several approaches to reducing selection marker expression or translation efficiency have been described. These include using a weak promoter to drive expression (Niwa et al., Gene 108(2): 193-199 (1991); Fan et al., J Biotechnol 168(4): 652-658 (2013); Zhou et al, BMC Biotechnol. 13: 29 (2013)), initiating translation from alternate codons (e.g. GTG or TTG instead of ATG) (van Blokland et al., J Biotechnol 128(2): 237-245 (2007); Cairns et al, Biotechnol Bioeng 108(11): 2611-2622 (2011)), and using an Internal Ribosome Entry Site (IRES) to initiate translation (Gurtu et al., Biochem Biophys Res Commun 229(1): 295-298 (1996); Kwaks et al., Nat Biotechnol 21(5): 553-558 (2003); Ho et al., J Biotechnol 157(1): 130-139 (2012)).

Another approach to decreasing selection marker efficiency is to use mutant proteins with reduced activity. Mutations in the glutamine synthetase (GS) gene have been used to increase the selection stringency in CHO cells (Lin et al., MAbs 11(5): 965-976 (2019)). Neomycin phosphotransferase (NPT) from Tn5 (aminoglycoside phosphotransferase3′-IIa) is one of the most commonly used selection markers. It confers resistance to neomycin and kanamycin in bacteria and to G418 in mammalian and plant cells by phosphorylating these antibiotics (Shaw et al., Microbiol Rev 57(1): 138-163 (1993)). Mutagenesis studies (Blazquez et al., Mol. Microbiol. 5(6): 1511-1518 (1991); Kocabiyik et al., SAAS Bull Biochem Biotechnol 5: 58-63 (1992); Kocabiyik and Perlin, Biochem Biophys Res Commun 185(3): 925-931 (1992); Kocabivik and Perlin, Int J Biochem 26(1): 61-66 (1994)) and discovery of a spontaneous mutation (Yenofsky et al. Proc Natl Acad Sci USA 87(9): 3435-3439 (1990)) have identified key residues that decreased but not eliminate the ability to confer antibiotic resistance in bacteria. When mutant NPT genes were incorporated into vectors used for selecting stable antibody-producing cell lines in CHO cells, the increased stringency of selection resulted in higher antibody expression and productivity relative to the use of wild type NPT gene (Sautter and Enenkel, Biotechnol Bioeng 89(5): 530-538 (2005); Ho et al., J Biotechnol 157(1): 130-139 (2012)). Using a 2 vector system, NPT mutants with 2-16% enzyme activity increased the specific antibody productivity 5 to 10-fold relative to pools selected with the wild type NPT gene (Sautter and Enenkel 2005). When a mutant NPT gene with 3% activity was used in a single tricistronic vector, specific productivity increased 17-fold relative to use of a wild-type NPT gene (Ho et al. 2012). However, these approaches are limited.

### 5. SUMMARY

The present invention recognizes and addresses identification of NPT mutants with significantly reduced activity that would make selection of transformed cells more stringent and thereby reduce the screening necessary to identify and create cell lines expressing high levels of a transgene of interest. In one aspect, provided herein is a non-naturally occurring neomycin phosphotransferase (NPT) with neomycin phosphotransferase activity, wherein the non-naturally occurring NPT comprises one, two or more amino substitutions in wild-type NPT (e.g., one, two or more of the amino acid substitutions disclosed in Table 1 or Table 2, or a combination thereof). In certain embodiments, provided herein is a non-naturally occurring neomycin phosphotransferase (NPT) with neomycin phosphotransferase activity, wherein the non-naturally occurring NPT comprises an amino acid sequence of a wild-type neomycin phosphotransferase with amino substitutions: (a) at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (b) at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (c) at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (d) at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (e) at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (f) at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In some embodiments, the non-naturally occurring NPT comprises the amino acid sequence of SEQ ID NO:1 with amino acid substitutions: (a) at positions 36 and 210 of SEQ ID NO:1, wherein the amino acid substitution at position 36 of SEQ ID NO:1 is a substitution to methionine and the amino acid substitution at position 210 of SEQ ID NO:1 is a substitution to alanine; (b) at positions 36 and 182 of SEQ ID NO:1, wherein the amino acid substitution at position 36 of SEQ ID NO:1 is a substitution to methionine and the amino acid substitution at position 182 of SEQ ID NO:1 is a substitution to aspartic acid; (c) at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to phenylalanine; (d) amino acid substitutions at positions 216 and 261 of SEQ ID NO:1, wherein the amino acid substitution position 216 of SEQ ID NO:1 is a substitution to glycine and the amino acid substitution at position 261 of SEQ ID NO:1 is a substitution to asparagine; (e) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to serine; or (f) amino acid substitutions at positions 36 and 216 of SEQ ID NO:1, wherein the amino acid substitution at position 36 of SEQ ID NO:1 is a substitution to methionine and the amino acid substitution at position 216 of SEQ ID NO:1 is a substitution to glycine.

In some embodiments, the non-naturally occurring NPT has attenuated neomycin phosphotransferase activity as a selectivity marker as compared to wild-type NPT.

In some embodiments, the wild-type NPT comprises an amino acid sequence that is at least 80%, at least 90%, or at least 98% identical to SEQ ID NO:1. In some embodiments, the wild-type NPT comprises an amino acid sequence that is at least 60%, at least 65%, at least 70% or at least 75% identical to SEQ ID NO:1.

In some embodiments, the non-naturally occurring NPT has attenuated neomycin phosphotransferase activity as a selectivity marker as compared to wild-type NPT comprising the amino acid sequence of SEQ ID NO:1.

In some embodiments, bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT. In some embodiments, the bacterial cells are E. coli. In some embodiments, the wild-type NPT comprises the amino acid sequence of SEQ ID NO:1.

In some embodiments, mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells. In some embodiments, G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT. In certain embodiments, G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT. In some embodiments, the wild-type NPT comprises the amino acid sequence of SEQ ID NO:1. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells.

In some embodiments, the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.

In some embodiments, the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.

In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine.

In some embodiments, the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.

In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine.

In some embodiments, the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In some embodiments, the non-naturally occurring NPT comprises the amino acid sequence of SEQ ID NO:38. In some embodiments, the non-naturally occurring NPT comprises the amino acid sequence of SEQ ID NO:39. In some embodiments, the non-naturally occurring NPT comprises the amino acid sequence of SEQ ID NO:40. In some embodiments, the non-naturally occurring NPT comprises the amino acid sequence of SEQ ID NO:41. In some embodiments, the non-naturally occurring NPT comprises the amino acid sequence of SEQ ID NO:42. In some embodiments, the non-naturally occurring NPT comprises the amino acid sequence of SEQ ID NO:43.

In another aspect, provided herein is a nucleic acid comprising a first nucleotide sequence encoding the non-naturally occurring NPT as described herein. In some embodiments, the first nucleotide sequence comprises the nucleotide sequence of SEQ ID NO:20, SEQ ID NO:32, SEQ ID NO:33, SEQ ID NO:34, SEQ ID NO: 36, or SEQ ID NO:37.

In some embodiments, the nucleic acid sequence further comprises a second nucleotide sequence encoding a second protein or a non-coding RNA. In some embodiments, the second nucleotide sequence encodes a second protein and wherein the second protein is a therapeutic protein.

In another aspect, provided herein are vectors comprising the nucleic acid sequences as described herein.

In another aspect, provided herein is an in vitro or ex vivo host cell comprising the non-naturally occurring NPT. In some embodiments, the host cell comprises a nucleic acid comprising a first nucleotide sequence encoding the non-naturally occurring NPT. In some embodiments, the host cell comprises a nucleic acid comprising the nucleotide sequence of SEQ ID NO:20, SEQ ID NO:32, SEQ ID NO:33, SEQ ID NO:34, SEQ ID NO: 36, or SEQ ID NO:37. In some embodiments, the nucleic acid sequence is stably integrated into the genome of the host cell. In some embodiments, the host cell comprises a vector. In certain embodiments, the host cell is a bacterium, yeast cell, mammalian cell, or plant cell. In certain embodiments, the host cell is from a human cell line.

In another aspect, provided herein is an in vitro or ex vivo host cell expressing a non-naturally occurring NPT, wherein the non-naturally occurring NPT is attenuated relative to a wild-type neomycin phosphotransferase, and wherein the non-naturally occurring NPT comprises an amino acid sequence of the wild-type neomycin phosphotransferase with: (a) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (b) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (c) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (d) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (e) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (f) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In some embodiments, the in vitro or ex vivo host cell expresses a non-naturally occurring NPT with attenuated activity relative to a wild-type neomycin phosphotransferase, and wherein the non-naturally occurring NPT comprises the amino acid sequence of SEQ ID NO:1 with: (a) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine; (b) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid; (c) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine; (d) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine; (e) amino acid substitutions at amino acid residues 36 and 218 of SEQ ID NO:1, wherein the amino acid substitution at amino acid residue 36 of SEQ ID NO:1 is a substitution to methionine and the amino acid substitution amino acid residue 218 of SEQ ID NO:1 is a substitution to serine; or (f) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine.

In some embodiments of the in vitro or ex vivo host cell described herein, the wild-type NPT comprises an amino acid sequence that is at least 80%, at least 90%, or at least 98% identical to SEQ ID NO:1. In some embodiments, the wild-type NPT comprises an amino acid sequence that is at least 60%, at least 65%, at least 70%, or at least 75% identical to SEQ ID NO:1. In some embodiments, the non-naturally occurring NPT has attenuated neomycin phosphotransferase activity as a selectivity marker as compared to wild-type NPT comprising the amino acid sequence of SEQ ID NO:1.

In some embodiments of the in vitro or ex vivo host cell described herein, bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT. In some embodiments, the wild-type NPT comprises the amino acid sequence of SEQ ID NO:1. In some embodiments, the bacterial cells are E. coli.

In some embodiments of the in vitro or ex vivo host cell described herein, mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT. In some embodiments, G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT. In certain embodiments, G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT. In some embodiments, the wild-type NPT comprises the amino acid sequence of SEQ ID NO:1. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells.

In some embodiments of the in vitro or ex vivo host cell described herein, the expressed non-naturally occurring NPT comprises the amino acid sequence of SEQ ID NO:38 (V36M, G210A). In some embodiments, the non-naturally occurring NPT comprises the amino acid sequence of SEQ ID NO:39 (V36M, E182D). In some embodiments of the in vitro or ex vivo host cell described herein, the expressed non-naturally occurring NPT comprises the amino acid sequence of SEQ ID NO:40 (V36M, Y218F). In some embodiments of the in vitro or ex vivo host cell described herein, the expressed non-naturally occurring NPT comprises the amino acid sequence of SEQ ID NO:41 (D216G, D261N). In some embodiments of the in vitro or ex vivo host cell described herein, the expressed non-naturally occurring NPT comprises the amino acid sequence of SEQ ID NO:42 (V36M, Y218S). In some embodiments of the in vitro or ex vivo host cell described herein, the expressed non-naturally occurring NPT comprises the amino acid sequence of SEQ ID NO:43 (V36M, D216G).

In some embodiments, the in vitro or ex vivo host cell further comprises a second nucleic acid sequence encoding a second protein or a non-coding RNA. In some embodiments, the second nucleic acid sequence encodes a second protein and wherein the second protein is a therapeutic protein. In some embodiments, the second nucleic acid sequence encodes a non-coding RNA, and wherein the non-coding RNA is shRNA, miRNA, antisense RNA, guide RNA for Crispr nucleases, catalytic RNA, ribosomal RNA, or tRNA. In certain embodiments, the host cell is a bacterium, yeast cell, mammalian cell, or plant cell.

In another aspect, provided herein are methods for selecting cells with high copy numbers of a transgene and/or high expression levels of a transgene from a population of host cells in which the transgene was introduced, the method comprising: a) introducing into a population of host cells comprising a nucleic acid sequence comprising: (i) a first nucleotide sequence encoding a non-naturally occurring neomycin phosphotransferase (NPT) described herein with neomycin phosphotransferase activity; and (ii) a second nucleotide sequence comprising the transgene; and b) selecting cells that grow in the presence of a neomycin phosphotransferase substrate from the population of host cells in which the nucleic acid sequence was introduced.

In one embodiment, provided herein is a method for selecting cells with high copy numbers of a transgene and/or high expression levels of a transgene from a population of host cells in which the transgene was introduced, the method comprising: a) introducing into a population of host cells a nucleic acid sequence comprising: (i) a first nucleotide sequence encoding a non-naturally occurring neomycin phosphotransferase (NPT) with neomycin phosphotransferase activity; and (ii) a second nucleotide sequence comprising the transgene, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and b) selecting cells that grow in the presence of a neomycin phosphotransferase substrate from the population of host cells in which the nucleic acid sequence was introduced.

In certain embodiments, a method for selecting cells with high copy numbers of a transgene and/or high expression levels of a transgene from a population of host cells in which the transgene was introduced, the method comprising: a) introducing into a population of host cells a first nucleic acid sequence comprising: (i) a first nucleotide sequence encoding a non-naturally occurring neomycin phosphotransferase (NPT) with neomycin phosphotransferase activity; and (ii) a second nucleotide sequence comprising the transgene, wherein the non-naturally occurring NPT comprises the amino acid sequence of SEQ ID NO:1 with: (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine; (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid; (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine; (4) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine; (5) amino acid substitutions at amino acid residues 36 and 218 of SEQ ID NO:1, wherein the amino acid substitution at amino acid residue 36 of SEQ ID NO:1 is a substitution to methionine and the amino acid substitution amino acid residue 218 of SEQ ID NO:1 is a substitution to serine; or (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and b) selecting cells that grow in the presence of a neomycin phosphotransferase substrate from the population of host cells in which the nucleic acid sequence was introduced.

In certain embodiments of the method for selecting cells with high copy numbers of a transgene and/or high expression levels of a transgene, the non-naturally occurring NPT has attenuated neomycin phosphotransferase activity as a selectable marker as compared to wild-type NPT.

In some embodiments of the method for selecting cells with high copy numbers of a transgene and/or high expression levels of a transgene, a high copy number of a transgene is 5 to 10 times, 5 to 15 times, 2 to 5 times, 2 to 10 times, 2 to 15 times, or 10 to 20 times, 10 to 50 times, 10 to 100 times, 50 to 100 times, 50 to 200 times, 50 to 500 times, 100 to 500 times, 100 to 1000 times, 500 to 1000 times, or 2 to 1000 times higher than achieved when a nucleotide sequence encoding wild-type NPT is used in place of the non-naturally occurring NPT or mutant NPT. In some embodiments of the method for selecting cells with high copy numbers of a transgene and/or high expression levels of a transgene, a high expression level of a transgene is 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher than achieved when a nucleotide sequence encoding wild-type NPT is used in place of the non-naturally occurring NPT or mutant NPT.

In some embodiments of the method for selecting cells with high copy numbers of a transgene and/or high expression levels of a transgene, the wild-type NPT comprises an amino acid sequence that is at least 80%, at least 90%, or at least 98% identical to SEQ ID NO:1. In certain embodiments of the method for selecting cells with high copy numbers of a transgene and/or high expression levels of a transgene, the wild-type NPT comprises an amino acid sequence that is at least 60%, at least 65%, at least 70%, or at least 75% identical to SEQ ID NO:1.

In certain embodiments of the method for selecting cells with high copy numbers of a transgene and/or high expression levels of a transgene, bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT. In some embodiments, the bacterial cells are E. coli. In certain embodiments of the method for selecting cells with high copy numbers of a transgene and/or high expression levels of a transgene, mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT. In certain embodiments, G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells.

In certain embodiments of the method for selecting cells with high copy numbers of a transgene and/or high expression levels of a transgene, the non-naturally occurring NPT has attenuated neomycin phosphotransferase activity as a selectable marker as compared to wild-type NPT comprising the amino acid sequence of SEQ ID NO:1. In certain embodiments, bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT comprising the amino acid sequence of SEQ ID NO:1. In some embodiments, the bacterial cells are E. coli. In certain embodiments, mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT, wherein the wild-type NPT comprises the amino acid sequence of SEQ ID NO: 1. In certain embodiments, G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT, wherein the wild-type NPT comprises the amino acid sequence of SEQ ID NO:1. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells.

In some embodiments, the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine. In certain embodiments, the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid. In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine. In some embodiments, the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine. In certain embodiments, the non-naturally occurring NPT comprises amino acid substitutions at amino acid residues corresponding to amino acid residues 36 and 218 of SEQ ID NO:1, wherein the amino acid substitution at the amino acid residue corresponding to the amino acid residue 36 of SEQ ID NO:1 is a substitution to methionine and the amino acid substitution at the amino acid residue corresponding to amino acid residue 218 of SEQ ID NO:1 is a substitution to serine. In certain embodiments, the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In certain embodiments, the non-naturally occurring NPT comprises the amino acid sequence of SEQ ID NO:38 (V36M, G210A). In some embodiments, the non-naturally occurring NPT comprises the amino acid sequence of SEQ ID NO:39 (V36M, E182D). In certain embodiments, the non-naturally occurring NPT comprises the amino acid sequence of SEQ ID NO:40 (V36M, Y218F). In some embodiments, the non-naturally occurring NPT comprises the amino acid sequence of SEQ ID NO:41 (D216G, D261N). In certain embodiments, the non-naturally occurring NPT comprises the amino acid sequence of SEQ ID NO:42 (V36M, Y218S). In some embodiments, the non-naturally occurring NPT comprises the amino acid sequence of SEQ ID NO:43 (V36M, D216G).

In certain embodiments of the method for selecting cells with high copy numbers of a transgene and/or high expression levels of a transgene, the host cells are bacterial, yeast, mammalian or plant cells. In some embodiments, the host cells are human cells. In certain embodiments, the host cells are from a mammalian cell line (e.g., a human cell line).

In certain embodiments of the method for selecting cells with high copy numbers of a transgene and/or high expression levels of a transgene, the nucleic acid sequence is stably integrated into the genome of the selected cell. In some embodiments, the selected cells have integrated 5 to 100 copies of the transgene into their genomic DNA. In certain embodiments, the selected cells have integrated 1 to 5 copies of the transgene into their genomic DNA.

In certain embodiments of the method for selecting cells with high copy numbers of a transgene and/or high expression levels of a transgene, the selected cells have a high copy number of the transgene. In some embodiments, a high copy number of a transgene is 5 to 10 times, 5 to 15 times, 2 to 5 times, 2 to 10 times, 2 to 15 times, or 10 to 20 times, 10 to 50 times, 10 to 100 times, 50 to 100 times, 50 to 200 times, 50 to 500 times, 100 to 500 times, 100 to 1000 times, 500 to 1000 times, or 2 to 1000 times higher than achieved when a nucleotide sequence encoding wild-type NPT is used in place of the NPT mutant or non-naturally occurring NPT. In certain embodiments of the method for selecting cells with high copy numbers of a transgene and/or high expression levels of a transgene, the selected cells have high levels of expression of the transgene. In some embodiments, a high expression level of a transgene is 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher than achieved when a nucleotide sequence encoding wild-type NPT is used in place of the non-naturally occurring NPT or mutant NPT. In certain embodiments of the method for selecting cells with high copy numbers of a transgene and/or high expression levels of a transgene, the selected cells have a high copy number of the transgene and high levels of expression of the transgene. In some embodiments, a high copy number of a transgene is 5 to 10 times, 5 to 15 times, 2 to 5 times, 2 to 10 times, 2 to 15 times, or 10 to 20 times, 10 to 50 times, 10 to 100 times, 50 to 100 times, 50 to 200 times, 50 to 500 times, 100 to 500 times, 100 to 1000 times, 500 to 1000 times, or 2 to 1000 times higher than achieved when a nucleotide sequence encoding wild-type NPT is used in place of the NPT mutant or non-naturally occurring NPT. In some embodiments, a high expression level of a transgene is 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher than achieved when a nucleotide sequence encoding wild-type NPT is used in place of the non-naturally occurring NPT or mutant NPT.

In certain embodiments of the method for selecting cells with high copy numbers of a transgene and/or high expression levels of a transgene, the transgene comprises a viral gene. In some embodiments of the method for selecting cells with high copy numbers of a transgene and/or high expression levels of a transgene, the transgene comprises a human growth factor gene.

In certain embodiments of the method for selecting cells with high copy numbers of a transgene and/or high expression levels of a transgene, the neomycin phosphotransferase substrate is neomycin, kanamycin or G418.

In some embodiments of the method of selecting cells with high copy numbers of a transgene and/or high expression levels of a transgene, the selected cells comprise a 10 to 1000 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells comprise a 100 to 1000 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells comprise a 500 to 1000 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells comprise a 750 to 1000 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells comprise a 100 to 500 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells comprise a 10 to 100 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells comprise a 10 to 50 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells comprise a 10 to 25 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells comprise a 2 to 10 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene.

In some embodiments of the method of selecting cells with high copy numbers of a transgene and/or high expression levels of a transgene, the selected cells achieve a 10 to 1000 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells achieve a 100 to 1000 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells achieve a 500 to 1000 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells achieve a 750 to 1000 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells achieve a 10 to 100 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells achieve a 10 to 50 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells achieve a 5 to 25 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells achieve a 5 to 10 fold higher level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In some embodiments of the method of selecting cells with high copy numbers of a transgene and/or high expression levels of a transgene, the selected cells achieve a 2 to 10 fold higher level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene. In specific embodiments, the populations of host cells are the same and the conditions used are the same.

In certain embodiments of the method for selecting cells with high copy numbers of a transgene and/or high expression levels of a transgene, the transgene encodes a protein or a non-coding RNA. In some embodiments, the non-coding RNA is selected from the group consisting of antisense RNA, miRNA, shRNA, long non-coding RNA, catalytic RNA, ribosomal RNA, tRNA, or a guide RNA for a CRISPR nuclease. In certain embodiments, the protein is a therapeutic protein or antigen. The therapeutic protein or antigen may be one described herein or known to one of skill in the art. In certain embodiments, the protein is a viral protein. The viral protein may be one described herein or known to one of skill in the art.

In some embodiments, the use of a non-naturally occurring NPT described herein reduces the need to screen for cells transfected or transformed with a transgene by 7,500 to 10,000 fold. In some embodiments, the use of a non-naturally occurring NPT described herein reduces the need to screen for cells transfected or transformed with a transgene by 5,000 to 10,000 fold. In some embodiments, the use of a non-naturally occurring NPT described herein reduces the need to screen for cells transfected or transformed with a transgene by 2,500 to 10,000 fold. In some embodiments, the use of a non-naturally occurring NPT described herein reduces the need to screen for cells transfected or transformed with a transgene by 1,000 to 10,000 fold. In some embodiments, the use of a non-naturally occurring NPT described herein reduces the need to screen for cells transfected or transformed with a transgene by 5,000 to 7,500 fold. In some embodiments, the use of a non-naturally occurring NPT described herein reduces the need to screen for cells transfected or transformed with a transgene by 1,000 to 5,000 fold. In some embodiments, the use of a non-naturally occurring NPT described herein reduces the need to screen for cells transfected or transformed with a transgene by 500 to 1,000 fold.

In another aspect, provided herein are methods of using a plasmid or transposon comprising a nucleic acid sequence encoding a non-naturally occurring NPT described herein with attenuated neomycin phosphotransferase activity as compared to wild-type NPT as a selectable marker, the method comprising: a) introducing into a host cell a plasmid or transposon comprising the nucleic acid sequence; and b) growing the cell in the presence of a neomycin phosphotransferase substrate. In some embodiments, the methods further comprise selecting for the host cell that grows in the presence of the neomycin phosphotransferase substrate.

In one embodiment, provided herein is a method of using a plasmid or transposon comprising a nucleic acid sequence encoding a non-naturally occurring NPT with attenuated neomycin phosphotransferase activity as compared to wild-type NPT as a selectable marker, the method comprising: a) introducing into a host cell the plasmid or transposon comprising the nucleic acid sequence encoding the non-naturally occurring NPT, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and b) growing the cell in the presence of a neomycin phosphotransferase substrate. In some embodiments, the method further comprises selecting for the host cell that grows in the presence of the neomycin phosphotransferase substrate.

In some embodiments, provided herein is a method of using a plasmid or transposon comprising a nucleic acid sequence encoding a non-naturally occurring NPT with attenuated neomycin phosphotransferase activity as compared to wild-type NPT as a selectable marker, the method comprising: a) introducing into a host cell the plasmid or transposon comprising the nucleic acid sequence encoding the non-naturally occurring NPT, wherein the non-naturally occurring NPT comprises the amino acid sequence of SEQ ID NO:1 with: (1) amino acid substitutions at amino acid residues 36 and 210 of SEQ ID NO: 1, wherein the amino acid substitution at amino acid residue 36 of SEQ ID NO:1 is a substitution to methionine and the amino acid substitution at amino acid residue 210 of SEQ ID NO:1 is a substitution to alanine; (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid; (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine; (4) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine; (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and b) growing the cell in the presence of a neomycin phosphotransferase substrate. In some embodiments, the method further comprises selecting for the host cell that grows in the presence of the neomycin phosphotransferase substrate.

In some embodiments of the method of using a plasmid or transposon, the wild-type NPT comprises an amino acid sequence that is at least 80%, at least 90%, or least 98% identical to SEQ ID NO:1. In certain embodiments of the method of using a plasmid or transposon, the wild-type NPT comprises an amino acid sequence that is at least 60%, at least 65%, least 70%, or at least 75% identical to SEQ ID NO:1.

In certain embodiments of the method of using a plasmid or transposon, the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine. In some embodiments of the method of using a plasmid or transposon, the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid. In certain embodiments of the method of using a plasmid or transposon, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine. In some embodiments of the method of using a plasmid or transposon, the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine. In certain embodiments of the method of using a plasmid or transposon, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine. In some embodiments of the method of using a plasmid or transposon, the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In certain embodiments of the method of using a plasmid or transposon, the non-naturally occurring NPT comprises the amino acid sequence of SEQ ID NO:38 (V36M, G210A). In some embodiments of the method of using a plasmid or transposon, the non-naturally occurring NPT comprises the amino acid sequence of SEQ ID NO:39 (V36M, E182D). In certain embodiments of the method of using a plasmid or transposon, the non-naturally occurring NPT comprises the amino acid sequence of SEQ ID NO:40 (V36M, Y218F). In some embodiments of the method of using a plasmid or transposon, the non-naturally occurring NPT comprises the amino acid sequence of SEQ ID NO:41 (D216G, D261N). In certain embodiments of the method of using a plasmid or transposon, the non-naturally occurring NPT comprises the amino acid sequence of SEQ ID NO:42 (V36M, Y218S). In some embodiments of the method of using a plasmid or transposon, the non-naturally occurring NPT comprises the amino acid sequence of SEQ ID NO:43 (V36M, D216G).

In certain embodiments of the method of using a plasmid or transposon, the host cell is a bacterial, yeast, mammalian or plant cell. In some embodiments, the host cell is a human cell.

In certain embodiments of the method of using a plasmid or transposon, wherein the plasmid or transposon further comprises a second nucleotide sequence encoding a protein or a non-coding RNA. In some embodiments, the protein is a viral protein. In certain embodiments, the protein is a therapeutic protein.

In certain embodiments of the method of using a plasmid or transposon, the neomycin phosphotransferase substrate is neomycin, kanamycin or G418.

In another aspect, provided herein are methods of making host cells comprising a) introducing into a population of host cells a first nucleic acid sequence comprising (i) a first nucleotide sequence encoding the non-naturally occurring NPT described herein, and (ii) a second nucleotide sequence comprising a transgene encoding a second protein or a non-coding RNA; b) growing the population of host cells in the presence of a neomycin phosphotransferase substrate to produce colonies; and c) selecting a colony of cells that grows in the presence of the neomycin phosphotransferase substrate. In some embodiments, the methods further comprise culturing the selected colony of cells.

In one embodiment, provided herein is a method of making host cells comprising a second nucleotide sequence comprising: a) introducing a population of host cells with a first nucleic acid sequence comprising (i) a first nucleotide sequence encoding the non-naturally occurring NPT, and (ii) a second nucleotide sequence comprising a transgene encoding a second protein or a non-coding RNA, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; b) growing the population of host cells in the presence of a neomycin phosphotransferase substrate to produce colonies; and c) selecting a colony of cells that grows in the presence of the neomycin phosphotransferase substrate.

In another aspect, provided herein is a method of making host cells comprising a second nucleotide sequence comprising: a) co-introducing into a population of host cells (i) a first nucleic acid sequence comprising a first nucleotide sequence encoding the non-naturally occurring NPT described herein, and (ii) a second nucleic acid sequence comprising a transgene encoding a second protein or a non-coding RNA; b) growing the population of host cells in the presence of a neomycin phosphotransferase substrate to produce colonies; and c) selecting a colony of cells that grows in the presence of the neomycin phosphotransferase substrate. In some embodiments, the methods further comprise culturing the selected colony of cells.

In some embodiments, provided herein is a method of making host cells comprising a second nucleotide sequence comprising: a) co-introducing into a population of host cells (i) a first nucleic acid sequence comprising a first nucleotide sequence encoding the non-naturally occurring NPT described herein, and (ii) a second nucleic acid sequence comprising a transgene encoding a second protein or a non-coding RNA, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; b) growing the population of host cells in the presence of a neomycin phosphotransferase substrate to produce colonies; and c) selecting a colony of cells that grows in the presence of the neomycin phosphotransferase substrate.

In some embodiments, provided herein is a method of making host cells comprising a second nucleotide sequence comprising: a) co-introducing into a population of host cells (i) a first nucleic acid sequence comprising a first nucleotide sequence encoding the non-naturally occurring NPT, and (ii) a second nucleic acid sequence comprising a transgene encoding a second protein or a non-coding RNA, wherein the non-naturally occurring NPT comprises the amino acid sequence of SEQ ID NO:1 with: (1) amino acid substitutions at positions 36 and 210 of SEQ ID NO:1, wherein the amino acid substitution at position 36 of SEQ ID NO:1 is a substitution to methionine and the amino acid substitution at position 210 of SEQ ID NO:1 is a substitution to alanine; (2) amino acid substitutions at positions 36 and 182 of SEQ ID NO:1, wherein the amino acid substitution at position 36 of SEQ ID NO:1 is a substitution to methionine and the amino acid substitution at position 182 of SEQ ID NO:1 is a substitution to aspartic acid; (3) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to phenylalanine; (4) amino acid substitutions at positions 216 and 261 of SEQ ID NO:1, wherein the amino acid substitution position 216 of SEQ ID NO:1 is a substitution to glycine and the amino acid substitution at position 261 of SEQ ID NO:1 is a substitution to asparagine; (5) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to serine; or (6) amino acid substitutions at positions 36 and 216 of SEQ ID NO:1, wherein the amino acid substitution at position 36 of SEQ ID NO:1 is a substitution to methionine and the amino acid substitution at position 216 of SEQ ID NO:1 is a substitution to glycine; b) growing the population of host cells in the presence of a neomycin phosphotransferase substrate to produce colonies; and c) selecting a colony of cells that grows in the presence of the neomycin phosphotransferase substrate.

In another aspect, provided herein are methods of making host cells comprising a second nucleotide sequence comprising: a) growing a population of hosts cells in the presence of a neomycin phosphotransferase substrate to produce colonies, wherein the population of host cells comprises a first nucleic acid sequence comprising (i) a first nucleotide sequence encoding a non-naturally occurring NPT described herein, and (ii) a second nucleotide sequence comprising a transgene encoding a second protein or a non-coding RNA; and b) selecting a colony of cells that grows in the presence of the neomycin phosphotransferase substrate. In some embodiments, the methods further comprise culturing the colony of selected cells.

In one embodiment, provided herein is a method of making host cells comprising a second nucleotide sequence comprising: a) growing a population of hosts cells in the presence of a substrate for neomycin phosphotransferase to produce colonies, wherein the population of host cells comprises a first nucleic acid sequence comprising (i) a first nucleotide sequence encoding the non-naturally occurring NPT, and (ii) a second nucleotide sequence comprising a transgene encoding a second protein or a non-coding RNA, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and b) selecting a colony of cells that grows in the presence of the neomycin phosphotransferase substrate. In some embodiments, the method further comprises culturing the selected colony of cells.

In some embodiments, a method of making host cells comprising a second nucleotide sequence comprises: a) growing a population of hosts cells in the presence of a substrate for neomycin phosphotransferase to produce colonies, wherein the population of host cells comprises (i) a first nucleic acid sequence comprising a first nucleotide sequence encoding the non-naturally occurring NPT, and (ii) a second nucleic acid sequence comprising a transgene encoding a second protein or a non-coding RNA; wherein the non-naturally occurring NPT comprises the amino acid sequence of SEQ ID NO:1 with: (1) amino acid substitutions at positions 36 and 210 of SEQ ID NO:1, wherein the amino acid substitution at position 36 of SEQ ID NO:1 is a substitution to methionine and the amino acid substitution at position 210 of SEQ ID NO:1 is a substitution to alanine; (2) amino acid substitutions at positions 36 and 182 of SEQ ID NO:1, wherein the amino acid substitution at position 36 of SEQ ID NO:1 is a substitution to methionine and the amino acid substitution at position 182 of SEQ ID NO:1 is a substitution to aspartic acid; (3) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to phenylalanine; (4) amino acid substitutions at positions 216 and 261 of SEQ ID NO:1, wherein the amino acid substitution position 216 of SEQ ID NO:1 is a substitution to glycine and the amino acid substitution at position 261 of SEQ ID NO:1 is a substitution to asparagine; (5) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to serine; or (6) amino acid substitutions at positions 36 and 216 of SEQ ID NO:1, wherein the amino acid substitution at position 36 of SEQ ID NO:1 is a substitution to methionine and the amino acid substitution at position 216 of SEQ ID NO:1 is a substitution to glycine; and b) selecting a colony of cells that grows in the presence of the neomycin phosphotransferase substrate. In some embodiments, the method further comprises culturing the selected colony of cells.

In another aspect, provided herein are host cells comprising a second nucleotide sequence produced by a method described herein.

In another aspect, provided herein is a method for manufacturing a stable cell line expressing a therapeutic protein or enzyme comprising: a) introducing one or more nucleic acid sequences into a population of host cells, wherein the one or more nucleic acid sequences comprise: (i) a first nucleic acid sequence encoding a non-naturally occurring neomycin phosphotransferase (NPT) described herein with neomycin phosphotransferase activity; b) selecting a cell from the population of cells of step (a) that grows in the presence of G418; and c) culturing the selected cell to produce a stable cell line expressing the therapeutic protein or enzyme.

In one embodiment, provided herein is a method for manufacturing a stable cell line expressing a therapeutic protein or enzyme comprising: a) introducing one or more nucleic acid sequences into a population of host cells, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and (ii) a second nucleic acid sequence encoding the therapeutic protein or enzyme; b) selecting a cell from the population of cells of step (a) that grows in the presence of G418; and c) culturing the selected cell to produce a stable cell line expressing the therapeutic protein or enzyme.

In some embodiments, provided herein is a method for manufacturing a stable cell line expressing a therapeutic protein or enzyme comprising: a) introducing one or more nucleic acid sequences into a population of host cells, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises the amino acid sequence of SEQ ID NO:1 with: (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine; (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid; (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine; (4) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine; (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and (ii) a second nucleic acid sequence encoding the therapeutic protein or enzyme; b) selecting a cell from the population of cells of step (a) that grows in the presence of G418; and c) culturing the selected cell to produce a stable cell line expressing the therapeutic protein or enzyme.

In some embodiments of the methods provided herein, the stable cell line is a mammalian cell line. In some embodiments of the methods provided herein, the stable cell line is a human cell line. In some embodiments, the stable cell line is a CHO, PER.C6, murine NS0, HEK293, fibrosarcoma HT-1080, murine Sp2/0, BHK, or murine C127 cell line. In some embodiments of the methods provided herein, the stable cell line expresses the therapeutic protein. In some embodiments of the methods provided herein, the therapeutic protein is an antibody or antibody fragment. In some embodiments, the stable cell line expresses the enzyme.

In another aspect, provided herein is a stable cell line produced by a method described herein. In some embodiments, stability of a cell line can be determined by measuring copy number of a transgene by quantitative methods, such as, e.g., qPCR or hybridization.

In some embodiments of the methods provided herein, the non-naturally occurring NPT has attenuated neomycin phosphotransferase activity as a selectable marker as compared to wild-type NPT.

In some embodiments of the methods provided herein, the wild-type NPT comprises an amino acid sequence that is at least 80%, at least 90%, or at least 98% identical to SEQ ID NO:1. In some embodiments of the methods provided, the wild-type NPT comprises an amino acid sequence that is at least 60%, at least 65%, at least 70% or at least 75% identical to SEQ ID NO:1.

In some embodiments of the methods provided herein, the non-naturally occurring NPT has attenuated neomycin phosphotransferase activity as a selectable marker as compared to wild-type NPT comprising the amino acid sequence of SEQ ID NO:1.

In some embodiments of the methods provided herein, bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT. In some embodiments, the bacterial cells are E. coli.

In some embodiments of the methods provided herein, mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells.

In some embodiments of the methods provided herein, G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT. In some embodiments, G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells.

In some embodiments of the methods provided herein, bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT, wherein the wild-type NPT comprises the amino acid sequence of SEQ ID NO:1.

In some embodiments of the methods provided herein, mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT, wherein the wild-type NPT comprises the amino acid sequence of SEQ ID NO: 1 In some embodiments, G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT, wherein the wild-type NPT comprises the amino acid sequence of SEQ ID NO:1. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells.

In some embodiments of the methods provided herein, the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine. In some embodiments, the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid. In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine. In some embodiments, the non-naturally occurring NPT comprises amino acid substitutions at amino acid residues corresponding to amino acid residues 216 and 261 of SEQ ID NO:1, wherein at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine. In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine. In some embodiments, the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In some embodiments of the methods provided herein, the non-naturally occurring NPT comprises the amino acid sequence of SEQ ID NO:38 (V36M, G210A).

In some embodiments of the methods provided herein, the non-naturally occurring NPT comprises the amino acid sequence of SEQ ID NO:39 (V36M, E182D).

In some embodiments of the methods provided herein, the non-naturally occurring NPT comprises the amino acid sequence of SEQ ID NO:40 (V36M, Y218F).

In some embodiments of the methods provided herein, the non-naturally occurring NPT comprises the amino acid sequence of SEQ ID NO:41 (D216G, D261N).

In some embodiments of the methods provided herein, the non-naturally occurring NPT comprises the amino acid sequence of SEQ ID NO:42 (V36M, Y218S).

In some embodiments of the methods provided herein, the non-naturally occurring NPT comprises the amino acid sequence of SEQ ID NO:43 (V36M, D216G).

In some embodiments of the methods provided herein, wherein a population of host cells are transfected or transformed, the host cells the population of host cells produces fewer colonies than a second population of host cells transfected or transformed with a second nucleic acid sequence and grown in the presence of the neomycin phosphotransferase substrate, wherein second nucleic acid sequence comprises a third nucleotide sequence encoding wild-type NPT protein and the second nucleotide sequence.

Host cells can, for example, be mammalian cells. In some embodiments, the mammalian cells are HEK293 cells, CHO cells, PER.C6 cells, murine NS0 cells, fibrosarcoma HT-1080 cells, murine Sp2/0 cells, BHK cells, or murine C127 cells. In some embodiments, the cells are human cells.

In some embodiments of the methods provided herein, the neomycin phosphotransferase substrate is neomycin, kanamycin, or G418.

In some embodiments of the methods provided herein, the protein is a therapeutic protein or an antigen.

In some embodiments of the methods provided herein, the non-coding RNA is shRNA, miRNA, antisense RNA, guide RNA for Crispr nucleases, catalytic RNA, ribosomal RNA or tRNA.

In another aspect, provided herein is a method of making a virus producer cell line comprising: a) introducing one or more nucleic acid sequences into a population of host cells, wherein the one or more nucleic acid sequences comprise: (i) a first nucleic acid sequence encoding a non-naturally occurring neomycin phosphotransferase (NPT) described herein with neomycin phosphotransferase activity; b) selecting a cell from the population of cells that grows in the presence in of a neomycin phosphotransferase substrate; and c) propagating the selected cell to produce a virus producer cell line. The virus producer cell line may be used to produce virus for, e.g., gene therapy or cancer therapy.

In one embodiment, provided herein is a method of making a virus producer cell line comprising: a) introducing one or more nucleic acid sequences into a population of host cells, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and (ii) a second nucleic acid sequence encoding one or more viral proteins; b) selecting a cell from the population of cells that grows in the presence in of a neomycin phosphotransferase substrate; and c) propagating the selected cell to produce a virus producer cell line. In some embodiments, the one or more viral proteins includes a capsid protein, an envelope protein, a viral protein necessary for replication, or a combination thereof.

In some embodiments, a method of making a virus producer cell line comprises: a) introducing one or more nucleic acid sequences into a population of host cells, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises the amino acid sequence of SEQ ID NO:1 with: (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine; (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid; (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine; (4) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine; (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and (ii) a second nucleic acid sequence encoding one or more viral proteins; b) selecting a cell from the population of cells that grows in the presence in of a neomycin phosphotransferase substrate; and c) propagating the selected cell to produce a virus producer cell line. In some embodiments, the one or more viral proteins includes a capsid protein, an envelope protein, a viral protein necessary for replication, or a combination thereof.

In some embodiments of the methods provided herein, the non-naturally occurring NPT has attenuated neomycin phosphotransferase activity as a selectable marker as compared to wild-type NPT.

In some embodiments of the methods provided herein, the wild-type NPT comprises an amino acid sequence that is at least 80%, at least 90%, or at least 98% identical to SEQ ID NO:1. In some embodiments of the methods provided, the wild-type NPT comprises an amino acid sequence that is at least 60%, at least 65%, at least 70% or at least 75% identical to SEQ ID NO:1.

In some embodiments of the methods provided herein, the non-naturally occurring NPT has attenuated neomycin phosphotransferase activity as a selectable marker as compared to wild-type NPT comprising the amino acid sequence of SEQ ID NO:1.

In some embodiments of the methods provided herein, the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine. In some embodiments, the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid. In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine. In some embodiments, the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine. In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine. In some embodiments, the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In some embodiments of the methods provided herein, the non-naturally occurring NPT comprises the amino acid sequence of SEQ ID NO:38 (V36M, G210A).

In some embodiments of the methods provided herein, the non-naturally occurring NPT comprises the amino acid sequence of SEQ ID NO:39 (V36M, E182D).

In some embodiments of the methods provided herein, the non-naturally occurring NPT comprises the amino acid sequence of SEQ ID NO:40 (V36M, Y218F).

In some embodiments of the methods provided herein, the non-naturally occurring NPT comprises the amino acid sequence of SEQ ID NO:41 (D216G, D261N).

In some embodiments of the methods provided herein, the non-naturally occurring NPT comprises the amino acid sequence of SEQ ID NO:42 (V36M, Y218S).

In some embodiments of the methods provided herein, the non-naturally occurring NPT comprises the amino acid sequence of SEQ ID NO:43 (V36M, D216G).

In some embodiments of the methods provided herein, the cell line is a mammalian cell line. In some embodiments of the methods provided herein, the cell line is a human cell line. In some embodiments of the methods provided herein, the cell line is a CHO, PER.C6, murine NS0, HEK293, fibrosarcoma HT-1080, murine Sp2/0, BHK, or murine C127 cell line.

In some embodiments of the methods provided herein, the one or more viral proteins includes an AAV capsid protein.

In some embodiments of the methods provided herein, the one or more viral proteins includes an AAV capsid protein and AAV rep protein.

In some embodiments of the methods provided herein, the one or more viral proteins includes an envelope protein.

In some embodiments of the methods provided herein, the one or more viral proteins includes adenovirus E1 region proteins required for adenovirus replication.

In some embodiments of the methods provided herein, the one or more viral proteins includes a retroviral envelope protein.

In some embodiments of the methods provided herein, the one or more viral proteins includes a retroviral gag protein.

In some embodiments of the methods provided herein, the one or more viral proteins includes a retroviral reverse transcriptase.

In some embodiments of the methods provided herein, the one or more viral proteins includes a retroviral envelope protein, gag protein and reverse transcriptase.

In another aspect, provided herein is a virus producer cell line comprising one or more nucleic acid sequences, wherein the one or more nucleic acid sequences comprise: (i) a first nucleic acid sequence encoding a non-naturally occurring neomycin phosphotransferase (NPT) described herein with neomycin phosphotransferase activity, and (ii) second nucleic acid sequence encoding one or more viral proteins. In some embodiments, the one or more viral proteins includes a capsid protein, an envelope protein, a viral protein necessary for replication, or a combination thereof.

In one embodiment, provided herein is a virus producer cell line comprising one or more nucleic acid sequences, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and (ii) a second nucleic acid sequence encoding one or more viral proteins. In some embodiments, the one or more viral proteins includes a capsid protein, an envelope protein, a viral protein necessary for replication, or a combination thereof.

In some embodiments, provided herein is a virus producer cell line comprising one or more nucleic acid sequences, wherein the one or more nucleic acid sequences comprises: (i) a first nucleic acid sequence encoding a non-naturally occurring neomycin phosphotransferase (NPT) with neomycin phosphotransferase activity, wherein the non-naturally occurring NPT comprises the amino acid sequence of SEQ ID NO:1 with: (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine; (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid; (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine; (4) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine; (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and (ii) a second nucleic acid sequence encoding one or more viral proteins. In some embodiments, the one or more viral proteins includes a capsid protein, an envelope protein, a viral protein necessary for replication, or a combination thereof.

In some embodiments of the virus producer cell line, the non-naturally occurring NPT has attenuated neomycin phosphotransferase activity as a selectable marker as compared to wild-type NPT.

In some embodiments of the virus producer cell line, the wild-type NPT comprises an amino acid sequence that is at least 80%, at least 90%, or at least 98% identical to SEQ ID NO:1. In some embodiments of the virus producer cell line, the wild-type NPT comprises an amino acid sequence that is at least 60%, at least 65%, at least 70%, or at least 75% identical to SEQ ID NO:1. In some embodiments of the virus producer cell line, the non-naturally occurring NPT has attenuated neomycin phosphotransferase activity as a selectable marker as compared to wild-type NPT comprising the amino acid sequence of SEQ ID NO:1.

In some embodiments of the virus producer cell line, the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine. In some embodiments of the virus producer cell line, the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid. In some embodiments of the virus producer cell line, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine. In some embodiments of the virus producer cell line, the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine. In some embodiments of the virus producer cell line, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine. In some embodiments of the virus producer cell line, the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In some embodiments of the virus producer cell line, the non-naturally occurring NPT comprises the amino acid sequence of SEQ ID NO:38 (V36M, G210A). In some embodiments of the virus producer cell line, the non-naturally occurring NPT comprises the amino acid sequence of SEQ ID NO:39 (V36M, E182D). In some embodiments of the virus producer cell line, the non-naturally occurring NPT comprises the amino acid sequence of SEQ ID NO:40 (V36M, Y218F). In some embodiments of the virus producer cell line, the non-naturally occurring NPT comprises the amino acid sequence of SEQ ID NO:41 (D216G, D261N). In some embodiments of the virus producer cell line, the non-naturally occurring NPT comprises the amino acid sequence of SEQ ID NO:42 (V36M, Y218S). In some embodiments of the virus producer cell line, the non-naturally occurring NPT comprises the amino acid sequence of SEQ ID NO:43 (V36M, D216G).

In some embodiments of the virus producer cell line, the cell line is a mammalian cell line. In some embodiments of the virus producer cell line, the cell line is a human cell line. In some embodiments of the virus producer cell line, the cell line is a CHO, PER.C6, murine NS0, HEK293, fibrosarcoma HT-1080, murine Sp2/0, BHK, or murine C127 cell line.

In some embodiments of the virus producer cell line, the one or more viral proteins includes an AAV capsid protein. In some embodiments of the virus producer cell line, the one or more viral proteins includes an AAV capsid protein and AAV rep protein. In some embodiments of the virus producer cell line, the one or more viral proteins includes an envelope protein. In some embodiments of the virus producer cell line, the one or more viral proteins includes adenovirus E1 region proteins required for adenovirus replication. In some embodiments of the virus producer cell line, the one or more viral proteins includes a retroviral envelope protein. In some embodiments of the virus producer cell line, the one or more viral proteins includes a retroviral gag protein. In some embodiments of the virus producer cell line, the one or more viral proteins includes a retroviral reverse transcriptase. In some embodiments of the virus producer cell line, the one or more viral proteins includes a retroviral envelope protein, gag protein and reverse transcriptase.

In one aspect, provided herein is a method for manufacturing a mammalian cell line expressing an antigen comprising: a) introducing one or more nucleic acid sequences into a population of host cells, wherein the one or more nucleic acid sequences comprise: (i) a first nucleic acid sequence encoding a non-naturally occurring neomycin phosphotransferase (NPT) described herein with neomycin phosphotransferase activity, and (ii) a second nucleic acid sequence encoding an antigen; b) selecting a cell from the population of cells of step (a) that grows in the presence of G418; and c) culturing the selected cell to produce a cell line expressing the antigen. In some embodiments, the antigen is used to immunize a mammalian subject (e.g., a human) or induce an immune response in a mammalian subject (e.g., human). The antigen may also be used in vitro or ex vivo.

In one embodiment, provided herein is a method for manufacturing a mammalian cell line expressing an antigen comprising: a) introducing one or more nucleic acid sequences into a population of host cells, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring neomycin NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and (ii) a second nucleic acid sequence encoding an antigen; b) selecting a cell from the population of cells of step (a) that grows in the presence of G418; and c) culturing the selected cell to produce a cell line expressing the antigen.

In some embodiments, provided herein is a method for manufacturing a mammalian cell line expressing an antigen comprising: a) introducing one or more nucleic acid sequences into a population of host cells, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring neomycin NPT comprises the amino acid sequence of SEQ ID NO:1 with: (1) amino acid substitutions at amino acid residues 36 and 210 of SEQ ID NO: 1, wherein the amino acid substitution at amino acid residue 36 of SEQ ID NO:1 is a substitution to methionine and the amino acid substitution at amino acid residue 210 of SEQ ID NO:1 is a substitution to alanine; (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid; (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine; (4) amino acid substitutions at amino acid residues 216 and 261 of SEQ ID NO:1, wherein the amino acid substitution at the amino acid residue 216 of SEQ ID NO:1 is a substitution to glycine and the amino acid substitution at amino acid residue 261 of SEQ ID NO:1 is a substitution to asparagine; (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and (ii) a second nucleic acid sequence encoding an antigen; b) selecting a cell from the population of cells of step (a) that grows in the presence of G418; and c) culturing the selected cell to produce a cell line expressing the antigen.

In some embodiments of the methods provided herein, the non-naturally occurring NPT has attenuated neomycin phosphotransferase activity as a selectivity marker as compared to wild-type NPT.

In some embodiments of the methods provided herein, the wild-type NPT comprises an amino acid sequence that is at least 80%, at least 90%, or at least 98% identical to SEQ ID NO:1. In some embodiments of the methods provided herein, the wild-type NPT comprises an amino acid sequence that is at least 60%, at least 65%, at least 70% or at least 75% identical to SEQ ID NO:1.

In some embodiments of the methods provided herein, the non-naturally occurring NPT has attenuated neomycin phosphotransferase activity as a selectable marker as compared to wild-type NPT comprising the amino acid sequence of SEQ ID NO:1.

In some embodiments of the methods provided herein, the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine. In some embodiments, the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid. In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine. In some embodiments, the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine. In some embodiments, the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine. In some embodiments, the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 216 of SEQ ID NO:1 is a substitution to glycine.

In some embodiments of the methods provided herein, the non-naturally occurring NPT comprises the amino acid sequence of SEQ ID NO:38 (V36M, G210A).

In some embodiments of the methods provided herein, the non-naturally occurring NPT comprises the amino acid sequence of SEQ ID NO:39 (V36M, E182D).

In some embodiments of the methods provided herein, the non-naturally occurring NPT comprises the amino acid sequence of SEQ ID NO:40 (V36M, Y218F).

In some embodiments of the methods provided herein, the non-naturally occurring NPT comprises the amino acid sequence of SEQ ID NO:41 (D216G, D261N).

In some embodiments of the methods provided herein, the non-naturally occurring NPT comprises the amino acid sequence of SEQ ID NO:42 (V36M, Y218S).

In some embodiments of the methods provided herein, the non-naturally occurring NPT comprises the amino acid sequence of SEQ ID NO:43 (V36M, D216G).

In some embodiments of a method for manufacturing a mammalian cell line, the cell line is a mammalian cell line. In some embodiments of a method for manufacturing a mammalian cell line, the cell line is a human cell line. In some embodiments of a method for manufacturing a mammalian cell line, the cell line is a CHO, PER.C6, murine NS0, HEK293, fibrosarcoma HT-1080, murine Sp2/0, BHK, or murine C127 cell line.

In some embodiments of a method for manufacturing a mammalian cell line, the antigen is a viral antigen, a bacterial antigen, or a fungal antigen. In some embodiments of a method for manufacturing a mammalian cell line, the antigen is a cancer antigen.

In another aspect, provided herein are antigen producing cell lines comprising one or more nucleic acid sequences, wherein the one or more nucleic acid sequences comprise: (i) a first nucleic acid sequence encoding a non-naturally occurring neomycin phosphotransferase (NPT) described herein with neomycin phosphotransferase activity; and (ii) a second nucleic acid sequence encoding one or more antigens.

In another aspect, provided herein is an antigen producing cell line comprising one or more nucleic acid sequences, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and (ii) a second nucleic acid sequence encoding one or more antigens.

In some embodiments, provided herein is an antigen producing cell line comprising one or more nucleic acid sequences, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises the amino acid sequence of SEQ ID NO: 1 with: (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine; (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid; (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine; (4) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine; (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and (ii) a second nucleic acid sequence encoding one or more antigens.

In some embodiments of an antigen producing cell line provided herein, the non-naturally occurring NPT has attenuated neomycin phosphotransferase activity as a selectable marker as compared to wild-type NPT.

In some embodiments of an antigen producing cell line provided herein, the wild-type NPT comprises an amino acid sequence that is at least 80%, at least 90%, or at least 98% identical to SEQ ID NO:1.

In some embodiments of an antigen producing cell line provided herein the wild-type NPT comprises an amino acid sequence that is at least 60%, at least 65%, at least 70%, or at least 65% identical to SEQ ID NO:1.

In some embodiments of an antigen producing cell line provided herein, the non-naturally occurring NPT has attenuated neomycin phosphotransferase activity as a selectable marker as compared to wild-type NPT comprising the amino acid sequence of SEQ ID NO:1.

In some embodiments, the NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine. In some embodiments, the NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid. In some embodiments, the NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine. In some embodiments, the NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine. In some embodiments, the NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine. In some embodiments, the NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

In some embodiments of an antigen producing cell line provided herein, the non-naturally occurring NPT comprises the amino acid sequence of SEQ ID NO:38 (V36M, G210A). In some embodiments, the non-naturally occurring NPT comprises the amino acid sequence of SEQ ID NO:39 (V36M, E182D). In some embodiments of an antigen producing cell line provided herein, the non-naturally occurring NPT comprises the amino acid sequence of SEQ ID NO:40 (V36M, Y218F). In some embodiments of an antigen producing cell line provided herein, the non-naturally occurring NPT comprises the amino acid sequence of SEQ ID NO:41 (D216G, D261N). In some embodiments of an antigen producing cell line provided herein, the non-naturally occurring NPT comprises the amino acid sequence of SEQ ID NO:42 (V36M, Y218S). In some embodiments of an antigen producing cell line provided herein, the non-naturally occurring NPT comprises the amino acid sequence of SEQ ID NO:43 (V36M, D216G).

In some embodiments of an antigen producing cell line provided herein, the cell line is a mammalian cell line. In some embodiments of an antigen producing cell line provided herein, the cell line is a human cell line. In some embodiments of an antigen producing cell line provided herein, the cell line is a CHO, PER.C6, murine NS0, HEK293, fibrosarcoma HT-1080, murine Sp2/0, BHK, or murine C127 cell line.

In some embodiments of an antigen producing cell line provided herein, the one or more antigens is a viral antigen, a bacterial antigen, or a fungal antigen. In some embodiments of an antigen producing cell line provided herein, the one or more antigens is a cancer antigen.

In another aspect, provided herein is a selectable marker means for conferring resistance to kanamycin when introduced into a bacterial cell, and to G418 when introduced into a mammalian cell. In some embodiments, the selectable marker means comprises a nucleic acid sequence of SEQ ID NO:20. In some embodiments, the selectable marker means comprises a nucleic acid sequence of SEQ ID NO:32. In some embodiments, the selectable marker means comprises a nucleic acid sequence of SEQ ID NO:33. In some embodiments, the selectable marker means comprises a nucleic acid sequence of SEQ ID NO:34. In some embodiments, the selectable marker means comprises a nucleic acid sequence of SEQ ID NO:36. In some embodiments, the selectable marker means comprises a nucleic acid sequence of SEQ ID NO:37.

In another aspect, provided herein is a method for manufacturing a producer cell line comprising: a) transforming a bacterial or mammalian cell with an expression vector comprising nucleic acid sequence encoding one or more viral proteins and a means for growing in the presence of kanamycin if the transformed cell is a bacterial cell and for growing in the presence of G418 if the transformed cell is a mammalian cell to make a transformed cell; and b) culturing the transformed cell in the presence of kanamycin or G418 to obtain a producer cell line, wherein the producer cell line expresses one or more viral proteins from AAV, adenovirus, retrovirus, lentivirus, herpes simplex virus, vaccinia virus or baculovirus.

In another aspect, provided herein is a method for selecting a cell with stable chromosomal integration of an exogenous nucleic acid sequence comprising: a) transforming a population of eukaryotic cells with an exogenous nucleic acid sequence comprising a means for growing in the presence of G418; b) culturing the population of transformed cells in the presence of G418 to produce colonies of transformed cells capable of growing in the presence of G418; and c) selecting a cell from a colony produced in step (b) to obtain a cell with a stable chromosomal integration of the exogenous nucleic acid.

In some embodiments, the exogenous nucleic acid sequence further comprises a transgene, and the selected cell expresses the transgene.

In some embodiments, the exogenous nucleic acid sequence disrupts expression of a gene endogenous to the selected cell.

In another aspect, provided herein is a method for selecting a mammalian cell with a stable episome comprising: a) transforming a population of mammalian cells with a plasmid comprising a means for growing in the presence of G418; b) culturing the population of transformed cells in the presence of G418 to produce colonies of transformed cells capable of growing in the presence of G418; and c) selecting a cell from a colony produced in step (b) to obtain a cell with a stable episome comprising the plasmid.

In some embodiments, the plasmid further comprises an EBNA1 OriP nucleic acid sequence and the selected cell expresses EBNA1.

In one aspect, provided herein is a method for selecting a mammalian cell transiently expressing a transgene comprising: a) introducing into a population of mammalian cells a nucleic acid encoding a transgene and a means for growing in the presence of G418; b) culturing the population of mammalian cells in the presence of G418 for 48-72 hours; and c) selecting a mammalian cell from the cultured population of mammalian cells that grows in the presence of G418, wherein the selected mammalian cell transiently expresses the transgene.

In some embodiments, the transgene comprises nucleic acid sequences encoding a Crispr endonuclease or a Crispr guide RNA.

In some embodiments, the means is nucleotide sequence encoding a non-naturally occurring neomycin phosphotransferase comprising the amino acid sequence selected from the group of SEQ ID NO: 38, 39, 40, 41, 42 or 43.

### 6. BRIEF DESCRIPTION OF THE DRAWINGS

The foregoing summary, as well as the following detailed description of specific embodiments of the present application, will be better understood when read in conjunction with the appended drawings. It should be understood, however, that the application is not limited to the precise embodiments shown in the drawings.

FIG. 1 illustrates a representative expression vector (plasmid P313) as described herein.

FIG. 2 depicts a construct including transposon elements (“Leapin left” and “Leapin Right”), Human Elongation Factor alpha promoter (“EF Ia”), mCherry coding region with polyadenylation signal (“pA”), NPT coding region (“Kan/NEO”), and an origin of replication (“pMB1 Ori”).

FIG. 3 depicts results from a colony formation assay described herein.

FIG. 4 demonstrates mCherry expression in stable pools of HEK293 cells transformed with constructs expressing mCherry and NPT proteins (labeled “NEO”) as compared to untransformed cells (leftmost tube) with no color.

FIG. 5 shows a graph of transgene (mCherry) copy number in HEK293 cells transformed with constructs P724 encoding wild-type NPT, P725 encoding NPT mutant #1 (V36M; G210A), or P726 encoding NPT mutant #2 (V36M; E182D) and where the constructs either include (+) or do not include (−) transposase elements.

FIGS. 6A-B shows an alignment of aminoglycoside phosphotransferases adapted from Shaw et al., Microbiological Reviews 57: 138-163 (1993). SEQ ID NOS: 18, 19 and 45-62 have been assigned to the sequences depicted in FIGS. 6A-6B.

### 7. DETAILED DESCRIPTION

The present disclosure is based, in part, on the surprising discovery of NPTs with particular amino acid substitutions having phosphotransferase activities that are significantly reduced as compared to wild-type NPT. The use of nucleic acid sequences encoding NPTs as described herein provide a substantial advantage as a selectable maker for the selection and creation of transformed cell lines, which in addition to a gene of interest, express a mutated NPT, which gives the transformed cells a selective advantage over non-transformed cells.

As used herein and in the appended claims, the singular forms “a,” “an,” and “the” include plural reference unless the context clearly dictates otherwise.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, WI), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word length (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

The terms “wild-type NPT” and wild-type neomycin phosphotransferase” are used interchangeably herein and are understood by the skilled person. Generally, a wild-type NPT refers to a neomycin phosphotransferase, which prevails among organisms in nature. In some embodiments, a wild-type NPT is an aminoglycoside phosphotransferase3′-II. In certain embodiments, a wild-type NPT is an aminoglycoside phosphotransferase3′-IIa. In some embodiments, a wild-type NPT is neomycin phosphotransferase from Tn5 (aminoglycoside phosphotransferase3′-IIa). In a specific embodiment, a wild-type NPT comprises the amino acid sequence of SEQ ID NO:1. In another specific embodiment, a wild-type NPT comprises the amino acid sequence of SEQ ID NO:44. In other embodiments, a wild-type NPT comprises with an amino acid sequence other than SEQ ID NO:1 or SEQ ID NO:44.

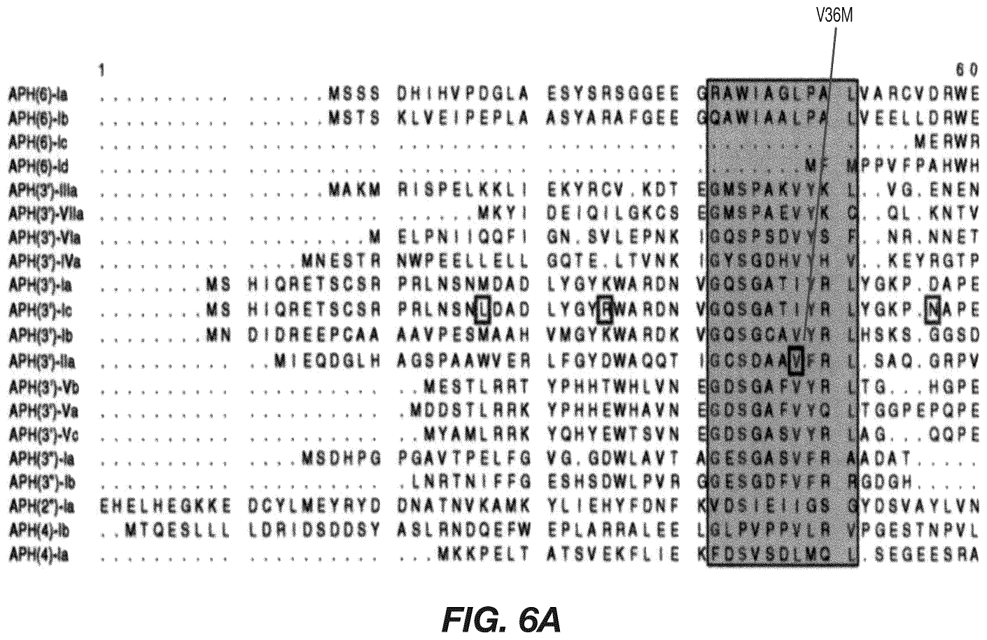

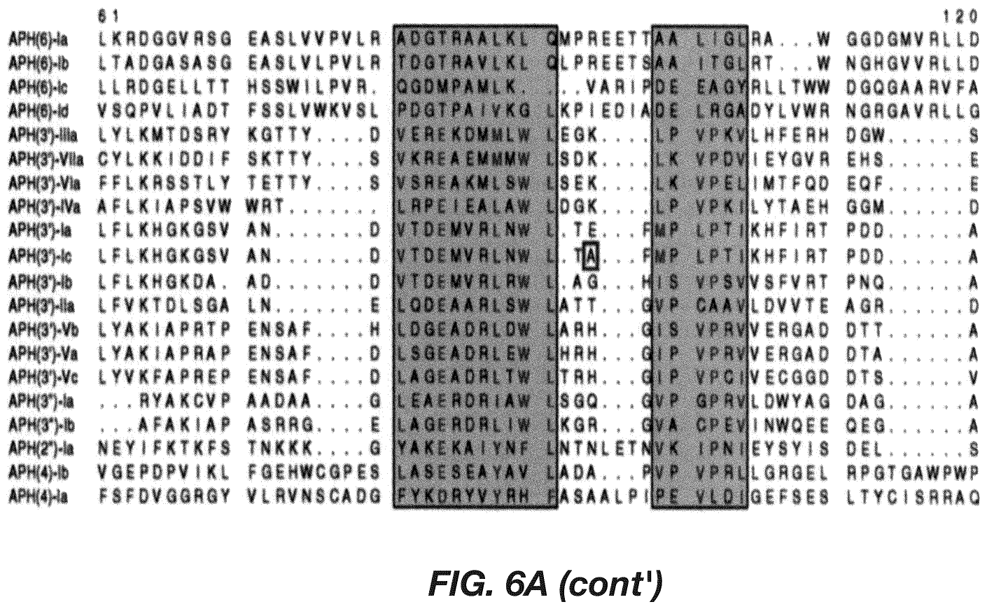

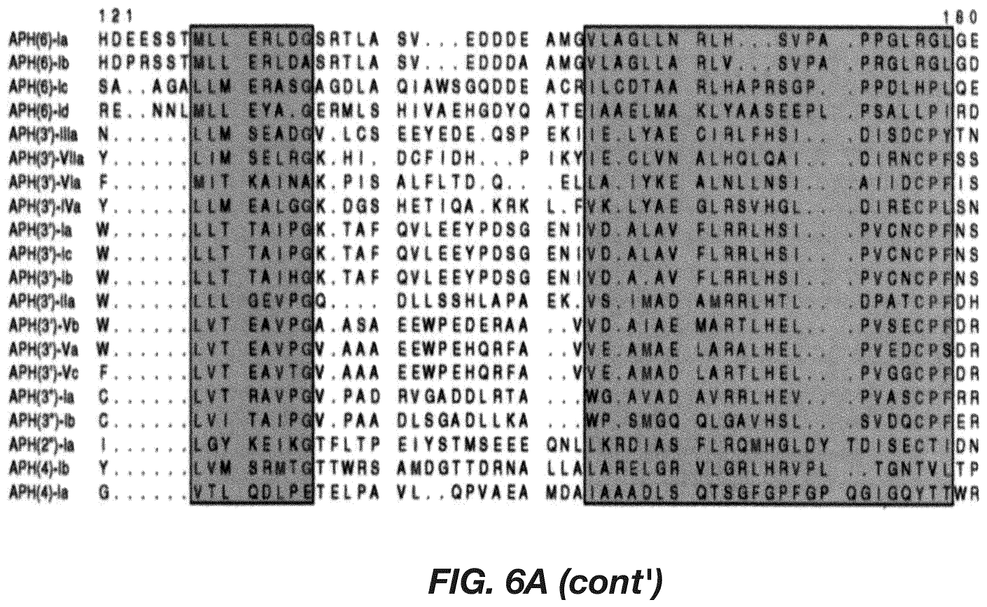

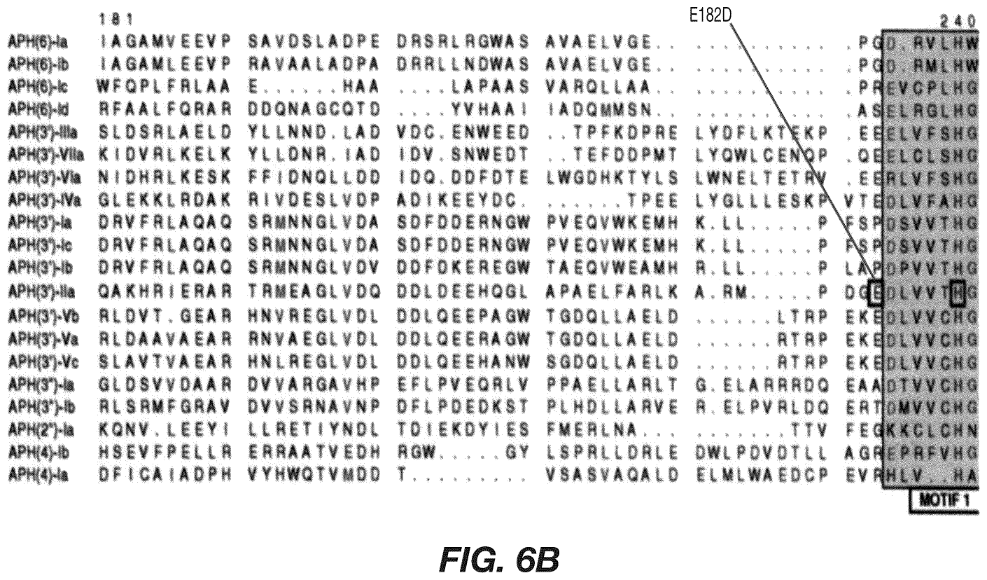

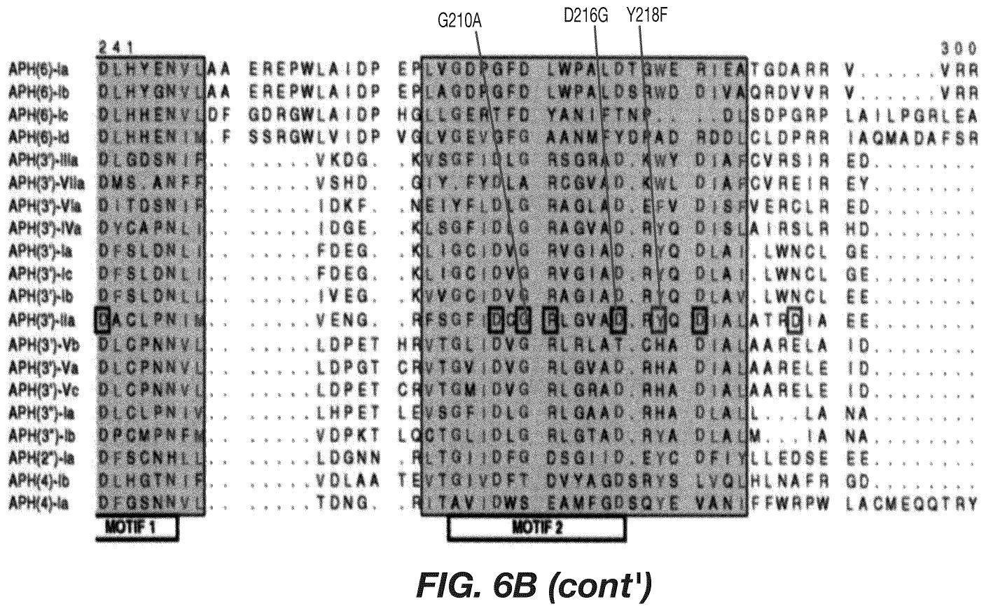

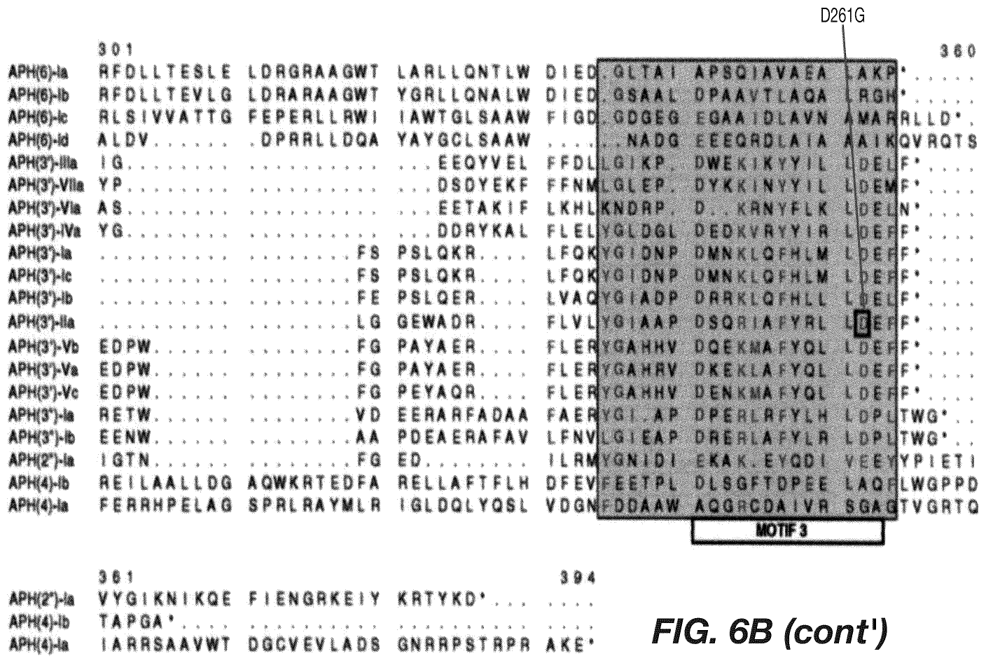

Descriptions of amino acid positions of substitutions in a NPT described herein are relative to the amino acid position of SEQ ID NO: 1. For example, amino acid substitutions of a wild-type NPT at amino acid residues corresponding to amino acid residues 36 and 210 of SEQ ID NO:1 refers to a wild-type NPT with amino acid substitutions at amino acid residues of the wild-type NPT that correspond to amino acid residues 36 and 210 of SEQ ID NO:1 in an alignment, such as provided in FIGS. 6A-6B. In FIGS. 6A-6B the sequence of APH(3′)-IIa is the reference sequence (i.e., the amino acid sequence that corresponds to SEQ ID NO:1) and to which other wild-type NPT compared. An exemplary nucleic acid sequence encoding the amino acid sequence of SEQ ID NO:1 is provided as SEQ ID NO:6.

As used herein, the phrase “selectable marker means” refers to a NPT mutant or a non-naturally occurring NPT described herein, or a nucleotide sequence encoding a NPT mutant or a non-naturally occurring NPT described herein, which allows for the growth of host cells in the presence of a neomycin phosphotransferase substrate (e.g., neomycin, kanamycin or G418, or a derivative thereof).

As used herein, the phrase “means for growing in the presence” of a neomycin phosphotransferase substrate (e.g., neomycin, kanamycin or G418, or a derivative thereof) refers to a NPT mutant or a non-naturally occurring NPT described herein, or a nucleotide sequence encoding a NPT mutant or a non-naturally occurring NPT described herein, which allows for the growth of host cells in the presence of the neomycin phosphotransferase substrate.

### 7.1 Neomycin Phosphotransferase (NPT) Proteins

In one aspect, provided herein are NPT mutants that differ in amino acid sequence from wild-type NPT and that have altered phosphotransferase activity (e.g., reduced phosphotransferase activity) as compared to wild-type NPT. In one embodiment, the NPT mutants comprise one, two, or more amino acid substitutions described herein in wild-type NPT (e.g., in Table 1 or Table 2), or a combination thereof. In specific embodiments, NPT mutants provided herein are non-naturally occurring NPT proteins. In certain embodiments, NPT mutants provided herein are isolated NPT proteins. In a specific embodiment, the NPT mutants provided herein have attenuated activity as a selectable marker as compared to wild-type NPT. In a particular embodiment, a NPT mutant has reduced enzymatic activity compared to the corresponding wild-type NPT in an assay described herein or known to one of skill in the art. For example, the enzymatic activity of a NPT may be measured in an in vitro kinase assay, such as described in Kocabiyik and Perlin, Biochem Biophys Res Commun 185(3): 925-931 (1992). The enzymatic activity of the NPT mutant is compared to the corresponding wild-type NPT under the same conditions. Alternatively or in addition, the enzymatic activity NPT may be measured indirectly by assessing colony formation by bacteria (e.g., E. coli) transformed with a plasmid(s) encoding the NPT mutant after a certain period of time (e.g., 36 hours, 48 hours, 72 hours, or more) on plates containing a certain amount of kanamycin (e.g., 25 μg/ml, 75 μg/ml, or 100 μg/ml) and appropriate nutrients for growth of the bacteria as well as appropriate conditions (e.g., temperature, etc.) for the bacteria to grow. The colony formation of bacteria transformed with a nucleotide sequence encoding the NPT mutant is compared to the colony formation of the same species of bacteria transformed with a nucleotide sequence encoding the corresponding wild-type NPT grown under the same growth conditions as the bacteria transformed a nucleotide sequence encoding with the NPT mutant, wherein fewer and/or smaller colonies formed by the bacteria transformed with a nucleotide sequence encoding the NPT mutant relative colonies formed by bacteria transformed with a plasmid(s) encoding the wild-type NPT indicates that the enzymatic activity and/or protein stability of the NPT mutant is attenuated. Another example of an indirect assay to assess the enzymatic activity the NPT mutant involves comparing the colony formation by mammalian cells transfected or transformed with DNAs encoding the NPT mutant protein to the colony formation by mammalian cells transfected with DNAs encoding the corresponding wild-type NPT, wherein both populations of mammalian cells are grown on plates or another appropriate type of container containing media necessary for growth and a certain concentration of G418 (e.g., 500 μg/ml) under the same conditions (e.g., the same temperature, CO₂, etc.) for a certain period of time (e.g., 2 weeks, 2.5 weeks, 3 weeks, or more), wherein a reduction in colony formation by the mammalian cells transfected with the NPT mutant as compared to colony formation by the mammalian cells transfected with the wild-type NPT indicates that the NPT mutant has attenuated enzymatic activity.

Another example of an indirect assay to assess the enzymatic activity of the NPT gene involves measuring the proportion of the cells transfected with a mammalian expression construct that stably integrate the construct into host chromosomes and form colonies when diluted and plated in tissue culture dishes in media containing the selective agent. For example, HEK293 cells transfected with plasmids designed to express wild-type or mutant NPT isoforms are plated at 2E6 cells or less into 150 mm tissue culture dished in DMEM medium containing 10% Fetal Bovine serum and G418 at 600 μg/ml and cultured at 37° C. at 8% CO₂ for 2 weeks. Media is removed and cells are stained with 10 mls 0.4% Methylene Blue in 50% methanol by incubating at room temperature 10 min. The stain is removed, cells are washed with 100% methanol, air dried and photographed. A decrease in the proportion of colonies: number of cells plated using a mutant NPT expression construct relative to wild-type NPT expression construct indicates that the mutant has attenuated enzymatic activity.

In certain embodiments, a NPT mutant with reduced activity as compared to wild-type NPT exhibits 0.001% to 10% of the phosphotransferase activity of wild-type NPT (e.g., SEQ ID NO:1 or SEQ ID NO:44) as determined in a suitable assay. In some embodiments, a NPT mutant with reduced activity as compared to wild-type NPT exhibits 0.001% to 8% of the phosphotransferase activity of wild-type NPT (e.g., SEQ ID NO:1 or SEQ ID NO:44) as determined in a suitable assay. In certain embodiments, a NPT mutant with reduced activity as compared to wild-type NPT exhibits 0.01% to 6% of the phosphotransferase activity of wild-type NPT (e.g., SEQ ID NO:1 or SEQ ID NO:44) as determined in a suitable assay. NPT phosphotransferase activity can be measured using any of the assays known in art (see, e.g., Kocabiyik and Perlin, Biochem Biophys Res Commun 185(3): 925-931 (1992) and references cited therein for an exemplary method of assaying phosphotransferase activity) or described herein (e.g., colony formation). In certain embodiments, a NPT mutant has one or two amino acid substitutions in an amino acid sequence of a wild-type NPT, wherein the amino acid substitutions at the amino acid residues of the wild-type NPT correspond to one or two of the amino acid residues of SEQ ID NO:1 recited in Table 1 or Table 2. In some embodiments, a NPT mutant has one amino acid substitution in an amino acid sequence of a wild-type NPT, wherein the amino acid substitution is at the amino acid residue of the wild-type NPT that corresponds to one of the amino acid residues of SEQ ID NO:1 recited in Table 1 or Table 2. In specific embodiments, the NPT mutant has reduced activity as assessed by a technique known to one of skill in the art or described herein. In certain embodiments, a NPT mutant has two amino acid substitutions in an amino acid sequence of a wild-type NPT, wherein the amino acid substitutions are at two of the amino acid residues of the wild-type NPT that correspond to two of the amino acid residues of SEQ ID NO:1 recited in Table 1 or Table 2. In specific embodiments, the NPT mutant has reduced activity as assessed by a technique known to one of skill in the art or described herein.

In certain embodiments, a NPT mutant has one or two amino acid substitutions in an amino acid sequence of a neomycin phosphotransferase variant with wild-type neomycin phosphotransferase activity, wherein the amino acid substitutions at the amino acid residues of the variant correspond to one or two of the amino acid residues of SEQ ID NO:1 recited in Table 1 or Table 2. In some embodiments, a NPT mutant has one amino acid substitution in an amino acid sequence of a neomycin phosphotransferase variant with wild-type neomycin phosphotransferase activity, wherein the amino acid substitution is at the amino acid residue of the variant that corresponds to one of the amino acid residues of SEQ ID NO:1 recited in Table 1 or Table 2. In specific embodiments, the NPT mutant has reduced activity as assessed by a technique known to one of skill in the art or described herein. In certain embodiments, a NPT mutant has two amino acid substitution in an amino acid sequence of a neomycin phosphotransferase variant with wild-type neomycin phosphotransferase activity, wherein the amino acid substitutions are at two of the amino acid residues of the variant that correspond to two of the amino acid residues of SEQ ID NO:1 recited in Table 1 or Table 2. In specific embodiments, the NPT mutant has reduced activity as assessed by a technique known to one of skill in the art or described herein.

In certain embodiments, a NPT mutant provided herein differs from a wild-type NPT by having a methionine at a position corresponding to the amino acid at position 36 of SEQ ID NO:1, and an alanine at a position corresponding to the amino acid at position 210 of SEQ ID NO:1. In certain embodiments, the NPT mutant differs from a wild-type NPT by having a methionine at a position corresponding to the amino acid at position 36 of SEQ ID NO:1, and an aspartic acid at a position corresponding to the amino acid at position 182 of SEQ ID NO:1. In certain embodiments, the NPT mutant differs from a wild-type NPT by having a methionine at a position corresponding to the amino acid at position 36 of SEQ ID NO:1, and an phenylalanine at a position corresponding to the amino acid at position 218 of SEQ ID NO:1. In certain embodiments, the NPT mutant differs from a wild-type NPT by having a glycine at a position corresponding to the amino acid at position 216 of SEQ ID NO:1, and an asparagine at a position corresponding to the amino acid at position 261 of SEQ ID NO:1. In certain embodiments, the NPT mutant differs from a wild-type NPT by having a methionine at a position corresponding to the amino acid at position 36 of SEQ ID NO:1, and an serine at a position corresponding to the amino acid at position 218 of SEQ ID NO:1. In certain embodiments, the NPT mutant differs from a wild-type NPT by having a methionine at a position corresponding to the amino acid at position 36 of SEQ ID NO:1, and a glycine at a position corresponding to the amino acid at position 216 of SEQ ID NO:1. In specific embodiments, the NPT mutant has reduced activity as assessed by a technique known to one of skill in the art or described herein.

In certain embodiments, provided herein is a non-naturally occurring NPT with neomycin phosphotransferase activity, wherein the non-naturally occurring NPT comprises an amino acid sequence of a wild-type neomycin phosphotransferase with amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 210 of SEQ ID NO:1 is a substitution to alanine. In some embodiments, provided herein is a non-naturally occurring NPT with neomycin phosphotransferase activity, wherein the non-naturally occurring NPT comprises an amino acid sequence of a wild-type neomycin phosphotransferase with amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 182 of SEQ ID NO:1 is a substitution to aspartic acid. In certain embodiments, provided herein is a non-naturally occurring NPT with neomycin phosphotransferase activity, wherein the non-naturally occurring NPT comprises an amino acid sequence of a wild-type neomycin phosphotransferase with amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to phenylalanine. In some embodiments, provided herein is a non-naturally occurring NPT with neomycin phosphotransferase activity, wherein the non-naturally occurring NPT comprises an amino acid sequence of a wild-type neomycin phosphotransferase with amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at amino acid residue corresponding to amino acid residue 261 of SEQ ID NO:1 is a substitution to asparagine. In certain embodiments, provided herein is a non-naturally occurring NPT with neomycin phosphotransferase activity, wherein the non-naturally occurring NPT comprises an amino acid sequence of a wild-type neomycin phosphotransferase with amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to serine. In some embodiments, provided herein are non-naturally occurring NPT with neomycin phosphotransferase activity, wherein the non-naturally occurring NPT comprises an amino acid sequence of a wild-type neomycin phosphotransferase with amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 216 of SEQ ID NO:1 is a substitution to glycine. In specific embodiments, the non-naturally occurring NPT has reduced activity as assessed by a technique known to one of skill in the art or described herein.

In some embodiments, a wild-type NPT comprises an amino acid sequence that is at least 50%, at least 55%, or at least 60% identical to SEQ ID NO:1 or SEQ ID NO:44. In certain embodiments, a wild-type NPT comprises an amino acid sequence that is at least 65%, at least 70%, or at least 75% identical to SEQ ID NO:1 or SEQ ID NO:44. In some embodiments, a wild-type NPT comprises an amino acid sequence that is at least 80%, at least 90%, or at least 98% identical to SEQ ID NO:1 or SEQ ID NO:44. In certain embodiments, a wild-type NPT comprises an amino acid sequence that is 50% to 75%, 50% to 80%, 50% to 60%, 75% to 95%, or 85% to 95% identical to SEQ ID NO:1 or SEQ ID NO:44. In some embodiments, Motif 1, Motif 2, or Motif 3 of a wild-type sequence is identical to Motif 1, Motif 2 or Motif 3, respectively, of SEQ ID NO:1 or SEQ ID NO:44. In certain embodiments, Motif 1, Motif 2, and Motif 3 of a wild-type sequence are identical to Motif 1, Motif 2 and Motif 3, respectively, of SEQ ID NO:1 or SEQ ID NO:44. In some embodiments, a combination of Motif 1, Motif 2, and Motif 3 of a wild-type sequence are identical to a combination of Motif 1, Motif 2 and Motif 3, respectively, of SEQ ID NO:1 or SEQ ID NO:44. In some embodiments, Motif 1, Motif 2, or Motif 3 of a wild-type sequence is at least 85%, at least 90%, or at least 95% identical to Motif 1, Motif 2 or Motif 3, respectively, of SEQ ID NO:1 or SEQ ID NO:44. In certain embodiments, Motif 1, Motif 2, and Motif 3 of a wild-type sequence are at least 85%, at least 90%, or at least 95% identical to Motif 1, Motif 2 and Motif 3, respectively, of SEQ ID NO:1 or SEQ ID NO:44. In some embodiments, a combination of Motif 1, Motif 2, and Motif 3 of a wild-type sequence are at least 85%, at least 90%, or at least 95% identical to a combination of Motif 1, Motif 2 and Motif 3, respectively, of SEQ ID NO:1 or SEQ ID NO:44. In some embodiments, a combination of Motif 1, Motif 2, and Motif 3 of a wild-type sequence are at least 98% or at least 99% identical to a combination of Motif 1, Motif 2 and Motif 3, respectively, of SEQ ID NO:1 or SEQ ID NO:44.

In certain embodiments, provided herein are non-naturally occurring NPT with neomycin phosphotransferase activity, wherein the non-naturally occurring NPT comprises an amino acid sequence of a neomycin phosphotransferase variant with wild-type neomycin phosphotransferase activity with amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 210 of SEQ ID NO:1 is a substitution to alanine. In some embodiments, provided herein are non-naturally occurring NPT with neomycin phosphotransferase activity, wherein the non-naturally occurring NPT comprises an amino acid sequence of a neomycin phosphotransferase variant with wild-type neomycin phosphotransferase activity with amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 182 of SEQ ID NO:1 is a substitution to aspartic acid. In certain embodiments, provided herein is a non-naturally occurring NPT with neomycin phosphotransferase activity, wherein the non-naturally occurring NPT an amino acid sequence of a neomycin phosphotransferase variant with wild-type neomycin phosphotransferase activity with amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to phenylalanine. In some embodiments, provided herein is a non-naturally occurring NPT with neomycin phosphotransferase activity, wherein the non-naturally occurring NPT comprises an amino acid sequence of a neomycin phosphotransferase variant with wild-type neomycin phosphotransferase activity with amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at amino acid residue corresponding to amino acid residue 261 of SEQ ID NO:1 is a substitution to asparagine. In certain embodiments, provided herein is a non-naturally occurring NPT with neomycin phosphotransferase activity, wherein the non-naturally occurring NPT comprises an amino acid sequence of a neomycin phosphotransferase variant with wild-type neomycin phosphotransferase activity with amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to serine. In some embodiments, provided herein are non-naturally occurring NPT with neomycin phosphotransferase activity, wherein the non-naturally occurring NPT comprises an amino acid sequence of a neomycin phosphotransferase variant with wild-type neomycin phosphotransferase activity with amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 216 of SEQ ID NO:1 is a substitution to glycine. In specific embodiments, the non-naturally occurring NPT mutant has reduced activity as assessed by a technique known to one of skill in the art or described herein.

In certain embodiments, a neomycin phosphotransferase variant with wild-type neomycin phosphotransferase activity comprises an amino acid sequence that is at least 60%, at least 65%, at least 70%, or at least 75% identical to SEQ ID NO:1 or SEQ ID NO:44. In certain embodiments, a neomycin phosphotransferase variant with wild-type neomycin phosphotransferase activity comprises an amino acid sequence that is at least 80%, at least 90%, or at least 98% identical to SEQ ID NO:1 or SEQ ID NO:44. In some embodiments, Motif 1, Motif 2, or Motif 3 of a neomycin phosphotransferase variant with wild-type neomycin phosphotransferase activity is identical to Motif 1, Motif 2 or Motif 3, respectively, of SEQ ID NO:1 or SEQ ID NO:44. In certain embodiments, Motif 1, Motif 2, and Motif 3 of a neomycin phosphotransferase variant with wild-type neomycin phosphotransferase activity are identical to Motif 1, Motif 2 and Motif 3, respectively, of SEQ ID NO:1 or SEQ ID NO:44. See, e.g., FIGS. 6A-6B for the location of Motifs 1, 2, and 3 of aminoglycoside transferases. In some embodiments, a combination of Motif 1, Motif 2, and Motif 3 of a neomycin phosphotransferase variant with wild-type neomycin phosphotransferase activity are identical to a combination of Motif 1, Motif 2 and Motif 3, respectively, of SEQ ID NO:1 or SEQ ID NO:44. In some embodiments, Motif 1, Motif 2, or Motif 3 of a neomycin phosphotransferase variant with wild-type neomycin phosphotransferase activity is at least 85%, at least 90%, or at least 95% identical to Motif 1, Motif 2 or Motif 3, respectively, of SEQ ID NO:1 or SEQ ID NO:44. In certain embodiments, Motif 1, Motif 2, and Motif 3 of a neomycin phosphotransferase variant with wild-type neomycin phosphotransferase activity are at least 85%, at least 90%, or at least 95% identical to Motif 1, Motif 2 and Motif 3, respectively, of SEQ ID NO:1 or SEQ ID NO:44. In some embodiments, a combination of Motif 1, Motif 2, and Motif 3 of a neomycin phosphotransferase variant with wild-type neomycin phosphotransferase activity are at least 85%, at least 90%, or at least 95% identical to a combination of Motif 1, Motif 2 and Motif 3, respectively, of SEQ ID NO:1 or SEQ ID NO:44. In some embodiments, a combination of Motif 1, Motif 2, and Motif 3 of a neomycin phosphotransferase variant with wild-type neomycin phosphotransferase activity are at least 98% or at least 99% identical to a combination of Motif 1, Motif 2 and Motif 3, respectively, of SEQ ID NO:1 or SEQ ID NO:44.

In certain embodiments, a NPT mutant comprises the amino acid sequence of SEQ ID NO:1 with one or two amino acid substitutions. In specific embodiment, a NPT mutant is any one of the NPT mutants listed Table 1 provided herein. In another specific embodiment, a NPT mutant is any one of the NPT mutants listed in Table 2 provided herein.

In certain embodiments, a NPT mutant provided herein differs from a SEQ ID NO:1 or SEQ ID NO:44 by having a methionine at amino acid position 36 of SEQ ID NO:1 or SEQ ID NO:44, and an alanine at amino acid position 210 of SEQ ID NO:1 or SEQ ID NO:44. In certain embodiments, a NPT mutant differs from SEQ ID NO:1 or SEQ ID NO:44 by having a methionine at amino acid position 36 of SEQ ID NO:1 or SEQ ID NO:44, and an aspartic acid at amino acid at position 182 of SEQ ID NO:1 or SEQ ID NO:44. In certain embodiments, a NPT mutant differs from SEQ ID NO:1 or SEQ ID NO:44 by having a methionine at amino acid position 36 of SEQ ID NO:1 or SEQ ID NO:44, and a phenylalanine at amino acid position 218 of SEQ ID NO:1 or SEQ ID NO:44. In certain embodiments, a NPT mutant differs from SEQ ID NO:1 or SEQ ID NO:44 by having a glycine at amino acid position 216 of SEQ ID NO:1 or SEQ ID NO:44, and an asparagine amino acid position 261 of SEQ ID NO:1 or SEQ ID NO:44. In certain embodiments, a NPT mutant differs from SEQ ID NO:1 or SEQ ID NO:44 by having a methionine at amino acid position 36 of SEQ ID NO:1 or SEQ ID NO:44, and an serine at amino acid position 218 of SEQ ID NO:1 or SEQ ID NO:44. In certain embodiments, a NPT mutant differs from SEQ ID NO:1 or SEQ ID NO:44 by having a methionine amino acid position 36 of SEQ ID NO:1 or SEQ ID NO:44, and a glycine at amino acid position 216 of SEQ ID NO:1 or SEQ ID NO:44. In certain embodiments, a NPT mutant provided herein is a double point NPT mutant of SEQ ID NO:1. For example, in some embodiments, a NPT mutant comprises the amino acid sequence of SEQ ID NO:38. In certain embodiments, a NPT mutant comprises the amino acid sequence of SEQ ID NO:39. In some embodiments, a NPT mutant comprises the amino acid sequence of SEQ ID NO40. In certain embodiments, a NPT mutant is comprises the amino acid sequence of SEQ ID NO41. In some embodiments, a NPT mutant comprises the amino acid sequence of SEQ ID NO42. In other embodiments, a NPT mutant comprises the amino acid sequence of SEQ ID NO43.

In certain embodiments, a NPT mutant provided herein comprises an amino acid sequence encoded by the nucleotide sequence of SEQ ID NO:12. In some embodiments, a NPT mutant provided herein comprises an amino acid sequence encoded by the nucleotide sequence of SEQ ID NO:13. In certain embodiments, a NPT mutant provided herein comprises an amino acid sequence encoded by the nucleotide sequence of SEQ ID NO:14. In some embodiments, a NPT mutant provided herein comprises an amino acid sequence encoded by the nucleotide sequence of SEQ ID NO:15. In certain embodiments, a NPT mutant provided herein comprises an amino acid sequence encoded by the nucleotide sequence of SEQ ID NO:16. In some embodiments, a NPT mutant provided herein comprises an amino acid sequence encoded by the nucleotide sequence of SEQ ID NO:17. In certain embodiments, a NPT mutant provided herein comprises an amino acid sequence encoded by the nucleotide sequence of SEQ ID NO:18. In some embodiments, a NPT mutant provided herein comprises an amino acid sequence encoded by the nucleotide sequence of SEQ ID NO:19. In certain embodiments, a NPT mutant provided herein comprises an amino acid sequence encoded by the nucleotide sequence of SEQ ID NO:21. In some embodiments, a NPT mutant provided herein comprises an amino acid sequence encoded by the nucleotide sequence of SEQ ID NO:22. In certain embodiments, a NPT mutant provided herein comprises an amino acid sequence encoded by the nucleotide sequence of SEQ ID NO:23. In some embodiments, a NPT mutant provided herein comprises an amino acid sequence encoded by the nucleotide sequence of SEQ ID NO:24. In certain embodiments, a NPT mutant provided herein comprises an amino acid sequence encoded by the nucleotide sequence of SEQ ID NO:25. In some embodiments, a NPT mutant provided herein comprises an amino acid sequence encoded by the nucleotide sequence of SEQ ID NO:26. In certain embodiments, a NPT mutant provided herein comprises an amino acid sequence encoded by the nucleotide sequence of SEQ ID NO:27. In some embodiments, a NPT mutant provided herein comprises an amino acid sequence encoded by the nucleotide sequence of SEQ ID NO:28. In certain embodiments, a NPT mutant provided herein comprises an amino acid sequence encoded by the nucleotide sequence of SEQ ID NO:29. In some embodiments, a NPT mutant provided herein comprises an amino acid sequence encoded by the nucleotide sequence of SEQ ID NO:30. In certain embodiments, a NPT mutant provided herein comprises an amino acid sequence encoded by the nucleotide sequence of SEQ ID NO:31. In certain embodiments, a NPT mutant provided herein comprises an amino acid sequence encoded by the nucleotide sequence of SEQ ID NO:35.

In certain embodiments, a NPT mutant provided herein comprises an amino acid sequence encoded by the nucleotide sequence of SEQ ID NO:20. In some embodiments, a NPT mutant provided herein comprises an amino acid sequence encoded by the nucleotide sequence of SEQ ID NO:32. In certain embodiments, a NPT mutant provided herein comprises an amino acid sequence encoded by the nucleotide sequence of SEQ ID NO:33. In some embodiments, a NPT mutant provided herein comprises an amino acid sequence encoded by the nucleotide sequence of SEQ ID NO:34. In certain embodiments, a NPT mutant provided herein comprises an amino acid sequence encoded by the nucleotide sequence of SEQ ID NO:36. In some embodiments, a NPT mutant provided herein comprises an amino acid sequence encoded by the nucleotide sequence of SEQ ID NO:37.

In certain embodiments, bacterial cells transfected or transformed with a nucleotide sequence encoding a NPT mutant as provided herein exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding the corresponding wild-type NPT (e.g., SEQ ID NO:1). “Reduced colony formation” can, for example, be a reduction of 0.001% to 75% of colonies relative to kanamycin resistant colonies of bacterial cells transfected or transformed with a nucleotide sequence encoding wild-type NPT. In some embodiments, the reduced colony formation is a reduction of 0.001% to 10% relative to kanamycin resistant colonies of bacterial cells transfected or transformed with a nucleotide sequence encoding wild-type NPT. In certain embodiments, the reduced colony formation is a reduction of 0.01% to 6% relative to kanamycin resistant colonies of bacterial cells transfected or transformed with a nucleotide sequence encoding wild-type NPT.

In some embodiments, mammalian cells transfected or transformed with a nucleotide sequence encoding a NPT mutant as provided herein exhibit reduced colony formation as assessed by a colony formation assay after 2 weeks growth on tissue-culture plates containing 500 μg/mL geneticin (G418) relative to mammalian cells transfected or transformed with a nucleotide sequence encoding wild-type NPT (e.g., SEQ ID NO:1). “Reduced colony formation” can, for example, be a reduction of 0.001% to 75% of colonies relative to G418 resistant colonies of mammalian cells transfected with a nucleotide sequence encoding wild-type NPT. In some embodiments, the reduced colony formation is a reduction of 0.001% to 10% relative to G418 resistant colonies of mammalian cells transfected with wild-type NPT. In certain embodiments, the reduced colony formation is a reduction of 0.01% to 6% relative to G418 resistant colonies of mammalian cells transfected with a nucleotide sequence encoding wild-type NPT.

A NPT mutant or non-naturally occurring NPT described herein confers resistance to certain antibiotics (e.g., neomycin, kanamycin, G418, or derivatives of any of the foregoing). In a specific embodiment, the expression of a NPT mutant or non-naturally occurring NPT described herein by a cell enables the cell to grow in the presence of a neomycin phosphotransferase substrate (e.g., neomycin, kanamycin, G418, or derivatives of any of the foregoing). In some embodiments, a mutant NPT or a non-naturally occurring NPT comprises an amino acid sequence described in Section 8, infra.

### 7.2 Nucleic Acid Sequences

In one aspect, provided herein are nucleic acids encoding a NPT mutant described herein. In a specific embodiment, provided herein are nucleic acid sequences comprising a nucleotide sequence encoding a NPT mutant described herein. In another specific embodiment, provided herein are nucleic acid sequences comprising a nucleotide sequence encoding a non-naturally occurring NPT described herein. Due to the degeneracy of the code, any nucleotide sequence that encodes a NPT mutant or non-naturally occurring NPT is encompassed by the present disclosure. In certain embodiments, the nucleotide sequence encoding a NPT mutant or non-naturally occurring NPT is codon optimized (e.g., codon optimized for expression in a particular subject or a cell(s) from a particular subject). Techniques known in the art may be used to codon optimize a nucleotide sequence encoding a NPT mutant or non-naturally occurring NPT. The nucleic acid sequence or nucleotide sequence may further comprise one or more regulatory elements (e.g., a promoter, an enhancer, etc.). In some embodiments, nucleic acid sequence or nucleotide sequence may further comprises one, two or more, or all of the following: a promoter, an enhancer, an intron, and a poly-A sequence. In some embodiments, nucleic acid sequence or nucleotide sequence may further comprises a promoter and an origin of replication sequence.

In specific embodiments, a nucleic acid sequence or nucleotide sequence is isolated from the nucleic acid sequence in which it is found in nature. In certain embodiments, a nucleic acid sequence or nucleotide sequence is isolated from the organism in which it is found in nature. Moreover, an “isolated” nucleic acid sequence, such as a cDNA molecule, can be substantially free of other cellular material, or culture medium when produced by recombinant techniques, or substantially free of chemical precursors or other chemicals when chemically synthesized. For example, the language “substantially free” includes preparations of polynucleotide or nucleic acid molecule having less than about 15%, 10%, 5%, 2%, 1%, 0.5%, or 0.1%) (in particular less than about 10%) of other material, e.g., cellular material, culture medium, other nucleic acid molecules, chemical precursors and/or other chemicals.

As used herein, the terms “nucleic acid” and “nucleotide” include deoxyribonucleotides, deoxyribonucleic acids, ribonucleotides, and ribonucleic acids, and polymeric forms thereof, and includes either single- or double-stranded forms. In certain embodiments, the terms “nucleic acid” and “nucleotide” include known analogues of natural nucleotides, for example, peptide nucleic acids (“PNA”s), that have similar binding properties as the reference nucleic acid. In some embodiments, the terms “nucleic acid” and “nucleotide” refer to deoxyribonucleic acids (e.g., cDNA or DNA). In other embodiments, the terms “nucleic acid” and “nucleotide” refer to ribonucleic acids (e.g., mRNA or RNA).

In certain embodiments, provided herein is a nucleic acid sequence comprising the nucleotide sequence of SEQ ID NO:12. In some embodiments, provided herein is a nucleic acid sequence comprising the nucleotide sequence of SEQ ID NO:13. In certain embodiments, provided herein is a nucleic acid sequence comprising the nucleotide sequence of SEQ ID NO:14. In some embodiments, provided herein is a nucleic acid sequence comprising the nucleotide sequence of SEQ ID NO:15. In certain embodiments, provided herein is a nucleic acid sequence comprising the nucleotide sequence of SEQ ID NO:16. In some embodiments, provided herein is a nucleic acid sequence comprising the nucleotide sequence of SEQ ID NO:17. In certain embodiments, provided herein is a nucleic acid sequence comprising the nucleotide sequence of SEQ ID NO:18. In some embodiments, provided herein is a nucleic acid sequence comprising the nucleotide sequence of SEQ ID NO:19. In certain embodiments, provided herein is a nucleic acid sequence comprising the nucleotide sequence of SEQ ID NO:21. In some embodiments, provided herein is a nucleic acid sequence comprising the nucleotide sequence of SEQ ID NO:22. In certain embodiments, provided herein is a nucleic acid sequence comprising the nucleotide sequence of SEQ ID NO:23. In some embodiments, provided herein is a nucleic acid sequence comprising the nucleotide sequence of SEQ ID NO:24. In certain embodiments, provided herein is a nucleic acid sequence comprising the nucleotide sequence of SEQ ID NO:25. In some embodiments, provided herein is a nucleic acid sequence comprising the nucleotide sequence of SEQ ID NO:26. In certain embodiments, provided herein is a nucleic acid sequence comprising the nucleotide sequence of SEQ ID NO:27. In some embodiments, provided herein is a nucleic acid sequence comprising the nucleotide sequence of SEQ ID NO:28. In certain embodiments, provided herein is a nucleic acid sequence comprising the nucleotide sequence of SEQ ID NO:29. In some embodiments, provided herein is a nucleic acid sequence comprising the nucleotide sequence of SEQ ID NO:30. In certain embodiments, provided herein is a nucleic acid sequence comprising the nucleotide sequence of SEQ ID NO:31. In certain embodiments, provided herein is a nucleic acid sequence comprising the nucleotide sequence of SEQ ID NO:35.

In certain embodiments, provided herein is a nucleic acid sequence comprising the nucleotide sequence of SEQ ID NO:20. In some embodiments, provided herein is a nucleic acid sequence comprising the nucleotide sequence of SEQ ID NO:32. In certain embodiments, provided herein is a nucleic acid sequence comprising the nucleotide sequence of SEQ ID NO:33. In some embodiments, provided herein is a nucleic acid sequence comprising the nucleotide sequence of SEQ ID NO:34. In certain embodiments, provided herein is a nucleic acid sequence comprising the nucleotide sequence of SEQ ID NO:36. In some embodiments, provided herein is a nucleic acid sequence comprising the nucleotide sequence of SEQ ID NO:37.

In certain embodiments, provided herein is a nucleic acid sequence comprising a first nucleotide sequence encoding a NPT mutant or non-naturally occurring NPT described herein and a second nucleotide sequence. The second nucleotide sequence may encode a protein of interest or a non-coding RNA, or may comprise a nucleotide sequence that disrupts an endogenous gene in a host cell. In some embodiments, provided herein is a nucleic acid sequence, comprising a first nucleotide sequence encoding a NPT mutant or non-naturally occurring NPT described herein and a second nucleotide sequence encoding a protein of interest or a non-coding RNA. In certain embodiments, the nucleic acid sequence may further comprise additional nucleotide sequences (e.g., transposon elements). The nucleic acid sequence may further comprise one or more regulatory elements (e.g., a promoter, an enhancer, etc.), origin of replication, and/or poly-A sequence. In certain embodiments, the first and second nucleotide sequences are operably linked to the same promoter. In other embodiments, the first and second nucleotide sequences are operably linked to different promoters.

In certain embodiments, provided herein is a nucleic acid sequence comprising a first nucleotide sequence encoding a NPT mutant or non-naturally occurring NPT described herein, a second nucleotide sequence of a first fragment of a gene of interest, and a third nucleotide sequence of a second fragment of the gene of interest, wherein the second nucleotide sequence flanks the first nucleotide sequence at the 5′ end and the third nucleotide sequence flanks the first nucleotide sequence at the 3′ end, wherein the first and second fragments facilitate recombination and disruption of the gene of interest. In some embodiments, the nucleic acid sequence further comprises a loxP nucleotide sequence upstream of the second nucleotide sequence and a loxP nucleotide sequence downstream of the third nucleotide sequence. See, e.g., Guldener et al., Nucleic Acids Research 24 (13): 2519-2524 (1996) for how such a nucleic acid sequence may be produced and used. The nucleic acid sequence may further comprise one or more regulatory elements (e.g., a promoter, an enhancer, etc.), poly-A sequence, etc.

In certain embodiments, provided herein is a nucleic acid sequence comprising a first nucleotide sequence encoding a NPT mutant or non-naturally occurring NPT described herein, a second nucleotide sequence encoding a protein of interest, a third nucleotide sequence comprising a first transposase sequence, and a fourth nucleotide sequence comprising a second transposase sequence, wherein the third nucleotide sequence is upstream of the first and second nucleotide sequences, and wherein the fourth nucleotide sequence is downstream of the first and second nucleotide sequences. In some embodiments, the first transposase sequence is the Leap-In left transposase and the second transposase is the Leap-In transposase. The nucleic acid sequence may further comprise one or more regulatory elements (e.g., a promoter, an enhancer, etc.), origin of replication, and/or a poly-A sequence.

In a specific embodiment, a nucleic acid sequence is one described in Section 8, infra.

In specific embodiments, provided herein is a nucleic acid sequence comprising a first nucleotide sequence encoding a NPT mutant or non-naturally occurring NPT described herein and a transgene. The transgene may be a native gene sequence, or it may be modified, e.g., to include codon optimization for adapting for expression in a particular host cell. The transgene may comprise a nucleotide sequence encoding a protein of interest or a non-coding RNA. In specific embodiments, the transgene is operably linked to one or more regulatory elements (e.g., a promoter, enhancer, etc.).

A protein of interest can, for example, be a therapeutic protein or a detectable marker. In certain embodiments, a protein of interest is a hormone, growth factor, antibody, viral protein, enzyme, cytokine, or a fragment thereof. In certain embodiments, the fragment is at least 8, at least 9, at least 10, at least 11, or at least 12 amino acids in length. In some embodiments, a protein of interest is an antigen (e.g., a viral, bacterial, fungal, or cancer antigen). In certain embodiments, a protein of interest is a viral protein, such as a capsid protein, an envelope protein, or a protein required for viral replication. The viral protein may be an adeno-associated virus (AAV), adenovirus, retrovirus, lentivirus, herpes simplex virus, vaccinia virus, or baculovirus protein. In some embodiments, a protein of interest is a peptide or polypeptide, which may be useful as a therapeutic or in a diagnostic assay.

A non-coding RNA can, for example, be an antisense RNA, microRNA (miRNA), short hairpin RNA (shRNA), long non-coding RNA, catalytic RNA (including, for example, a ribozyme), ribosomal RNA, tRNA, or guide RNA for CRISPR nucleases.

In some embodiments of a nucleic acid sequence provided herein, the nucleic acid sequence further comprises a nucleotide sequence encoding a selectable maker other than a NPT protein. A selectable marker, when introduced into a cell, confers a trait suitable for artificial selection. A selectable marker can, for example, confer resistance to an antibiotic, or it can code for an enzyme necessary for growth of eukaryotic cells under certain culturing conditions. Selectable markers are well known in the art. In certain embodiments, the selectable marker is beta-lactamase that confers ampicillin resistance. In some embodiments, the selectable marker is a fluorescent protein. In some embodiments, the term “selectivity marker” is used interchangeably with “selectable marker.”

Selection markers that can be used, include but not limited to, the herpes simplex virus thymidine kinase (Wigler et al, Cell 11:223 (1977)), hypoxanthineguanine phosphoribosyltransferase (Szybalska & Szybalski, Proc. Natl. Acad. Sci. USA 48:202 (1992), and adenine phosphoribosyltransferase (Lowy et al, Cell 22:8-17 (1980)) genes can be employed in tk-, hgprt- or aprt-cells, respectively. Also, antimetabolite resistance can be used as the basis of selection for the following genes: dhfr, which confers resistance to methotrexate (Wigler et al, Proc. Natl. Acad. Sci. USA 77:357 (1980); O'Hare et al., Proc. Natl. Acad. Sci. USA 78: 1527 (1981)); gpt, which confers resistance to mycophenolic acid (Mulligan & Berg, Proc. Natl. Acad. Sci. USA 78:2072 (1981)); md hygro, which confers resistance to hygromycin (Santerre et al, Gene 30: 147 (1984)).

### 7.3 Vectors

In another aspect, provided herein is a vector comprising a nucleic acid sequence comprising a nucleotide sequence encoding a NPT mutant or a non-naturally occurring NPT described herein. In specific embodiments, provided herein is a vector comprises a nucleic acid sequence or nucleotide sequence described herein (e.g., in Section 7.2 or Section 8). In some embodiments, provided herein is a vector comprising a first nucleotide sequence encoding a NPT mutant or non-naturally occurring NPT described herein and a second nucleotide sequence encoding a protein of interest or a non-coding RNA. In certain embodiments, provided herein is a vector comprising a first nucleotide sequence encoding a NPT mutant or non-naturally occurring NPT described herein, a second nucleotide sequence of a first fragment of a gene of interest, and a third nucleotide sequence of a second fragment of the gene of interest, wherein the second nucleotide sequence flanks the first nucleotide sequence at the 5′ end and the third nucleotide sequence flanks the first nucleotide sequence at the 3′ end, and wherein the first and second fragments facilitate recombination and disruption of the gene of interest. In some embodiments, the vector further comprises a loxP nucleotide sequence upstream of the second nucleotide sequence and a loxP nucleotide sequence downstream of the third nucleotide sequence.

In a specific embodiment, a vector is one described in Section 8, infra.

Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible or repressible promoter. A number of expression vectors capable of delivering nucleic acids to a cell are known in the art and can be used herein for production of a protein or non-coding RNA in the cell. Conventional cloning techniques or artificial gene synthesis can be used to generate a recombinant expression vector according to embodiments provided herein. Such techniques are well known to those skilled in the art in view of the present disclosure.

In certain embodiments, the vector is a cloning vector comprising nucleic acid encoding a NPT mutant. Cloning vectors can, for example, be a plasmid, phage, virus, cosmid, episome, or bacterial artificial chromosome. See also Section 7.4 for vectors, including expression vectors, encompassed herein.

### 7.4 Methods for Expression of NPT Mutant

In one aspect, provided herein are methods for producing a NPT mutant or a non-naturally occurring NPT described herein and optionally, one or more additional proteins or non-coding RNAs.

In certain aspects, provided herein are cells (e.g., host cells) expressing (e.g., recombinantly expressing) a NPT mutant or a non-naturally occurring NPT described herein and optionally, one or more additional proteins or one or more non-coding RNAs, or both. In another aspect, provided herein are vectors (e.g., expression vectors) comprising a nucleic acid sequence, wherein the nucleic acid sequence comprises a nucleotide sequence encoding a NPT mutant or a non-naturally occurring NPT described herein and optionally, one or more nucleotide sequences encoding one or more additional proteins or non-coding RNAs, or both for recombinant expression in host cells (e.g., mammalian cells). Also provided herein are host cells comprising a nucleic acid sequence comprising a nucleotide encoding a NPT mutant or a non-naturally occurring NPT described herein and optionally, one or more nucleotide sequences encoding one or more additional proteins or non-coding RNAs, or both. In a specific embodiment, provided herein is a host cell comprising two vectors, wherein the first vector comprises a nucleic acid sequence comprising a nucleotide sequence encoding a NPT mutant or a non-naturally occurring NPT, and the second vector comprises a nucleic acid sequence comprising one or more nucleotide sequences encoding one or more additional proteins or one or more non-coding RNAs, or both.

Examples of cells that may be used include those described in this section and in Section 7.5, and Section 8, infra. The cells may be primary cells or cell lines. In a particular embodiment, the host cell is isolated from other cells. In another embodiment, the host cell is not found within the body of a subject. The term “subject” in the context of a cell or body refers to any organism (e.g., bacteria or mammals). The subject may be a human or a non-human mammal.

A NPT mutant or a non-naturally occurring NPT, and optionally one or more additional proteins or one or more non-coding RNAs, or both can be produced by any method known in the art, such as, e.g., by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described in the references cited herein and are fully explained in the literature. See, e.g., Maniatis et al. (1982) Molecular Cloning: A Laboratory Manual Cold Spring Harbor Laboratory Press; Sambrook et al. (1989), Molecular Cloning: A Laboratory Manual Second Edition, Cold Spring Harbor Laboratory Press; Sambrook et al. (2001) Molecular Cloning: A Laboratory Manual Cold Spring Harbor Laboratory Press, Cold Spring Harbor, NY; Ausubel et al, Current Protocols in Molecular Biology, John Wiley & Sons (1987 and annual updates); Current Protocols in Immunology, John Wiley & Sons (1987 and annual updates) Gait (ed.) (1984) Oligonucleotide Synthesis: A Practical Approach, TRL Press; Eckstein (ed.) (1991) Oligonucleotides and Analogues: A Practical Approach, TRL Press; Birren et al. (eds.) (1999) Genome Analysis: A Laboratory Manual Cold Spring Harbor Laboratory Press.

Proteins (e.g., NPT mutants or non-naturally occurring NPT, and optionally a protein of interest) can be prepared using a wide variety of techniques known in the art including recombinant and phage display technologies, or a combination thereof. Examples of phage display methods include those disclosed in Brinkman et al, 1995, J. Immunol. Methods 182:41-50; Ames et al, 1995, J. Immunol. Methods 184: 177-186; Kettleborough et al, 1994, Eur. J. Immunol. 24:952-958; Persic et al, 1997, Gene 187:9-18; Burton et al, 1994, Advances in Immunology 57: 191-280; PCT Application No. PCT/GB91/01 134; International Publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108.

An expression vector can be transferred to a cell (e.g., host cell) by conventional techniques and the resulting cells can then be cultured by conventional techniques to produce a NPT mutant or a non-naturally occurring NPT, and optionally a protein of interest or non-coding RNA can be purified or isolated. A vector (e.g., an expression vector) or nucleic acid sequence or nucleotide sequence can be introduced into a cell (e.g., a host cell) by, e.g., electroporation, transfection, infection, heat shock, microinjection, chromosome transfer, or any or technique known to one of skill in the art.

A variety of host-expression vector systems can be utilized to express a NPT mutant or a non-naturally occurring NPT, and optionally a protein of interest or non-coding RNA. Such host-expression systems represent vehicles by which the coding sequences of interest can be produced and subsequently purified, but also represent cells which can, when transformed or transfected with the appropriate nucleotide coding sequences, express a protein described herein in situ. These include but are not limited to microorganisms such as bacteria (e.g., E. coli and B. subtilis) transformed with recombinant bacteriophage DNA, plasmid DNA or cosmid DNA expression vectors; yeast (e.g., Saccharomyces, Pichia) transformed with recombinant yeast expression vectors; insect cell systems infected with recombinant virus expression vectors (e.g., baculovirus); plant cell systems (e.g., green algae such as Chlamydomonas reinhardtii, or tobacco plants) infected with recombinant virus expression vectors (e.g., cauliflower mosaic virus, CaMV; tobacco mosaic virus, TMV) or transformed with recombinant plasmid expression vectors (e.g., Ti plasmid); or mammalian cell systems (e.g., COS, CHO, BHK, MDCK, HEK 293, NS0, PER.C6, VERO, CRL7030, HsS78Bst, HeLa, and NIH 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

In bacterial systems, a number of expression vectors can be advantageously selected depending upon the use intended for a protein of interest of non-coding RNA expressed. In an insect system, Autographa californica nuclear polyhedrosis virus (AcNPV) may be used as a vector to express foreign genes. The virus grows in Spodoptera frugiperda cells. In mammalian host cells, a number of viral-based expression systems can be utilized. In cases where an adenovirus is used as an expression vector, the protein of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the protein of interest in infected hosts (e.g., see Logan & Shenk, 1984, Proc. Natl. Acad. Sci. USA 8 1:355-359). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bittner et al, 1987, Methods in Enzymol. 153:51-544).

As used herein, the term “host cell” refers to any type of cell, e.g., a primary cell or a cell from a cell line. The host cells may be primary cells, such as fibroblasts, lymphocytes (e.g., B or T cells), epithelial cells, endothelial cells, neurons, astrocytes, hepatocytes, myocytes, chondrocytes, adipocytes, or stem cells (e.g., embryonic stem cells). Alternatively, the host cells may be immortalized cells. In specific embodiments, the term “host cell” refers a cell transfected, infected, microinjected, or transformed a nucleic acid sequence or nucleotide sequence, or otherwise engineered to contain a nucleic acid sequence or nucleotide sequence and the progeny or potential progeny of such a cell. Progeny of such a cell may not be identical to the parent cell transfected with the nucleic acid sequence or nucleotide sequence due to mutations or environmental influences that may occur in succeeding generations or integration of the nucleic acid sequence or nucleotide sequence into the host cell genome.

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, Hela, COS, MDCK, HEK 293, NIH 3T3, W138, BT483, Hs578T, HTB2, BT20 and T47D, NS0 (a murine myeloma cell line), CRL7030 and HsS78Bst cells.

For long-term, high-yield production of recombinant proteins, stable expression is preferred. Rather than using expression vectors that contain viral origins of replication, host cells can be transformed with a nucleic acid sequence (e.g., DNA) controlled by appropriate expression control elements (e.g., promoter, enhancer, sequences, transcription terminators, polyadenylation sites, etc.), and a selectable marker (e.g., a NPT mutant or a non-naturally occurring NPT). Following the introduction of the foreign DNA, engineered cells can be allowed to grow for a certain period of time (e.g., 1-2 days) in an enriched media, and then are switched to a selective media (e.g., media containing an antibiotic, such as neomycin, kanamycin or G418 in the case of a NPT mutant or a non-naturally occurring NPT). The selectable marker in the recombinant plasmid confers resistance to the selection (e.g., neomycin, kanamycin or G418 in the case of a NPT mutant or a non-naturally occurring NPT) and allows cells to stably integrate the plasmid into their chromosomes and grow to form foci which in turn can be cloned and expanded into cell lines. This method can advantageously be used to engineer cell lines which express the protein.

In certain embodiments, provided herein is a method for producing a host cell comprising a second nucleotide sequence, the method comprising (a) introducing a first population of host cells with a first nucleic acid sequence comprising (i) a first nucleotide sequence encoding a NPT mutant or a non-naturally occurring NPT, and (ii) the second nucleotide sequence (e.g., a second nucleotide sequence encoding a second protein or a non-coding RNA); (b) growing the first population of host cells in the presence of kanamycin, neomycin, or G418, or a derivative thereof to produce colonies; and (c) selecting a colony of cells that grows in the presence of the kanamycin, neomycin or G418. In some embodiments, provided herein is a method for producing a host cell comprising a second nucleotide sequence, the method comprising (a) growing a first population of host cells in the presence of kanamycin, neomycin, or G418, or a derivative thereof to produce colonies, wherein a first nucleic acid sequence was introduced into the first population of host cells, and wherein the first nucleic acid sequence comprises (i) a first nucleotide sequence encoding a NPT mutant or a non-naturally occurring NPT, and (ii) the second nucleotide sequence (e.g., a second nucleotide sequence encoding a second protein or a non-coding RNA); and (b) selecting a colony of cells that grows in the presence of the kanamycin, neomycin or G418. In specific embodiments, the first population of host cells produces fewer colonies and/or smaller colonies than a second population of host cells transfected or transformed with a second nucleic acid sequence and grown in the presence of kanamycin, neomycin, or G418, wherein the second nucleic acid sequence comprises a third nucleotide sequence encoding wild-type NPT protein and the second nucleotide sequence. In certain embodiments, the first population of host cells produces 50 to 100, 100 to 1,000, 1,000 to 5,000, 5,000 to 10,000, 1,000 to 10,000, 10,000 to 15,000, 5,000 to 15,000, 15,000 to 25,000, 10,000 to 25,000 times fewer colonies than a second population of host cells transfected or transformed with a second nucleic acid sequence and grown in the presence of kanamycin, neomycin, or G418, wherein the second nucleic acid sequence comprises a third nucleotide sequence encoding wild-type NPT protein and the second nucleotide sequence. In specific embodiments, the first population of host cells comprises a higher copy number of the first nucleic acid sequence as compared to the copy number of a second nucleic acid sequence achieved by a second population of host cells transfected or transformed with the second nucleic acid sequence, wherein the second nucleic acid sequence comprises a third nucleotide sequence encoding wild-type NPT protein and the second nucleotide sequence. In certain embodiments, the first population of host cells comprises a 5 to 10 times, 5 to 15 times, 2 to 5 times, 2 to 10 times, 2 to 15 times, or 10 to 20 times, 10 to 50 times, 10 to 100 times, 50 to 100 times, 50 to 200 times, 50 to 500 times, 100 to 500 times, 100 to 1000 times, 500 to 1000 times, or 2 to 1000 times higher copy number of the first nucleic acid sequence as compared to the copy number of a second nucleic acid sequence achieved by a second population of host cells transfected or transformed with the second nucleic acid sequence, wherein the second nucleic acid sequence comprises a third nucleotide sequence encoding wild-type NPT protein and the second nucleotide sequence. In some embodiments, the first population of host cells comprises at least 2 times, at least 5 times, at least 10 times, at least 15 times, or at least 20 times higher copy number of the first nucleic acid sequence as compared to the copy number of a second nucleic acid sequence achieved by a second population of host cells transfected or transformed with the second nucleic acid sequence, wherein the second nucleic acid sequence comprises a third nucleotide sequence encoding wild-type NPT protein and the second nucleotide sequence. In specific embodiments, the first population of host cells achieves a higher level of expression of the second nucleotide sequence as compared to the level of expression of the second nucleotide sequence achieved by a second population of cells transfected or transformed with a second nucleic acid sequence, wherein the second nucleic acid sequence comprises a third nucleotide sequence encoding wild-type NPT protein and the second nucleotide sequence. In certain embodiments, the first population of host cells achieves a 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher level of expression of the second nucleotide sequence as compared to the level of expression of the second nucleotide sequence achieved by a second population of cells transfected or transformed with a second nucleic acid sequence, wherein the second nucleic acid sequence comprises a third nucleotide sequence encoding wild-type NPT protein and the second nucleotide sequence. In some embodiments, the first population of host cells achieves at least a 5 fold, at least a 10 fold, at least a 25 fold, at least a 50 fold, at least a 100 fold, at least a 200 fold, at least a 250 fold, at least a 500 fold, or at least a 1,000 fold higher level of expression of the second nucleotide sequence as compared to the level of expression of the second nucleotide sequence achieved by a second population of cells transfected or transformed with a second nucleic acid sequence, wherein the second nucleic acid sequence comprises a third nucleotide sequence encoding wild-type NPT protein and the second nucleotide sequence. The copy number can be determined using any technique known in the art (e.g., copy number may be measured using digital droplet PCR to measure the abundance of the second nucleotide sequence relative to a single-copy endogenous gene in the genome of the host cell). The expression of the second nucleotide sequence can be assessed at the RNA level by quantitative reverse transcription PCR (qPCR) or at the protein level by an immunoassay (e.g., a Western blot or immunocytochemistry). In addition, with respect to some proteins encoded by the second nucleotide sequence, the activity (e.g., enzymatic activity) of the protein may be assessed.

In certain embodiments, provided herein is a method for producing a host cell comprising a transgene, comprising (a) introducing a first population of host cells with a first nucleic acid sequence comprising (i) a first nucleotide sequence encoding a NPT mutant or a non-naturally occurring NPT, and (ii) the transgene; (b) growing the first population of host cells in the presence of kanamycin, neomycin, or G418, or a derivative thereof to produce colonies; and (c) selecting a colony of cells that grows in the presence of the kanamycin, neomycin or G418. In some embodiments, provided herein is a method for producing a host cell comprising a transgene, the method comprising (a) growing a first population of host cells in the presence of kanamycin, neomycin, or G418, or a derivative thereof to produce colonies, wherein a first nucleic acid sequence was introduced into the first population of host cells, and wherein the first nucleic acid sequence comprises (i) a first nucleotide sequence encoding a NPT mutant or a non-naturally occurring NPT, and (ii) the transgene; and (b) selecting a colony of cells that grows in the presence of the kanamycin, neomycin or G418. In specific embodiments, the first population of host cells produces fewer colonies and/or smaller colonies than a second population of host cells transfected or transformed with a second nucleic acid sequence and grown in the presence of kanamycin, neomycin, or G418, wherein the second nucleic acid sequence comprises a third nucleotide sequence encoding wild-type NPT protein and the transgene. In certain embodiments, the first population of host cells produces 10 to 100, 100 to 1,000, 1,000 to 5,000, 5,000 to 10,000, 1,000 to 10,000, 10,000 to 15,000, 5,000 to 15,000, 15,000 to 25,000, 10,000 to 25,000 times fewer colonies than a second population of host cells transfected or transformed with a second nucleic acid sequence and grown in the presence of kanamycin, neomycin, or G418, wherein the second nucleic acid sequence comprises a third nucleotide sequence encoding wild-type NPT protein and the transgene. In specific embodiments, the first population of host cells comprises a higher copy number of the first nucleic acid sequence as compared to the copy number of a second nucleic acid sequence achieved by a second population of host cells transfected or transformed with the second nucleic acid sequence, wherein the second nucleic acid sequence comprises a third nucleotide sequence encoding wild-type NPT protein and the transgene. In certain embodiments, the first population of host cells comprises a 5 to 10 times, 5 to 15 times, 2 to 5 times, 2 to 10 times, 2 to 15 times, or 10 to 20 times, 10 to 50 times, 10 to 100 times, 50 to 100 times, 50 to 200 times, 50 to 500 times, 100 to 500 times, 100 to 1000 times, 500 to 1000 times, or 2 to 1000 times higher copy number of the first nucleic acid sequence as compared to the copy number of a second nucleic acid sequence achieved by a second population of host cells transfected or transformed with the second nucleic acid sequence, wherein the second nucleic acid sequence comprises a third nucleotide sequence encoding wild-type NPT protein and the transgene. In some embodiments, the first population of host cells comprises at least 2 times, at least 5 times, at least 10 times, at least 15 times, or at least 20 times higher copy number of the first nucleic acid sequence as compared to the copy number of a second nucleic acid sequence achieved by a second population of host cells transfected or transformed with the second nucleic acid sequence, wherein the second nucleic acid sequence comprises a third nucleotide sequence encoding wild-type NPT protein and transgene. In specific embodiments, the first population of host cells achieves a higher level of expression of the second nucleotide sequence as compared to the level of expression of the second nucleotide sequence achieved by a second population of cells transfected or transformed with a second nucleic acid sequence, wherein the second nucleic acid sequence comprises a third nucleotide sequence encoding wild-type NPT protein and the transgene. In certain embodiments, the first population of host cells achieves a 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher level of expression of the second nucleotide sequence as compared to the level of expression of the second nucleotide sequence achieved by a second population of cells transfected or transformed with a second nucleic acid sequence, wherein the second nucleic acid sequence comprises a third nucleotide sequence encoding wild-type NPT protein and the transgene. In some embodiments, the first population of host cells achieves at least a 10 fold, at least a 25 fold, at least a 50 fold, at least a 100 fold, at least a 200 fold, at least a 250 fold, at least a 500 fold, or at least a 1,000 fold higher level of expression of the transgene as compared to the level of expression of the transgene achieved by a second population of cells transfected or transformed with a second nucleic acid sequence, wherein the second nucleic acid sequence comprises a third nucleotide sequence encoding wild-type NPT protein and the transgene. The copy number can be determined using any technique known in the art (e.g., copy number may be measured using digital droplet PCR to measure the abundance of the transgene relative to a single-copy endogenous gene in the genome of the host cell). The expression of the transgene can be assessed at the RNA level by quantitative reverse transcription PCR (qPCR) or at the protein level by an immunoassay (e.g., a Western blot or immunocytochemistry). In addition, with respect to some proteins encoded by the transgene, the activity (e.g., enzymatic activity) of the protein may be assessed.

In certain embodiments, provided herein is a method for producing a host cell comprising a second nucleotide sequence, the method comprising (a) introducing a first population of host cells with (1) a first nucleic acid sequence comprising a first nucleotide sequence encoding a NPT mutant or a non-naturally occurring NPT, and (2) a second nucleic acid sequence comprising the second nucleotide sequence (e.g., a second nucleotide sequence encoding a second protein or a non-coding RNA); (b) growing the first population of host cells in the presence of a neomycin phosphotransferase substrate (e.g., kanamycin, neomycin, or G418, or a derivative thereof) to produce colonies; and (c) selecting a colony of cells that grows in the presence of the neomycin phosphotransferase substrate (e.g., kanamycin, neomycin, or G418, or a derivative thereof). In some embodiments, provided herein is a method for producing a host cell comprising a second nucleotide sequence, the method comprising (a) growing a first population of host cells in the presence of a neomycin phosphotransferase substrate (e.g., kanamycin, neomycin, or G418, or a derivative thereof) to produce colonies, wherein a first nucleic acid sequence and a second nucleic acid sequence were introduced into the first population of host cells, and wherein the first nucleic acid sequence comprises a first nucleotide sequence encoding a NPT mutant or a non-naturally occurring NPT, and the second nucleic acid sequence comprises the second nucleotide sequence (e.g., a second nucleotide sequence encoding a second protein or a non-coding RNA); and (b) selecting a colony of cells that grows in the presence of the neomycin phosphotransferase substrate (e.g., kanamycin, neomycin, or G418, or a derivative thereof). In specific embodiments, the first population of host cells produces fewer colonies and/or smaller colonies than a second population of host cells transfected or transformed with a third nucleic acid sequence and a fourth nucleic acid sequence, and grown in the presence of the neomycin phosphotransferase substrate (e.g., kanamycin, neomycin, or G418, or a derivative thereof), wherein the third nucleic acid sequence comprises a third nucleotide sequence encoding wild-type NPT protein, and wherein the fourth nucleic acid sequence comprise the second nucleotide sequence. In certain embodiments, the first population of host cells produces 50 to 100, 100 to 1,000, 1,000 to 5,000, 5,000 to 10,000, 1,000 to 10,000, 10,000 to 15,000, 5,000 to 15,000, 15,000 to 25,000, 10,000 to 25,000 times fewer colonies than a second population of host cells transfected or transformed with a third nucleic acid sequence and a fourth nucleic acid sequence, and grown in the presence of the neomycin phosphotransferase substrate (e.g., kanamycin, neomycin, or G418, or a derivative thereof), wherein the third nucleic acid sequence comprises a third nucleotide sequence encoding wild-type NPT protein, and wherein the fourth nucleic acid sequence comprises the second nucleotide sequence. In specific embodiments, the first population of host cells comprises a higher copy number of the first nucleic acid sequence and/or second nucleic acid sequences as compared to the copy number of a third nucleic acid sequence and/or fourth nucleic acid sequence achieved by a second population of host cells transfected or transformed with the third and fourth nucleic acid sequences, wherein the third nucleic acid sequence comprises a third nucleotide sequence encoding wild-type NPT protein, and wherein the fourth nucleic acid sequence comprises the second nucleotide sequence. In certain embodiments, the first population of host cells comprises a 5 to 10 times, 5 to 15 times, 2 to 5 times, 2 to 10 times, 2 to 15 times, or 10 to 20 times, 10 to 50 times, 10 to 100 times, 50 to 100 times, 50 to 200 times, 50 to 500 times, 100 to 500 times, 100 to 1000 times, 500 to 1000 times, or 2 to 1000 times higher copy number of the first nucleic acid sequence and/or second nucleic acid sequence as compared to the copy number of a third nucleic acid sequence and/or fourth nucleic acid sequence achieved by a second population of host cells transfected or transformed with the third and fourth nucleic acid sequences, wherein the third nucleic acid sequence comprises a third nucleotide sequence encoding wild-type NPT protein, and wherein the fourth nucleic acid sequence comprises the second nucleotide sequence. In some embodiments, the first population of host cells comprises at least 2 times, at least 5 times, at least 10 times, at least 15 times, or at least 20 times, at least 50 times, at least 100 times, at least 200 times, at least 500 times, or at least 1000 times higher copy number of the first nucleic acid sequence and/or second nucleic acid sequences as compared to the copy number of a third nucleic acid sequence and/or a fourth nucleic acid sequence achieved by a second population of host cells transfected or transformed with the third and fourth nucleic acid sequences, wherein the third nucleic acid sequence comprises a third nucleotide sequence encoding wild-type NPT protein, and wherein the fourth nucleic acid sequence comprises the second nucleotide sequence. In specific embodiments, the first population of host cells achieves a higher level of expression of the second nucleotide sequence as compared to the level of expression of the second nucleotide sequence achieved by a second population of cells transfected or transformed with a third nucleic acid sequence and a fourth nucleic acid sequence, wherein the third nucleic acid sequence comprises a third nucleotide sequence encoding wild-type NPT protein, and wherein the fourth nucleic acid sequence comprises the second nucleotide sequence. In certain embodiments, the first population of host cells achieves a 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher level of expression of the second nucleotide sequence as compared to the level of expression of the second nucleotide sequence achieved by a second population of cells transfected or transformed with a third nucleic acid sequence and a fourth nucleic acid sequence, wherein the third nucleic acid sequence comprises a third nucleotide sequence encoding wild-type NPT protein, and wherein the fourth nucleic acid sequence comprises the second nucleotide sequence. In some embodiments, the first population of host cells achieves at least a 10 fold, at least a 25 fold, at least a 50 fold, at least a 100 fold, at least a 200 fold, at least a 250 fold, at least a 500 fold, or at least a 1,000 fold higher level of expression of the second nucleotide sequence as compared to the level of expression of the second nucleotide sequence achieved by a second population of cells transfected or transformed with a third nucleic acid sequence and a fourth nucleic acid sequence, wherein the third nucleic acid sequence comprises a third nucleotide sequence encoding wild-type NPT protein, and wherein the fourth nucleic acid sequence comprises the second nucleotide sequence. The copy number can be determined using any technique known in the art (e.g., copy number may be measured using digital droplet PCR to measure the abundance of the second nucleotide sequence relative to a single-copy endogenous gene in the genome of the host cell). The expression of the second nucleotide sequence can be assessed at the RNA level by quantitative reverse transcription PCR (qPCR) or at the protein level by an immunoassay (e.g., a Western blot or immunocytochemistry). In addition, with respect to some proteins encoded by the second nucleotide sequence, the activity (e.g., enzymatic activity) of the protein may be assessed.

In certain embodiments, provided herein is a method for producing a host cell comprising a transgene, the method comprising (a) introducing a first population of host cells with (1) a first nucleic acid sequence comprising a first nucleotide sequence encoding a NPT mutant or a non-naturally occurring NPT, and (2) a second nucleic acid sequence comprising the transgene (e.g., a transgene encoding a second protein or a non-coding RNA); (b) growing the first population of host cells in the presence of a neomycin phosphotransferase substrate (e.g., kanamycin, neomycin, or G418, or a derivative thereof) to produce colonies; and (c) selecting a colony of cells that grows in the presence of the neomycin phosphotransferase substrate (e.g., kanamycin, neomycin, or G418, or a derivative thereof). In some embodiments, provided herein is a method for producing a host cell comprising a transgene, the method comprising (a) growing a first population of host cells in the presence of a neomycin phosphotransferase substrate (e.g., kanamycin, neomycin, or G418, or a derivative thereof) to produce colonies, wherein a first nucleic acid sequence and a second nucleic acid sequence were introduced into the first population of host cells, and wherein the first nucleic acid sequence comprises a first nucleotide sequence encoding a NPT mutant or a non-naturally occurring NPT, and the second nucleic acid sequence comprises the transgene (e.g., a transgene encoding a second protein or a non-coding RNA); and (b) selecting a colony of cells that grows in the presence of the neomycin phosphotransferase substrate (e.g., kanamycin, neomycin, or G418, or a derivative thereof). In specific embodiments, the first population of host cells produces fewer colonies than a second population of host cells transfected or transformed with a third nucleic acid sequence and a fourth nucleic acid sequence, and grown in the presence of the neomycin phosphotransferase substrate (e.g., kanamycin, neomycin, or G418, or a derivative thereof), wherein the third nucleic acid sequence comprises a third nucleotide sequence encoding wild-type NPT protein, and wherein the fourth nucleic acid sequence comprise the transgene. In certain embodiments, the first population of host cells produces 100 to 1,000, 1,000 to 5,000, 5,000 to 10,000, 1,000 to 10,000, 10,000 to 15,000, 5,000 to 15,000, 15,000 to 25,000, 10,000 to 25,000 times fewer colonies than a second population of host cells transfected or transformed with a third nucleic acid sequence and a fourth nucleic acid sequence, and grown in the presence of the neomycin phosphotransferase substrate (e.g., kanamycin, neomycin, or G418, or a derivative thereof), wherein the third nucleic acid sequence comprises a third nucleotide sequence encoding wild-type NPT protein, and wherein the fourth nucleic acid sequence comprises the transgene. In specific embodiments, the first population of host cells comprises a higher copy number of the first nucleic acid sequence and/or second nucleic acid sequences as compared to the copy number of a third nucleic acid sequence and/or fourth nucleic acid sequence achieved by a second population of host cells transfected or transformed with the third and fourth nucleic acid sequences, wherein the third nucleic acid sequence comprises a third nucleotide sequence encoding wild-type NPT protein, and wherein the fourth nucleic acid sequence comprises the transgene. In certain embodiments, the first population of host cells comprises a 5 to 10 times, 5 to 15 times, 2 to 5 times, 2 to 10 times, 2 to 15 times, or 10 to 20 times, 10 to 50 times, 10 to 100 times, 50 to 100 times, 50 to 200 times, 50 to 500 times, 100 to 500 times, 100 to 1000 times, 500 to 1000 times, or 2 to 1000 times higher copy number of the first nucleic acid sequence and/or second nucleic acid sequence as compared to the copy number of a third nucleic acid sequence and/or fourth nucleic acid sequence achieved by a second population of host cells transfected or transformed with the third and fourth nucleic acid sequences, wherein the third nucleic acid sequence comprises a third nucleotide sequence encoding wild-type NPT protein, and wherein the fourth nucleic acid sequence comprises the transgene. In some embodiments, the first population of host cells comprises at least 2 times, at least 5 times, at least 10 times, at least 15 times, or at least 20 times, at least 50 times, at least 100 times, at least 200 times, at least 500 times, or at least 1000 times higher copy number of the first nucleic acid sequence and/or second nucleic acid sequences as compared to the copy number of a third nucleic acid sequence and/or a fourth nucleic acid sequence achieved by a second population of host cells transfected or transformed with the third and fourth nucleic acid sequences, wherein the third nucleic acid sequence comprises a third nucleotide sequence encoding wild-type NPT protein, and wherein the fourth nucleic acid sequence comprises the transgene. In specific embodiments, the first population of host cells achieves a higher level of expression of the transgene as compared to the level of expression of the transgene achieved by a second population of cells transfected or transformed with a third nucleic acid sequence and a fourth nucleic acid sequence, wherein the third nucleic acid sequence comprises a third nucleotide sequence encoding wild-type NPT protein, and wherein the fourth nucleic acid sequence comprises the transgene. In certain embodiments, the first population of host cells achieves a 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher level of expression of the transgene as compared to the level of expression of the transgene achieved by a second population of cells transfected or transformed with a third nucleic acid sequence and a fourth nucleic acid sequence, wherein the third nucleic acid sequence comprises a third nucleotide sequence encoding wild-type NPT protein, and wherein the fourth nucleic acid sequence comprises the transgene. In some embodiments, the first population of host cells achieves at least a 10 fold, at least a 25 fold, at least a 50 fold, at least a 100 fold, at least a 200 fold, at least a 250 fold, at least a 500 fold, or at least a 1,000 fold higher level of expression of the transgene as compared to the level of expression of the transgene achieved by a second population of cells transfected or transformed with a third nucleic acid sequence and a fourth nucleic acid sequence, wherein the third nucleic acid sequence comprises a third nucleotide sequence encoding wild-type NPT protein, and wherein the fourth nucleic acid sequence comprises the transgene. The copy number can be determined using any technique known in the art (e.g., copy number may be measured using digital droplet PCR to measure the abundance of the transgene relative to a single-copy endogenous gene in the genome of the host cell). The expression of the transgene can be assessed at the RNA level by quantitative reverse transcription PCR (qPCR) or at the protein level by an immunoassay (e.g., a Western blot or immunocytochemistry). In addition, with respect to some proteins encoded by the transgene, the activity (e.g., enzymatic activity) of the protein may be assessed.

In specific embodiments, a NPT mutant or non-naturally occurring NPT is one described in Section 7.1 or 8. In some embodiments, the transgene is one described in Section 7.2.

Methods commonly known in the art of recombinant DNA technology can be routinely applied to select the desired recombinant clone, and such methods are described, for example, in Ausubel et al. (eds.), Current Protocols in Molecular Biology, John Wiley & Sons, N Y (1993); Kriegler, Gene Transfer and Expression, A Laboratory Manual, Stockton Press, N Y (1990); and in Chapters 12 and 13, Dracopoli et al. (eds.), Current Protocols in Human Genetics, John Wiley & Sons, N Y (1994); Colberre-Garapin et al, 1981, J. Mol. Biol. 150: 1, which are incorporated by reference herein in their entireties.

A host cell can be co-transfected with two or more expression vectors described herein. The two vectors can contain identical selectable markers (e.g., a NPT mutant or non-naturally occurring NPT) which enable equal expression of a protein of interest or non-coding RNA. The host cells can be co-transfected with different amounts of the two or more expression vectors. For example, host cells can be transfected with any one of the following ratios of a first expression vector and a second expression vector: 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, a NPT mutant or a non-naturally occurring NPT described herein and a protein of interest or non-coding RNA. The expression vector can be monocistronic or multicistronic. A multicistronic nucleic acid construct can encode 2, 3, 4, 5, 6, 7, 8, 9, 10 or more, or in the range of 2-5, 5-10 or 10-20 genes/nucleotide sequences. For example, a bicistronic nucleic acid construct can comprise in the following order a promoter, a first gene (e.g., a NPT mutant or a non-naturally occurring NPT), and a second gene (e.g., a protein of interest or non-coding RNA). In such an expression vector, the transcription of both genes can be driven by the promoter, whereas the translation of the mRNA from the first gene can be by a cap-dependent scanning mechanism and the translation of the mRNA from the second gene can be by a cap-independent mechanism, e.g., by an IRES.

Once a protein of interest described herein has been produced by recombinant expression, it can be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins. Further, protein of interest can be fused to a heterologous polypeptide sequence known in the art (e.g., a Flag tag or His tag) to facilitate purification.

In specific embodiments, a protein described herein (e.g., a NPT mutant or a non-naturally occurring NPT, or a protein of interest) is isolated or purified. Generally, an isolated protein is one that is substantially free of other proteins than the isolated protein. For example, in a particular embodiment, a preparation of a protein described herein is substantially free of cellular material and/or chemical precursors. The language “substantially free of cellular material” includes preparations of a protein described herein in which the protein is separated from cellular components of the cells from which it is isolated or recombinantly produced. Thus, a protein described herein that is substantially free of cellular material includes preparations of protein having less than about 30%, 20%, 10%, 5%, 2%, 1%, 0.5%, or 0.1% (by dry weight) of heterologous protein (also referred to herein as a “contaminating protein”) and/or variants of a protein, for example, different post-translational modified forms of a protein or other different versions of a protein. When the protein is recombinantly produced, it is also generally substantially free of culture medium, i.e., culture medium represents less than about 20%, 10%, 2%, 1%, 0.5%, or 0.1% of the volume of the protein preparation. When the protein is produced by chemical synthesis, it is generally substantially free of chemical precursors or other chemicals, i.e., it is separated from chemical precursors or other chemicals that are involved in the synthesis of the protein. Accordingly, such preparations of the protein have less than about 30%, 20%, 10%, or 5% (by dry weight) of chemical precursors or compounds other than the protein of interest. In a specific embodiment, proteins described herein are isolated or purified.

### 7.5 Cells

In another aspect, provided herein is a host cell. In certain embodiments, a host cell comprises a vector comprising nucleotide sequence encoding a NPT mutant or a non-naturally occurring NPT. In some embodiments, a host cell comprises a nucleic acid sequence or nucleotide sequence described herein (e.g., in Section 7.2 or Section 8). In a specific embodiment, a host cell comprises a nucleic acid sequence comprising SEQ ID NO:20. In another specific embodiment, a host cell comprises a nucleic acid sequence comprising SEQ ID NO:32. In another specific embodiment, a host cell comprises a nucleic acid sequence comprising SEQ ID NO:33. In another specific embodiment, a host cell comprises a nucleic acid sequence comprising SEQ ID NO:34. In another specific embodiment, a host cell comprises a nucleic acid sequence comprising SEQ ID NO:36. In another specific embodiment, a host cell comprises a nucleic acid sequence comprising SEQ ID NO:37.

In some embodiments, a host cell comprises a NPT mutant or a non-naturally occurring NPT described herein (e.g., Section 7.1 or Section 8). In certain embodiments, a host cell expresses a NPT mutant or a non-naturally occurring NPT described herein (e.g., Section 7.1 or Section 8).

Any host cell described herein (e.g., Section 7.4 or Section 8) or known to those skilled in the art in view of the present disclosure can be used for recombinant expression of a NPT mutant or a non-naturally occurring NPT described herein (e.g., Section 7.1 or Section 8). For instance, such host cells can be cultured and made to co-express a NPT mutant or a non-naturally occurring NPT and a transgene when a nucleic acid sequence encoding the NPT mutant or the non-naturally occurring NPT and transgene are introduced into the cell. See, e.g., Section 7.4 and Section 8 for examples of host cells.

In certain embodiments, a cell (e.g., host cell) is an in vitro or ex vivo cell. In certain embodiments, a host cell is isolated from cells not transfected or transformed by a nucleotide sequence encoding a NPT mutant or a non-naturally occurring NPT. A host cell can be any type of cell described herein or known in the art.

In some embodiments, a host cell is a bacterial or a eukaryotic cell. In certain embodiments, a host cell is a yeast, insect, mammalian or plant cell. In embodiments where the host cell is a bacterial cell, the cell is an E. coli cell. Exemplary E. coli cells can be, for instance, E. coli TG1 or BL21 cell, but are not restricted thereto.

In some embodiments, a host cell is a mammalian cell. In certain embodiments, a host cell is a from a human cell line. Suitable mammalian cells include, for instance, CHO and HEK239 cells, and variants thereof (e.g., CHO-DG44 or CHO-K1 cells).

In certain embodiments, a host cell is an immortalized cell line. In some embodiments, a host cell is a HEK293, CHO, PER.C6, murine NS0 cell, fibrosarcoma HT-1080 cell, murine Sp2/0 cell, BHK cell, or a murine C127 cell.

In specific embodiments, a host cell is a primary cell, such as, for instance, and without limitation thereto, a fibroblast or blood cell (e.g., B cell or T cell). In some embodiments, a host cell is an embryonic stem cell.

In some embodiments, a host cell is an insect cell. In certain embodiments, a host cell is a plant cell.

Cultured immortalized cells can be transfected with nucleic acid encoding NPT mutant or a non-naturally occurring NPT for short term (transiently), or long term (stable) expression, depending on whether the nucleic acid introduced into the cell is integrated into the host cell genome. Transient DNA expression typically lasts 24-72 hours, whereas stable DNA expression potentially allows permanent overexpression of the protein.

According to particular embodiments, a recombinant expression vector is introduced into host cells by conventional methods such as chemical transfection, heat shock, or electroporation, such that the recombinant nucleic acid sequence is effectively expressed.

In certain embodiments, a nucleic acid sequence or nucleotide sequence encoding a NPT mutant or non-naturally occurring NPT described herein is stably integrated into the genome of a cell (e.g., host cell). The nucleic acid sequence or nucleotide sequence may be randomly integrated into the genome of a cell (e.g., host cell). Alternatively, the nucleic acid sequence or nucleotide sequence may be integrated into the genome of a cell (e.g., host cell) at specific locations. Multiple copies of the nucleic acid sequence or nucleotide sequence may be integrated into the genome of a cell. (e.g., host cell). For example, a host cell may contain 5, 10, 15, 20, 25 or more copies of the nucleic acid sequence or nucleotide sequence integrated into its genome. In some embodiments, the transgene is one described herein (e.g., in Section 7.2).

In some embodiments, a host cell is a mammalian cell, and a nucleic acid sequence or nucleotide sequence encoding the NPT mutant or non-naturally occurring NPT and optionally, a transgene is introduced into the cell by transfection, transduction, infection, microinjection or chromosome transfer.

In some embodiments, the second nucleotide sequence encodes a protein of interest or a non-coding RNA described herein (e.g., Section 7.2).

In specific embodiments, a first population of host cells transformed or transfected with a first nucleic acid sequence comprises a higher copy number of the first nucleic acid sequence as compared to the copy number of a second nucleic acid sequence achieved by a second population of host cells transfected or transformed with the second nucleic acid sequence, wherein the first population of cells comprises the first nucleic acid sequence comprising a first nucleotide sequence encoding a NPT mutant or a non-naturally occurring NPT and a second nucleotide sequence (e.g., a second nucleotide sequence encoding a protein of interest or a non-coding RNA), and wherein the second nucleic acid sequence comprises a third nucleotide sequence encoding wild-type NPT protein and the second nucleotide sequence. In some embodiments, a first population of host cells transformed or transfected with a first nucleic acid sequence comprises at least 2 times, at least 5 times, at least 10 times, at least 15 times, or at least 20 times, at least 50 times, at least 100 times, at least 200 times, at least 500 times, or at least 1000 times higher copy number of the first nucleic acid sequence as compared to the copy number of a second nucleic acid sequence achieved by a second population of host cells transfected or transformed with the second nucleic acid sequence, wherein the first population of cells comprises the first nucleic acid sequence comprising a first nucleotide sequence encoding a NPT mutant or a non-naturally occurring NPT and a second nucleotide sequence (e.g., a second nucleotide sequence encoding a protein of interest or a non-coding RNA), and wherein the second nucleic acid sequence comprises a third nucleotide sequence encoding wild-type NPT protein and the second nucleotide sequence. In some embodiments, a first population of host cells transformed or transfected with a first nucleic acid sequence comprises 2 to 20 times, 2 to 100 times, 2 to 500 times, 2 to 1000 times, 50 to 100 times, 50 to 500 times, 50 to 1000 times, or 500 to 1000 times higher copy number of the first nucleic acid sequence as compared to the copy number of a second nucleic acid sequence achieved by a second population of host cells transfected or transformed with the second nucleic acid sequence, wherein the first population of cells comprises the first nucleic acid sequence comprising a first nucleotide sequence encoding a NPT mutant or a non-naturally occurring NPT and a second nucleotide sequence (e.g., a second nucleotide sequence encoding a protein of interest or a non-coding RNA), and wherein the second nucleic acid sequence comprises a third nucleotide sequence encoding wild-type NPT protein and the second nucleotide sequence. In specific embodiments, a first population of host cells transformed or transfected with a first nucleic acid sequence achieves a higher level of expression of a second nucleotide sequence as compared to the level of expression of the second nucleotide sequence achieved by a second population of cells transfected or transformed with a second nucleic acid sequence, wherein the first population of cells comprises the first nucleic acid sequence comprising a first nucleotide sequence encoding a NPT mutant or a non-naturally occurring NPT and a second nucleotide sequence (e.g., a second nucleotide sequence encoding a protein of interest or a non-coding RNA), and wherein the second nucleic acid sequence comprises a third nucleotide sequence encoding wild-type NPT protein and the second nucleotide sequence. In certain embodiments, a first population of host cells transformed or transfected with a first nucleic acid sequence achieves a 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher level of expression of a second nucleotide sequence as compared to the level of expression of the second nucleotide sequence achieved by a second population of cells transfected or transformed with a second nucleic acid sequence, wherein the first population of cells comprises the first nucleic acid sequence comprising a first nucleotide sequence encoding a NPT mutant or a non-naturally occurring NPT and a second nucleotide sequence (e.g., a second nucleotide sequence encoding a protein of interest or a non-coding RNA), and wherein the second nucleic acid sequence comprises a third nucleotide sequence encoding wild-type NPT protein and the second nucleotide sequence. In some embodiments, a first population of host cells transformed or transfected with a first nucleic acid sequence achieves at least a 10 fold, at least a 25 fold, at least a 50 fold, at least a 100 fold, at least a 200 fold, at least a 250 fold, at least a 500 fold, or at least a 1,000 fold higher level of expression of a second nucleotide sequence as compared to the level of expression of the second nucleotide sequence achieved by a second population of cells transfected or transformed with a second nucleic acid sequence, wherein the first population of cells comprises the first nucleic acid sequence comprising a first nucleotide sequence encoding a NPT mutant or a non-naturally occurring NPT and a second nucleotide sequence (e.g., a second nucleotide sequence encoding a protein of interest or a non-coding RNA), and wherein the second nucleic acid sequence comprises a third nucleotide sequence encoding wild-type NPT protein and the second nucleotide sequence. The copy number can be determined using any technique known in the art (e.g., copy number may be measured using digital droplet PCR to measure the abundance of the second nucleotide sequence relative to a single-copy endogenous gene in the genome of the host cell). The expression of the second nucleotide sequence can be assessed at the RNA level by quantitative reverse transcription PCR (qPCR) or at the protein level by an immunoassay (e.g., a Western blot or immunocytochemistry). In addition, with respect to some proteins encoded by the second nucleotide sequence, the activity (e.g., enzymatic activity) of the protein may be assessed.

In specific embodiments, a first population of host cells transformed or transfected with a first nucleic acid sequence comprises a higher copy number of the first nucleic acid sequence as compared to the copy number of a second nucleic acid sequence achieved by a second population of host cells transfected or transformed with the second nucleic acid sequence, wherein the first population of cells comprises the first nucleic acid sequence comprising a first nucleotide sequence encoding a NPT mutant or a non-naturally occurring NPT and a transgene, and wherein the second nucleic acid sequence comprises a third nucleotide sequence encoding wild-type NPT protein and the transgene. In some embodiments, a first population of host cells transformed or transfected with a first nucleic acid sequence comprises at least 2 times, at least 5 times, at least 10 times, at least 15 times, or at least 20 times, at least 50 times, at least 100 times, at least 200 times, at least 500 times, or at least 1000 times higher copy number of the first nucleic acid sequence as compared to the copy number of a second nucleic acid sequence achieved by a second population of host cells transfected or transformed with the second nucleic acid sequence, wherein the first population of cells comprises the first nucleic acid sequence comprising a first nucleotide sequence encoding a NPT mutant or a non-naturally occurring NPT and a transgene, and wherein the second nucleic acid sequence comprises a third nucleotide sequence encoding wild-type NPT protein and the transgene. In some embodiments, a first population of host cells transformed or transfected with a first nucleic acid sequence comprises 2 to 20 times, 2 to 100 times, 2 to 500 times, 2 to 1000 times, 50 to 100 times, 50 to 500 times, 50 to 1000 times, or 500 to 1000 times higher copy number of the first nucleic acid sequence as compared to the copy number of a second nucleic acid sequence achieved by a second population of host cells transfected or transformed with the second nucleic acid sequence, wherein the first population of cells comprises the first nucleic acid sequence comprising a first nucleotide sequence encoding a NPT mutant or a non-naturally occurring NPT and a transgene, and wherein the second nucleic acid sequence comprises a third nucleotide sequence encoding wild-type NPT protein and the transgene. In specific embodiments, a first population of host cells transformed or transfected with a first nucleic acid sequence achieves a higher level of expression of a transgene as compared to the level of expression of the transgene achieved by a second population of cells transfected or transformed with a second nucleic acid sequence, wherein the first population of cells comprises the first nucleic acid sequence comprising a first nucleotide sequence encoding a NPT mutant or a non-naturally occurring NPT and the transgene, and wherein the second nucleic acid sequence comprises a third nucleotide sequence encoding wild-type NPT protein and the transgene. In certain embodiments, a first population of host cells transformed or transfected with a first nucleic acid sequence achieves a 5 to 25 fold, 10 to 25 fold, 10 to 50 fold, 10 to 100 fold, 50 to 100 fold, 50 to 200 fold, 50 to 500 fold, 100 to 500 fold, 100 to 1000 fold, 500 to 1,000 fold, or 5 to 1000 fold higher level of expression of a transgene as compared to the level of expression of the transgene achieved by a second population of cells transfected or transformed with a second nucleic acid sequence, wherein the first population of cells comprises the first nucleic acid sequence comprising a first nucleotide sequence encoding a NPT mutant or a non-naturally occurring NPT and the transgene, and wherein the second nucleic acid sequence comprises a third nucleotide sequence encoding wild-type NPT protein and the transgene. In some embodiments, a first population of host cells transformed or transfected with a first nucleic acid sequence achieves at least a 10 fold, at least a 25 fold, at least a 50 fold, at least a 100 fold, at least a 200 fold, at least a 250 fold, at least a 500 fold, or at least a 1,000 fold higher level of expression of a transgene as compared to the level of expression of the transgene achieved by a second population of cells transfected or transformed with a second nucleic acid sequence, wherein the first population of cells comprises the first nucleic acid sequence comprising a first nucleotide sequence encoding a NPT mutant or a non-naturally occurring NPT and the transgene, and wherein the second nucleic acid sequence comprises a third nucleotide sequence encoding wild-type NPT protein and the transgene. The copy number can be determined using any technique known in the art (e.g., copy number may be measured using digital droplet PCR to measure the abundance of the transgene relative to a single-copy endogenous gene in the genome of the host cell). The expression of the transgene can be assessed at the RNA level by quantitative reverse transcription PCR (qPCR) or at the protein level by an immunoassay (e.g., a Western blot or immunocytochemistry). In addition, with respect to some proteins encoded by the transgene, the activity (e.g., enzymatic activity) of the protein may be assessed.

In some embodiments, the transgene is one described herein (e.g., in Section 7.2). In some embodiments, a NPT mutant or a non-naturally occurring NPT is one described herein (e.g., in Section 7.1 or Section 8).

In certain embodiments, a host cell is virus cell producer cell line containing nucleic acid sequence or nucleotide sequence encoding a NPT mutant or a non-naturally occurring NPT described herein. The viral producer cell line may express a capsid protein or other surface protein (e.g., envelope protein), a protein required for replication, or both. Suitable virus producer cell lines can be for AAV, adenovirus, retrovirus, lentivirus, herpes simplex virus, vaccinia virus, or baculovirus. The virus producer cell line may be used to produce virus for, e.g., gene therapy or vaccination purposes.

In a specific embodiment, provided herein is a virus producer cell line comprising one or more nucleic acid sequences, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and (ii) a second nucleic acid sequence encoding one or more viral proteins, wherein the one or more viral proteins are a capsid protein or envelope protein, a viral protein necessary for replication, or both.

In some embodiments, the virus producer cell line comprises a NPT mutant nucleic acid sequence of any one of SEQ ID NOS: 20, 32, 33, 34, 36, or 37.

In certain embodiments of a virus producer cell line provided herein, the encoded one or more viral proteins can be, for instance, an AAV capsid protein, an AAV rep protein, an adenovirus E1 region proteins required for adenovirus replication, a retroviral envelope protein, a retroviral gag protein, or a retroviral reverse transcriptase, or a combination thereof. For instance, the one or more viral proteins can be a retroviral envelope protein, gag protein and reverse transcriptase.

In another embodiment, provided herein is an antigen producing cell line comprising one or more nucleic acid sequences, wherein the one or more nucleic acid sequences comprise: (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with: (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 210 of SEQ ID NO:1 is a substitution to alanine; (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid; (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine; (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine; (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and (ii) a second nucleic acid sequence encoding one or more antigens.

In some embodiments, the antigen producing cell line comprises a NPT mutant nucleic acid sequence of any one of SEQ ID NOS: 20, 32, 33, 34, 36, and 37.

In certain embodiments, an antigen producing cell line comprises a nucleic acid sequence encoding a viral antigen, a bacterial antigen, or a fungal antigen. In other embodiments, an antigen producing cell line comprises a nucleic acid sequence encoding a cancer antigen.

In certain embodiments, provided herein is an in vitro or ex vivo cell expressing a non-naturally occurring NPT, wherein the non-naturally occurring NPT is attenuated relative to a wild-type neomycin phosphotransferase, and wherein the non-naturally occurring NPT comprises an amino acid sequence of any one of SEQ ID NOS: 38, 39, 40, 41, 42, and 43.

In certain embodiments wherein the host cell is a bacterial cell transfected or transformed with a nucleotide sequence encoding a NPT mutant or non-naturally occurring NPT as provided herein, the bacterial cell exhibits reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding a wild-type NPT.

In certain embodiments wherein the host cell is a mammalian cell transfected a nucleotide sequence encoding a NPT mutant or non-naturally occurring NPT as provided herein, the mammalian cell exhibits reduced colony formation as assessed by a colony formation assay after 2 weeks growth on tissue-culture plates in media containing 500 μg/mL geneticin (G418) relative to mammalian cells transfected with a nucleotide sequence encoding wild-type NPT.

In certain embodiments, a host cell comprises first nucleic acid sequence encoding a NPT mutant or a non-naturally occurring NPT, and a second nucleic acid sequence encoding a second protein or a non-coding RNA.

In some embodiments, the second protein or non-coding RNA is one described herein (e.g., in Section 7.2). In some embodiments, a host cell or population of host cells is produced by a method described herein (e.g., in Section 7.4 or Section 8).

### 7.6 Methods of Use

In a specific embodiment, a NPT mutant or a non-naturally occurring NPT described herein, or a nucleotide sequence encoding a NPT mutant or a non-naturally occurring NPT described herein is used any way one of skill in the art would use wild-type NPT. In specific embodiments, a NPT mutant or a non-naturally occurring NPT described herein, or a nucleotide sequence encoding a NPT mutant or a non-naturally occurring NPT described herein is used in any way a selectable marker would be used by a person skilled in the art. In certain embodiments, a NPT mutant or a non-naturally occurring NPT described herein, or a nucleotide sequence encoding a NPT mutant or a non-naturally occurring NPT described herein is used as described herein.

In specific embodiments, a neomycin phosphotransferase substrate (e.g., kanamycin, neomycin, or G418, or a derivative thereof) is used to select for host cells (e.g., mammalian host cells) transformed or transfected with a nucleic acid sequence comprising a nucleotide sequence encoding a NPT mutant or a non-naturally occurring NPT described herein and an exogenous sequence(s), which host cells have the exogenous sequence(s) stably integrated into chromosomes. Transfection, transduction, infection, microinjection or chromosome transfer may be used to introduce the nucleic acid sequence into the host cells. This methodology could be used to express a protein of interest or to disrupt a gene by insertional mutagenesis (e.g., by inserting DNA by homologous recombination or by transposon insertion).

In specific embodiments, host cells that carry stable episomes (non-integrated plasmids that replicate such as those that contain EBNA1 OriP sequences and express EBNA1 and a NPT mutant or a non-naturally occurring NPT described herein) at high numbers may be selected using, e.g., neomycin, kanamycin or G418. In certain embodiments, a high copy number is 5 to 10 times, 5 to 15 times, 2 to 5 times, 2 to 10 times, 2 to 15 times, or 10 to 20 times, 10 to 50 times, 10 to 100 times, 50 to 100 times, 50 to 200 times, 50 to 500 times, 100 to 500 times, 100 to 1000 times, 500 to 1000 times, or 2 to 1000 times higher than achieved when a nucleotide sequence encoding wild-type NPT is used in place of the NPT mutant or non-naturally occurring NPT.

In specific embodiments, short-term culture of host cells (e.g., mammalian cells) with a neomycin phosphotransferase substrate (e.g., kanamycin, neomycin, or G418, or a derivative thereof) can be used to enrich for cells that received constructs expressing a NPT mutant or a non-naturally occurring NPT described herein as well as other co-transfected nucleic acid sequences (e.g., DNA or RNA) encoding a protein or non-coding RNA wherein the construct expressing the NPT mutant or non-naturally occurring NPT is not integrated. For example, some cells are difficult to transfect and enriching for cells that received and expressed the NPT gene can also enrich for cells that received co-transfected Crispr constructs, hence decreasing the screening need to identify cells with the desired modification (e.g., gene knockout).

In specific embodiments, host cells engineered to express a NPT mutant or a non-naturally occurring NPT described herein may be used to select for those host cells that have undergone gene amplification using, e.g., neomycin, kanamycin, G418 or a derivative thereof. For example, inhibitors of DHFR may be used in this way to “amplify” chromosomal regions that contain integrated transgenes in host cells (e.g., mammalian cells, such as CHO cells).

In specific embodiments, a nucleotide sequence encoding a NPT mutant or a non-naturally occurring NPT described herein may be used as a selection gene when creating cell lines by chromosome transfer such as in the creation of Human Hamster Hybrids or transfer of chromosomes between cells by cell fusion.

In specific embodiments, embryonic stem cells are engineered to contain a nucleotide sequence encoding a NPT mutant or a non-naturally occurring NPT described herein and the npt gene is introduced into the chromosome during homologous recombination in the embryonic stem cells (creating a heterozygous insertion), higher concentrations of G418 may be used in order to select for rare cells that have inherited 2 knockout chromosomes by nondisjunction. This would allow some analyses of the knockout phenotype by characterization of the cells in vitro or in vivo without first having to introduce the cells into mice, and breeding the mice to generate homozygotes.

In specific embodiments, highly active gene promoters in host cells could be identified by genome-wide screening using transposons engineered with a promoter-less NPT mutant nucleotide gene or a non-naturally occurring NPT gene placed downstream of a splice acceptor. Transposons that insert into genes with very active promoters that activate NPT expression can be selected using the appropriate level of neomycin phosphotransferase substrate (e.g., neomycin, kanamycin, or G418, or a derivative thereof). The identity of the relevant genes and promoters can be subsequently identified by characterizing the transposon insertion sites in the surviving cells.

In specific embodiments, host cells (e.g., bacteria) transformed with a first nucleotide sequence encoding a NPT mutant or a non-naturally occurring NPT and one or more covalently linked additional nucleotide sequences may be selected by culturing cells with the appropriate neomycin phosphotransferase substrate (e.g., neomycin, kanamycin, or G418, or a derivative thereof). The nucleotide sequences encoding the NPT gene may be present in a cloning vector, virus or in genomic insertion in the host cells.

In specific embodiments, plasmids comprising a nucleotide sequence encoding a NPT mutant or non-naturally occurring NPT that is only expressed in bacteria may be used to create gene therapy products, including, for example, a lentivirus or AAV. The highly attenuated nature of the NPT mutant or non-naturally occurring NPT makes any aberrant packaging of the nucleotide sequence encoding a NPT mutant or non-naturally occurring NPT and delivery to patients much safer since the gene is much less active.

In specific embodiments, concatamers of DNAs may be created, such as by ligating a linear fragment containing a gene of interest and the nucleotide sequence encoding the NPT mutant or non-naturally occurring NPT to a fragment with a bacterial replication origin, transforming host cells and selecting using, e.g., neomycin, kanamycin, or G418, or a derivative thereof, for surviving cells which have multiple copies of the gene ligated together. This may be used to generate a head-to-tail array of genes that can be delivered to mammalian host cells and can result in a higher frequency of multicopy insertions into the host chromosomes.

In specific embodiments, a nucleotide sequence encoding a NPT mutant or a non-naturally occurring NPT may be used anywhere where G418 and other NPT substrates are toxic to cells (e.g., yeast, bacteria, insect cells, animal cells, plants and any pathogens of those organisms).

In some embodiments, a nucleotide sequence encoding a NPT mutant or non-naturally occurring NPT is used as described in Section 8.

### 7.7 Kits

In another aspect, provided herein are kits. In one embodiments, a kit provided herein comprises, in a container, a nucleic acid sequence comprising a nucleotide sequence encoding a NPT mutant or a non-naturally occurring NPT. In another embodiment, a kit provided herein comprises, in a container, a vector (e.g., an expression vector) comprising a nucleic acid sequence or nucleotide sequence encoding a NPT mutant or a non-naturally occurring NPT. In another embodiment, a kit comprises, in a container, a cDNA or genomic library or individual clones that contain nucleic acid sequence or nucleotide sequence encoding a NPT mutant or a non-naturally occurring NPT. In some embodiments, the NPT mutant nucleic acid sequence is one described in Section 7.2 or Section 8. In certain specific embodiments, the NPT mutant nucleic acid sequence is selected from the group consisting of SEQ ID NO:20, SEQ ID NO:32, SEQ ID NO:33, SEQ ID NO:34, SEQ ID NO:36 and SEQ ID NO:37. In some embodiments, a kit further comprises, in a container, neomycin, kanamycin or G418, or a derivative of any of the foregoing. In certain embodiments, a kit comprises, in a container, cells (e.g., host cells) in which a nucleic acid sequence comprising a nucleotide sequence encoding a NPT mutant or a non-naturally occurring NPT, or a vector (e.g., an expression vector) comprising a nucleic acid sequence or nucleotide sequence encoding a NPT mutant or a non-naturally occurring NPT may be introduced. In some embodiments, a kit further, in a container, comprises cells (e.g., host cells) in which a nucleic acid sequence comprising a nucleotide sequence encoding a NPT mutant or a non-naturally occurring NPT, or a vector (e.g., an expression vector) comprising a nucleic acid sequence or nucleotide sequence encoding a NPT mutant or a non-naturally occurring NPT has been introduced.

In certain embodiments, provided herein is a kit comprising, in a container, a vector comprising a nucleic acid sequence, wherein the nucleic acid sequence comprises a nucleotide sequence encoding a NPT mutant or a non-naturally occurring NPT. The vector may be a plasmid, phase, virus, cosmid, or a bacterial artificial chromosome. In some embodiments, provided herein is a kit comprising, in a container, a genomic sequence, a cDNA sequence, a genomic library, or an individual clone comprising a nucleic acid sequence, wherein the nucleic acid sequence comprises a nucleotide sequence encoding a NPT mutant or a non-naturally occurring NPT. In some embodiments, a kit further comprises, in a container, neomycin, kanamycin or G418, or a derivative of any of the foregoing.

In some embodiments, a kit comprises, in a container, synthetic DNA fragments or fragments not propagated in living cells that encode fragments of a NPT mutant or a non-naturally NPT described herein. Two or more complementary fragments of the NPT mutant or the non-naturally NPT can be in separate pieces in vectors, and the NPT mutant gene or the non-naturally NPT is reconstituted from the separate pieces when introduced into a host cell.

In some embodiments, provided herein is a kit comprising, in a container, a host cell described herein.

### 8. EXAMPLES

### 8.1 EXAMPLE 1: Identifying NPT Mutants with Reduced Activity

This example describes how NPT mutants were made and screened for reduced phosphotransferase activity.

### Construction of Plasmid Expression Vector

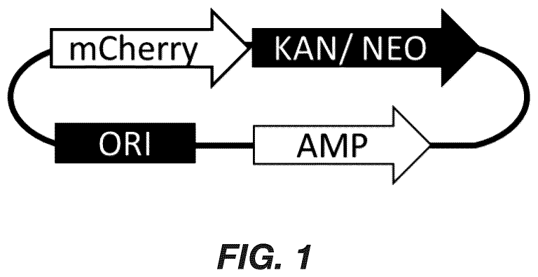

Plasmid vector P313 was constructed (FIG. 1, SEQ ID NO:2). It encodes an mCherry fluorescent protein expression cassette comprising an Human Elongation Factor alpha promoter and first intron (SEQ ID NO:3), the mCherry coding region (SEQ ID NO:4), and an SV40 polyadenylation signal (SEQ ID NO:5). P313 encodes the Neomycin phosphotransferase (NPT) protein derived from transposon Tn5 (aminoglycoside phosphotransferase3′-IIa) (SEQ ID NO:1; nucleotide sequence comprising SEQ ID NO:6) driven by the mouse Phosphoglycerate kinase promoter (SEQ ID NO: 7) for expression in mammalian cells and by the E. coli laczya promoter (SEQ ID NO: 8) for expression in bacteria. NPT transcription is terminated in mammalian cells by the Herpes Simplex Virus thymidine kinase polyadenylation signal (SEQ ID NO:9). The plasmid also encodes an ampicillin resistance gene (SEQ ID NO:10) and the pUC57 plasmid replication origin (SEQ ID NO:11).

Plasmids containing mutations in the NPT gene were created by replacing portions of the NPT open reading frame with DNA fragments generated by gene synthesis (Integrated DNA Technologies, Coralville IA). Plasmid P313 was digested with the appropriate pairs of restriction endonucleases with unique sites (including Bsp E1, Tth111 I, Rsr II, and Avr II) to create recipient vectors. Cloning mixtures contained 5 μl 2×HiFi cloning Mix, 50 ng synthetic DNA and 509 ng of the digested vector. Mixtures were incubated at 50° C. 15 min, and cooled to 4° C. 2 μl was transformed into Top10 Competent cells (Invitrogen) or Stellar competent cells (Clontech), plated onto LB-Carbenicillin plates, and incubated at 37° C. Single colonies were inoculated into 5 ml LB-Carbenicillin cultures and grown overnight in a shaking incubator at 37° C. DNAs were purified using the Qiagen spin miniprep kit (Qiagen). Plasmid sequences were verified by DNA sequencing (GENEWIZ, Plainfield, NJ). NPT activity was screened on plates containing kanamycin at concentrations of 25 μg/mL (KAN25) 50 μg/mL (KAN50), 75 μg/mL (KAN75), and 100 μg/mL (KAN100) as described below.

### Screening NPT Mutants in Bacteria

Overnight cultures grown in LB-Carbenicillin were serially diluted with PBS and plated onto LB-Carbenicillin, LB-KAN25, and LB-KAN100 plates and incubated for 24 hours. Colonies were counted, allowed to incubate for an additional 24 hours at 37° C. and were recounted. Plasmids where the colony numbers were significantly reduced but not absent on KAN100 plates relative to KAN25 and Carbenicillin plates were replated onto Carbenicillin, KAN25, KAN50, KAN75, and KAN100 and incubated and counted as above. The resulting colony numbers from forty-eight hour incubations are shown in Table 1.

TABLE 1
Colony Numbers Observed in Cells Expressing Mutant NPTs
Results after 48 hours; “n.d.” means not determined; the open reading frame for mutant
NPT nucleic acid sequences (“Neo ORF”) are identified by sequence identification numbers.

| | | | Neo | | | | | | | Host |
|---|---|---|---|---|---|---|---|---|---|---|
| Construct | Plasmid | Clone | ORF | Mutation(s) | Carb | KAN25 | KAN50 | KAN75 | KAN100 | Strain |
| A | P614 | P614-1 | SEQ ID | H188L | 183 | 214 | n.d. | n.d. | 201 | Top 10 |
| | | | NO: 12 | | | | | | | |
| B | P615 | P615-1 | SEQ ID | R211G | 116 | 115 | 133 | 131 | 94 | Top 10 |
| | | | NO: 13 | | | | | | | |
| C | P616 | P616-1 | SEQ ID | D261N | 153 | 74 | 61 | 64 | 0 | Top 10 |
| | | | NO: 14 | | | | | | | |
| D | P641 | P641- | SEQ ID | G205E | 132 | 0 | n.d. | n.d. | 0 | Top 10 |
| | | 16 | NO: 21 | | | | | | | |
| E | P642 | P642- | SEQ ID | D208G | 51 | 0 | n.d. | n.d. | 0 | Top 10 |
| | | 21 | NO: 22 | | | | | | | |
| F | P643 | P643- | SEQ ID | D216G | 100 | 116 | 126 | 117 | 106 | Top 10 |
| | | 26 | NO: 23 | | | | | | | |
| G | P679 | P679-1 | SEQ ID | G210A | 59 | 60 | n.d. | n.d. | 59 | Top 10 |
| | | | NO: 28 | | | | | | | |
| H | P680 | P680-2 | SEQ ID | Y218A | 133 | 190 | n.d. | n.d. | 156 | Top 10 |
| | | | NO: 29 | | | | | | | |
| I | P681 | P681-2 | SEQ ID | Y218F | 129 | 89 | n.d. | n.d. | 140 | Top 10 |
| | | | NO: 30 | | | | | | | |
| J | P682 | P682-1 | SEQ ID | V36M | 49 | 75 | 67 | 66 | 72 | Top 10 |
| | | | NO: 31 | | | | | | | |
| K | P623 | P623-4 | SEQ ID | H188L, | 210 | 38 | 43 | 32 | 13 | Top 10 |
| | | | NO: 15 | D261N | | | | | | |
| L | P624 | P624- | SEQ ID | R211G, | 126 | 0 | n.d. | n.d. | 0 | Top 10 |
| | | 33 | NO: 16 | D261N | | | | | | |
| M | P626 | P626-1 | SEQ ID | D190G1, | 251 | 0 | n.d. | n.d. | 0 | Top 10 |
| | | | NO: 17 | D261N | | | | | | |
| N | P629 | P629- | SEQ ID | D216G, | 74 | 78 | 0 | 0 | 0 | Top 10 |
| | | 11 | NO: 20 | D261N | | | | | | |
| O | P675 | P675-3 | SEQ ID | D227G, | 62 | 64 | n.d. | n.d. | 0 | Top 10 |
| | | | NO: 24 | D261N | | | | | | |
| P | P676 | P676-4 | SEQ ID | Y218D, | 240 | 0 | n.d. | n.d. | 0 | Top 10 |
| | | | NO: 25 | D261N | | | | | | |
| Q | P677 | P677- | SEQ ID | H188S, | 180 | 0 | n.d. | n.d. | 0 | Top 10 |
| | | 14 | NO: 26 | D261N | | | | | | |
| R | P678 | P678- | SEQ ID | E182D, | 320 | 7 | n.d. | n.d | 0 | Top 10 |
| | | 11 | NO: 27 | D261N | | | | | | |
| S | P683 | P683-6 | SEQ ID | V36M, | 76 | 41 | 0 | 0 | 0 | Stellar |
| | | | NO: 32 | G210A | | | | | | |
| T | P684 | P684-1 | SEQ ID | V36M, | 96 | 43 | 0 | 0 | 0 | Stellar |
| | | | NO: 33 | Y218S | | | | | | |
| U | P685 | P685-1 | SEQ ID | V36M, | 170 | 168 | 206 | 170 | 1 | Stellar |
| | | | NO: 34 | Y218F | | | | | | |
| V | P686 | P686-2 | SEQ ID | V36M, | 142 | 0 | 0 | 0 | 0 | Stellar |
| | | | NO: 35 | H188S | | | | | | |
| W | P687 | P687-2 | SEQ ID | V36M, | 47 | 44 | 23 | 72 | 0 | Stellar |
| | | | NO: 36 | E182D | | | | | | |
| X | P688 | P688-9 | SEQ ID | V36M, | 72 | 36 | 0 | 0 | 0 | Stellar |
| | | | NO: 37 | D216G | | | | | | |

### Results

Two of the single site point mutants resulted in a complete loss of activity in this assay (G205E and D208G). Only two of the remaining 8 mutants showed decreased activity in this assay. Mutant R211G also grew much more slowly on KAN100 plates even though the total colony numbers were similar to growth on lower KAN concentrations. D261N was incapable of growing on KAN100 plates and only produced about half as many colonies on the other KAN plates. Four of the mutants that showed full activity in our assay (G210A, Y218S, Y218F, and V36M) had been previously reported to confer decreased resistance to kanamycin (Blazquez (1991) Mol. Microbiol. 5:1511-1518; Kocabiyik (1992) Biochem. Biophys. Res. Commun. 185: 925-931; Kocabiyik (1992) FEMS Microbiol Lett 93: 199-202). It is possible that these NPT mutants are expressed at a higher level than in previous studies through use of a high-copy plasmid and/or a stronger bacterial promoter.

Double mutants with D261N were constructed to identify those with even less activity. Four D261N double mutants were completely deficient, and one (E182D; D261N) was extremely deficient (2 percent of the colonies on KAN25 relative to Carbenicillin plates and no growth at other kanamycin concentrations). Two clones only produced colonies on KAN25 plates but colony numbers were similar to those on carbenicillin plates (i.e. clone N, (D216G; D261N) and clone O (D227G; D261N). One mutation appeared to partially complement the D261N mutation, allowing growth on KAN100 plates, albeit at a reduced efficiency relative to growth on carbenicillin plates (clone K (H188L, D261N).

Four clones (S, T, U, and V) combined two mutations that independently had full activity above but that had been previously reported to have reduced activity (Blazquez (1991) Mol. Microbiol. 5:1511-1518; Kocabiyik (1992) Biochem. Biophys. Res. Commun. 185: 925-931; Kocabiyik (1992) FEMS Microbiol Lett 93: 199-202). Two additional clones combined V36M with mutations that were not tested above. Mutation H188S reportedly reduced resistance to kanamycin (Blazquez (1991) Mol. Microbiol. 5:1511-1518) while mutation E182D was reported to reduce resistance to G418 but not kanamycin (Yenofsky (1990) Proc. Natl. Acad. Sci. USA 87:3435-3439). The clone containing the V36M; H188S mutations was completely deficient. Three clones only retained the ability to grow on KAN25 plates, while the two remaining clones only displayed growth deficits on KAN100 plates (1 and 0 colonies for clones U (V36M; Y218F) and W (V36M and E182D) respectively). These results demonstrate that combining certain mutations that individually have weak or no effect on NPT activity surprisingly produces double mutant NPTs with activities suitable for numerous applications.

### 8.2 Example 2: Mutant NPT Proteins as Selection Markers in HEK293 Cells

To demonstrate that plasmids containing attenuated NPT gene cassettes could still confer resistance to G418 in human cells, several of the plasmids constructed above were transfected into HEK293 cells and were subjected to a colony formation assay. DNAs were purified from 200 ml LB-cultures containing carbenicillin using Qiagen's HiSpeed maxiprep kit following the manufacturer's instructions.

For transfection, 2E7 HEK293 cells were plated into eight T-75 flasks in 40 ml growth media (DMEM+10% FBS+1×PenStrep) and incubated at 37° C. Transfections were assembled in 15 ml Corning tubes and contained 22 μg DNA+3 mls of OptiMEM at 37° C.+66 μl Fugene-6 transfection reagent. The transfection mix was vortexed briefly and incubated in a 37° C. CO₂ incubator for 15 minutes. Growth medium (2 ml) was added and the entire mix was added to a flask of HEK293 cells plated earlier. Flasks were incubated at 37° C. After 48 hours, all the flasks had cells with bright red fluorescence. Flasks were washed with 10 mls PBS, and 1 ml TryPLE and incubated at 37° C. for 5 minutes. Cells were washed from flasks with 10 ml growth medium and were subsequently replated into T150 flasks in 25 ml medium and incubated for 48 hours at 37° C. Cells were then recovered from growth surfaces as before and cell density was determined using duplicate readings using the Countess cell counter. Serial dilutions were plated into duplicate 150 mm plates with the Nuclon Delta Surface in 50 mls of selective growth medium (DMEM+10% FBS+1×PenStrep+500 μg/ml Geneticin). Plates were incubated for 18 days and plates with transfection of plasmids P313, C and S were stained and photographed. Plates from the other transfections were incubated for another 13 days before being stained and photographed. For staining, media was gently removed by pipetting. Cells were covered with 10 mls staining solution (0.4% Methylene Blue in 50% Methanol) and incubated for 10 minutes at room temperature. Staining solution was removed by pipetting and cells were washed with 5 ml 100% methanol and air dried. Plates were photographed using Bio-Rad Imaging station.

### Results

The results from the colony formation assay are presented in Table 2. Four of the mutant constructs produced G418 resistant colonies at frequencies ranging from 5.5% to 0.004% of the frequency measured for construct P313 with the wild type NPT gene.

In this assay, colony formation frequency is an indirect measure of NPT protein activity. Cells that express more of the mutant NPT as compared to other cells in the population of transfected cells, whether due to greater multi-copy integration of the expression cassette and/or due to more favorable genomic location of integration of the cassette, are able to survive to form colonies when grown in the presence of G418. The results of this example demonstrate that use of a NPT mutant with reduced activity as a selection marker can be used to reduce time and effort of having to screen multiple colonies for stably integrated, high transgene expressing cells.

Three of the mutant constructs with the most attenuated phenotypes in bacteria failed to produce G418 resistant colonies from the 1E7 cells plated. While it is possible that these mutant proteins are completely inactive in mammalian cells, it is also possible that cells expressing sufficiently high levels would survive selection. Such markers may be useful in combination with methods that are more efficient at generating high copy number integrations such as retroviral infection or transposition.

TABLE 2
Colony Formation Frequency of HEK Cells Transfected with Mutant NPT Expression Cassette

| | | | | Average | | |
|---|---|---|---|---|---|---|
| | | Cells Plated | Number of | Colonies per | Cells Per | Colony Formation |
| Contruct | Mutations | Per Dish | Plates | Plate | Colony | Frequency |
| P313 | none | 5.0E+03 | 2 | 76 ± 13 | 66 | 100% |
| C | D261N | 1.00E+05 | 2 | 84 ± 6 | 1,190 | 5.5% |
| S | V36M G210A | 1.50E+05 | 2 | 23 ± 3 | 6,522 | 1.0% |
| W | V36M E182D | 2.50E+06 | 4 | 7.5 ± 3.5 | 333,333 | 0.02% |
| U | V36M Y218F | 2.50E+06 | 4 | 1.5 ± 0.6 | 1,666,667 | 0.004% |
| N | D216G; D261N | 2.50E+06 | 4 | 0 | N/A | |
| T | V36M Y218S | 2.50E+06 | 4 | 0 | N/A | |
| X | V36M D216G | 2.50E+06 | 4 | 0 | N/A | |

### 8.3 Example 3: Introduction of Transgene by Transposase

This example demonstrates the integration of a mCherry and NPT expression cassette into human cells using transposase activity. NPT mutants as described herein are used in this example.

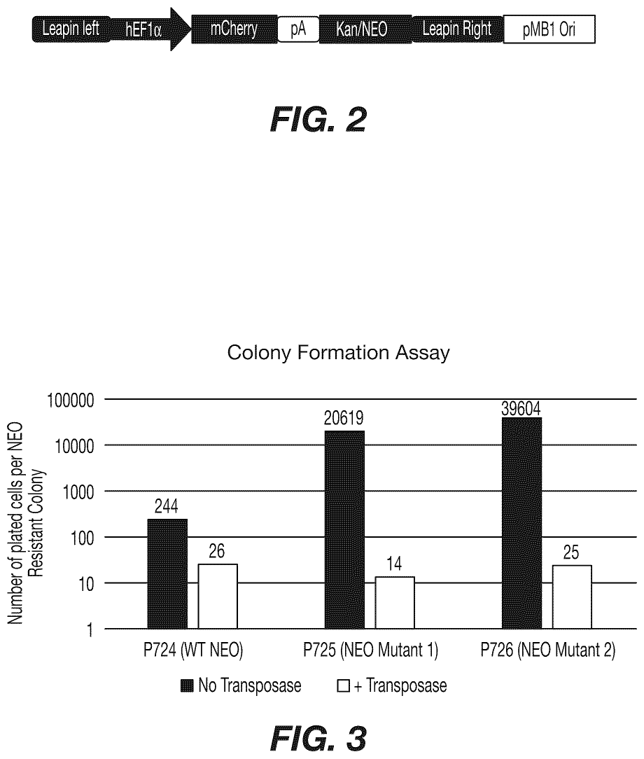

Three different constructs with the configuration depicted in FIG. 2 were produced. The constructs differed from each other in that they contained a nucleic acid sequence encoding either wild-type neomycin phosphotransferase, mutant 1 (P725) neomycin phosphotransferase (V36M; G210A), or mutant 2 (P726) neomycin phosphotransferase (E182D; D261N). The constructs were electroporated into human VPC cells (HEK293 variant) with or without Leap-In Transposase RNA (ATUM Design, Newark, CA). Cells were plated onto 150 mm plates, and cultured for 2 weeks under neomycin selection. Cells were then stained and measured for colony formation. 8-12 colonies from different plates that were unstained were selected and mCherry copy number was measured relative to the endogenous glutamine synthetase gene droplet digital PCR (ddPCR).

### Results

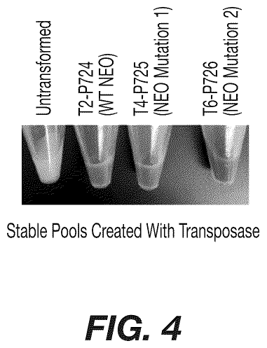

Results from a colony formation assay are shown in FIG. 3, which shows that NPT mutants dramatically decreased the efficiency of colony formation by random integration of the expression construct but not by transposition. FIG. 4 is a picture of a stable pools of cells created with transposase where the color produced by mCherry expression is clearly evident in normal white light illumination when compared to untransformed cells that lack color.

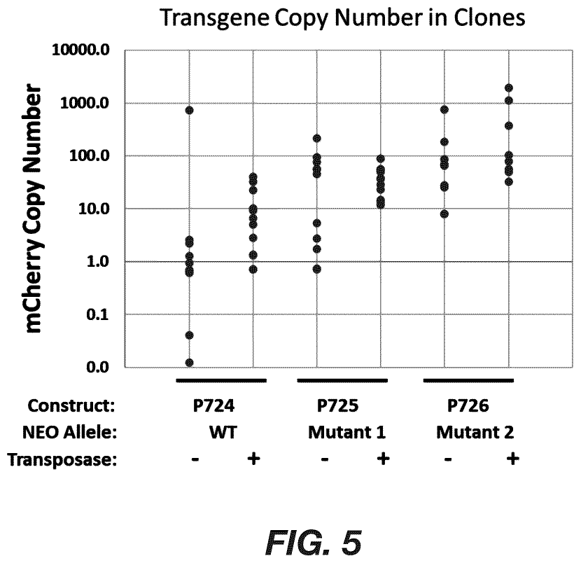

Results from a measurement of mCherry copy number in selected clones are shown in FIG. 5. The results demonstrate that NPT mutant-containing cells have consistently higher average copy numbers of the linked mCherry transgene relative to those with wild-type NPT. Most of the clones generated by random integration of the construct with the wild-type NPT gene had little if any fluorescence, while most of the clones derived by random integration of the two mutant NPT genes were fluorescent. This can be interpreted to mean that the mutant NPT genes must be expressed at a higher level than the wild-type NPT gene for survival during G418 selection, whether through increased copy numbers or through integration in a favorable genomic location, and this results in increased expression of the mCherry transgene.

The enzymatic integration of transgenes into host chromosomes by transposition was much more efficient than random integration and resulted in higher average copy numbers even with using the wild-type NPT gene. The mutant NPT genes also increase the copy numbers relative to use of the wild-type NPT gene, which would provide an advantage in cases where gene delivery or transposition is inefficient such as in the case of large constructs.

### 9. EMBODIMENTS

This invention provides the following non-limiting embodiments.

In one set of embodiments, provided are:
A1. A non-naturally occurring neomycin phosphotransferase (NPT) with neomycin phosphotransferase activity, wherein the non-naturally occurring NPT comprises an amino acid sequence of a wild-type neomycin phosphotransferase with:
   (a) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
(b) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
(c) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
(d) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
(e) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
(f) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
A2. A non-naturally occurring neomycin phosphotransferase (NPT) with neomycin phosphotransferase activity, wherein the non-naturally occurring NPT comprises the amino acid sequence of SEQ ID NO:1 with:
(a) amino acid substitutions at positions 36 and 210 of SEQ ID NO:1, wherein the amino acid substitution at position 36 of SEQ ID NO:1 is a substitution to methionine and the amino acid substitution at position 210 of SEQ ID NO:1 is a substitution to alanine;
   (b) amino acid substitutions at positions 36 and 182 of SEQ ID NO:1, wherein the amino acid substitution at position 36 of SEQ ID NO:1 is a substitution to methionine and the amino acid substitution at position 182 of SEQ ID NO:1 is a substitution to aspartic acid;
   (c) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to phenylalanine;
   (d) amino acid substitutions at positions 216 and 261 of SEQ ID NO:1, wherein the amino acid substitution position 216 of SEQ ID NO:1 is a substitution to glycine and the amino acid substitution at position 261 of SEQ ID NO:1 is a substitution to asparagine;
   (e) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to serine; or
   (f) amino acid substitutions at positions 36 and 216 of SEQ ID NO:1, wherein the amino acid substitution at position 36 of SEQ ID NO:1 is a substitution to methionine and the amino acid substitution at position 216 of SEQ ID NO:1 is a substitution to glycine.
A3. The NPT of embodiment A1, wherein the non-naturally occurring NPT has attenuated neomycin phosphotransferase activity as a selectivity marker as compared to wild-type NPT.
A4. The NPT of embodiment A1 or A3, wherein the wild-type NPT comprises an amino acid sequence that is at least 80%, at least 90%, or at least 98% identical to SEQ ID NO:1.
A5. The NPT of embodiment A1 or A3, wherein the wild-type NPT comprises an amino acid sequence that is at least 60%, at least 65%, at least 70% or at least 75% identical to SEQ ID NO:1.
A6. The NPT of embodiment A2, wherein the non-naturally occurring NPT has attenuated neomycin phosphotransferase activity as a selectivity marker as compared to wild-type NPT comprising the amino acid sequence of SEQ ID NO:1.
A7. The NPT of embodiment A1, A3, A4 or A5, wherein bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT.
A8. The NPT of embodiment A7, wherein the bacterial cells are E. coli.
A9. The NPT of embodiment A1, A3, A4 or A5, wherein mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT.
A10. The NPT of embodiment A9, wherein the mammalian cells are HEK293 cells, CHO cells, PER.C6 cells, murine NS0 cells, fibrosarcoma HT-1080 cells, murine Sp2/0 cells, BHK cells, or murine C127 cells.
A11. The NPT of embodiment A1, A3, A4 or A5, wherein G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT.
A12. The NPT of embodiment A2, wherein bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT comprising the amino acid sequence of SEQ ID NO:1.
A13. The NPT of embodiment A12, wherein the bacterial cells are E. coli.
A14. The NPT of embodiment A2, wherein mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT, wherein the wild-type NPT comprises the amino acid sequence of SEQ ID NO: 1.
A15. The NPT of embodiment A14, wherein the mammalian cells are HEK293 cells, CHO cells, PER.C6 cells, murine NS0 cells, fibrosarcoma HT-1080 cells, murine Sp2/0 cells, BHK cells, or murine C127 cells.
A16. The NPT of embodiment A2, wherein G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT, wherein the wild-type NPT comprises the amino acid sequence of SEQ ID NO:1.
A17. The NPT of any one of embodiments A1, A3, A4, A5, or A7 to A11, wherein the non-naturally occurring NPT comprises at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.
A18. The NPT of any one of embodiments A1, A3, A4, A5, or A7 to A11, wherein the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.
A19. The NPT of any one of embodiments A1, A3, A4, A5, or A7 to A11, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine.
A20. The NPT of any one of embodiments A1, A3, A4, A5, or A7 to A11, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.
A21. The NPT of any one of embodiments A1, A3, A4, A5, or A7 to A11, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine.
A22. The NPT of any one of embodiments A1, A3, A4, A5, or A7 to A11, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
A23. The NPT of any one of embodiments A2 or A12 to A16, wherein the non-naturally occurring NPT comprises the amino acid sequence of SEQ ID NO:38 (V36M, G210A).
A24. The NPT of any one of embodiments A2 or A12 to A16, wherein the non-naturally occurring NPT comprises the amino acid sequence of SEQ ID NO:39 (V36M, E182D).
A25. The NPT of any one of embodiments A2 or A12 to A16, wherein the non-naturally occurring NPT comprises the amino acid sequence of SEQ ID NO:40 (V36M, Y218F).
A26. The NPT of any one of embodiments A2 or A12 to A16, wherein the non-naturally occurring NPT comprises the amino acid sequence of SEQ ID NO:41 (D216G, D261N).
A27. The NPT of any one of embodiments A2 or A12 to A16, wherein the non-naturally occurring NPT comprises the amino acid sequence of SEQ ID NO:42 (V36M, Y218S).
A28. The NPT of any one of embodiments A2 or A12 to A16, wherein the non-naturally occurring NPT comprises the amino acid sequence of SEQ ID NO:43 (V36M, D216G).
A29. A nucleic acid sequence comprising a first nucleotide sequence encoding the non-naturally occurring NPT of any one of embodiments A1 to A28.
A30. The nucleic acid sequence of embodiment of A29, wherein the nucleic acid sequence further comprises a second nucleotide sequence encoding a second protein or a non-coding RNA.
A31. The nucleic acid sequence of embodiment A30, wherein the second nucleotide sequence encodes a second protein and wherein the second protein is a therapeutic protein.
A32. The nucleic acid sequence of any one of embodiments A29 to A31, wherein the first nucleotide sequence comprises the nucleotide sequence of SEQ ID NO:20, SEQ ID NO:32, SEQ ID NO:33, SEQ ID NO:34, SEQ ID NO: 36, or SEQ ID NO:37.
A33. A vector comprising the nucleic acid sequence of any one of embodiments A29 to A32.
A34. An in vitro or ex vivo host cell comprising the non-naturally occurring NPT of any one of embodiments A1 to A28.
A35. An in vitro or ex vivo host cell comprising the nucleic acid sequence of any one of embodiments A29 to A32.
A36. The cell of embodiment A35, wherein the nucleic acid sequence is stably integrated into the genome of the host cell.
A37. An in vitro or ex vivo host cell comprising the vector of embodiment A33.
A38. The host cell of any one of embodiments A34 to A37, wherein the host cell is a bacterium, yeast cell, mammalian cell, or plant cell.
A39. The host cell of any one of embodiments A34 to A37, wherein the host cell is from a human cell line.

In a second set of embodiments, provided are:
B1. An in vitro or ex vivo host cell expressing a non-naturally occurring NPT, wherein the non-naturally occurring NPT is attenuated relative to a wild-type neomycin phosphotransferase, and wherein the non-naturally occurring NPT comprises an amino acid sequence of the wild-type neomycin phosphotransferase with:
   (a) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
   (b) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
   (c) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
   (d) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
   (e) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
   (f) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
B2. An in vitro or ex vivo host cell expressing a non-naturally occurring NPT with neomycin phosphotransferase activity, wherein the non-naturally occurring NPT is attenuated relative to a wild-type neomycin phosphotransferase, and wherein the non-naturally occurring NPT comprises the amino acid sequence of SEQ ID NO:1 with:
   (a) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine;
   (b) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid;
   (c) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine;
   (d) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine;
   (e) amino acid substitutions at amino acid residues 36 and 218 of SEQ ID NO:1, wherein the amino acid substitution at amino acid residue 36 of SEQ ID NO:1 is a substitution to methionine and the amino acid substitution amino acid residue 218 of SEQ ID NO:1 is a substitution to serine; or
   (f) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine.
B3. The cell of embodiment B1, wherein the wild-type NPT comprises an amino acid sequence that is at least 80%, at least 90%, or at least 98% identical to SEQ ID NO:1. B4. The cell of embodiment B1, wherein the wild-type NPT comprises an amino acid sequence that is at least 60%, at least 65%, at least 70%, or at least 75% identical to SEQ ID NO:1.
B5. The cell of embodiment B2, wherein the non-naturally occurring NPT has attenuated neomycin phosphotransferase activity as a selectivity marker as compared to wild-type NPT comprising the amino acid sequence of SEQ ID NO:1.
B6. The cell of embodiment B1, B3, or B4, wherein bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT.
B7. The cell of embodiment B6, wherein the bacterial cells are E. coli.
B8. The cell of embodiment B1, B3, or B4, wherein mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT.
B9. The cell of embodiment B8, wherein the mammalian cells are HEK293 cells, CHO cells, PER.C6 cells, murine NS0 cells, fibrosarcoma HT-1080 cells, murine Sp2/0 cells, BHK cells, or murine C127 cells.
B10. The cell of embodiment B1, B3, or B4, wherein G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT.
B11. The cell of embodiment B2 or B5, bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT comprising the amino acid sequence of SEQ ID NO:1.
B12. The cell of embodiment B11, wherein the bacterial cells are E. coli.
B13. The cell of embodiment B2 or B5, wherein mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT, wherein the wild-type NPT comprises the amino acid sequence of SEQ ID NO: 1.
B14. The cell of embodiment B13, wherein the mammalian cells are HEK293 cells, CHO cells, PER.C6 cells, murine NS0 cells, fibrosarcoma HT-1080 cells, murine Sp2/0 cells, BHK cells, or murine C127 cells.
B15. The cell of embodiment B2 or B5, wherein G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT, wherein the wild-type NPT comprises the amino acid sequence of SEQ ID NO:1.
B16. The cell of any one of embodiments B1, B3, B4, or B6 to B10, wherein the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.
B17. The cell of any one of embodiments B1, B3, B4, or B6 to B10, wherein the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.
B18. The cell of any one of embodiments B1, B3, B4, or B6 to B10, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to phenylalanine.
B19. The cell of any one of embodiments B1, B3, B4, or B6 to B10, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.
B20. The cell of any one of embodiments B1, B3, B4, or B6 to B10, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine.
B21. The cell of any one of embodiments B1, B3, B4, or B6 to B10, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
B22. The cell of any one of embodiments B2, B5 or B11 to B15, wherein the non-naturally occurring NPT comprises the amino acid sequence of SEQ ID NO:38 (V36M, G210A).
B23. The cell of any one of embodiments B2, B5 or B11 to B15, wherein the non-naturally occurring NPT comprises the amino acid sequence of SEQ ID NO:39 (V36M, E182D).
B24. The cell of any one of embodiments B2, B5 or B11 to B15, wherein the non-naturally occurring NPT comprises the amino acid sequence of SEQ ID NO:40 (V36M, Y218F).
B25. The cell of any one of embodiments B2, B5 or B11 to B15, wherein the non-naturally occurring NPT comprises the amino acid sequence of SEQ ID NO:41 (D216G, D261N).
B26. The cell of any one of embodiments B2, B5 or B11 to B15, wherein the non-naturally occurring NPT comprises the amino acid sequence of SEQ ID NO:42 (V36M, Y218S).
B27. The cell of any one of embodiments B2, B5 or B11 to B15, wherein the non-naturally occurring NPT comprises the amino acid sequence of SEQ ID NO:43 (V36M, D216G).
B28. The cell of any one of embodiments B1 to B27, wherein the cell further comprises a second nucleic acid sequence encoding a second protein or a non-coding RNA.
B29. The cell of embodiment B28, wherein the second nucleic acid sequence encodes a second protein and wherein the second protein is a therapeutic protein.
B30. The cell of embodiment B28, wherein the second nucleic acid sequence encodes a non-coding RNA, and wherein the non-coding RNA is shRNA, miRNA, antisense RNA, guide RNA for Crispr nucleases, catalytic RNA, ribosomal RNA, or tRNA.
B31. The cell of any one of embodiments B1 to B30, wherein the host cell is a bacterium, yeast cell, mammalian cell, or plant cell.

In a third set of embodiments, provided are:
C1. A method for selecting cells with high copy numbers of a transgene and/or high expression levels of a transgene from a population of host cells in which the transgene was introduced, the method comprising:
a) introducing into a population of host cells a nucleic acid sequence comprising:
   (i) a first nucleotide sequence encoding a non-naturally occurring neomycin phosphotransferase (NPT) with neomycin phosphotransferase activity; and
   (ii) a second nucleotide sequence comprising the transgene,
   wherein the non-naturally occurring NPT comprises an amino acid sequence of a wild-type neomycin phosphotransferase with:
      (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
      (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
      (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
      (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
      (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and
   b) selecting cells that grow in the presence of a neomycin phosphotransferase substrate from the population of host cells in which the nucleic acid sequence was introduced.
C2. A method for selecting cells with high copy numbers of a transgene and/or high expression levels of a transgene from a population of host cells in which the transgene was introduced, the method comprising:
   a) introducing into a population of host cells a nucleic acid sequence comprising:
      (i) a first nucleotide sequence encoding a non-naturally occurring neomycin phosphotransferase (NPT) with neomycin phosphotransferase activity; and
      (ii) a second nucleotide sequence comprising the transgene,
   wherein the non-naturally occurring NPT comprises the amino acid sequence of SEQ ID NO:1 with:
      (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine;
      (2) amino acid substitutions at amino acid residues 36 and 182 of SEQ ID NO:1, wherein the amino acid substitution at the amino acid residue 36 of SEQ ID NO:1 is a substitution to methionine and the amino acid substitution at amino acid residue 182 of SEQ ID NO:1 is a substitution to aspartic acid;
      (3) amino acid substitutions at amino acid residues 36 and 218 of SEQ ID NO:1, wherein the amino acid substitution at the amino acid residue 36 of SEQ ID NO:1 is a substitution to methionine and the amino acid substitution at amino acid residue 218 of SEQ ID NO:1 is a substitution to phenylalanine;
      (4) amino acid substitutions at amino acid residues 216 and 261 of SEQ ID NO:1, wherein the amino acid substitution at the amino acid residue 216 of SEQ ID NO:1 is a substitution to glycine and the amino acid substitution at amino acid residue 261 of SEQ ID NO:1 is a substitution to asparagine;
      (5) amino acid substitutions at amino acid residues 36 and 218 of SEQ ID NO:1, wherein the amino acid substitution at the amino acid residue 36 of SEQ ID NO:1 is a substitution to methionine and the amino acid substitution amino acid residue 218 of SEQ ID NO:1 is a substitution to serine; or
      (6) amino acid substitutions at amino acid residues 36 and 216 of SEQ ID NO:1, wherein the amino acid substitution at the amino acid residue 36 of SEQ ID NO:1 is a substitution to methionine and the amino acid substitution at amino acid residue 216 of SEQ ID NO:1 is a substitution to glycine; and
   b) selecting cells that grow in the presence of a neomycin phosphotransferase substrate from the population of host cells in which the nucleic acid sequence was introduced.
C3. The method of embodiment C1, wherein the non-naturally occurring NPT has attenuated neomycin phosphotransferase activity as a selectable marker as compared to wild-type NPT.
C4. The method of embodiment C1 or C3, wherein the wild-type NPT comprises an amino acid sequence that is at least 80%, at least 90%, or at least 98% identical to SEQ ID NO:1.
C5. The method of embodiment C1, wherein the wild-type NPT comprises an amino acid sequence that is at least 60%, at least 65%, at least 70%, or at least 75% identical to SEQ ID NO:1.
C6. The method of embodiment C2, wherein the non-naturally occurring NPT has attenuated neomycin phosphotransferase activity as a selectable marker as compared to wild-type NPT comprising the amino acid sequence of SEQ ID NO:1.
C7. The method of embodiment C1, C3, C4 or C5, wherein bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT.
C8. The method of embodiment C7, wherein the bacterial cells are E. coli.
C9. The method of embodiment C1, C3, C4 or C5, wherein mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT.
C10. The method of embodiment C9, wherein the mammalian cells are HEK293 cells, CHO cells, PER.C6 cells, murine NS0 cells, fibrosarcoma HT-1080 cells, murine Sp2/0 cells, BHK cells, or murine C127 cells.
C11. The method of embodiment C1, C3, C4 or C5, wherein G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT.
C12. The method of embodiment C2 or C6, wherein bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT comprising the amino acid sequence of SEQ ID NO:1.
C13. The method of embodiment C12, wherein the bacterial cells are E. coli.
C14. The method of embodiment C2 or C6, wherein mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT, wherein the wild-type NPT comprises the amino acid sequence of SEQ ID NO:1.
C15. The method of embodiment C14, wherein the mammalian cells are HEK293 cells, CHO cells, PER.C6 cells, murine NS0 cells, fibrosarcoma HT-1080 cells, murine Sp2/0 cells, BHK cells, or murine C127 cells.
C16. The method of embodiment C2 or C6, wherein G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT, wherein the wild-type NPT comprises the amino acid sequence of SEQ ID NO:1.
C17. The method of any one of embodiments C1, C3, C4, C5, or C7 to C11, wherein the non-naturally occurring NPT comprises the amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.
C18. The method of any one of embodiments C1, C3, C4, C5, or C7 to C11, wherein the NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.
C19. The method of any one of embodiments C1, C3, C4, C5, or C7 to C11, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine.
C20. The method of any one of embodiments C1, C3, C4, C5, or C7 to C11, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.
C21. The method of any one of embodiments C1, C3, C4, C5, or C7 to C11, wherein the non-naturally occurring NPT comprises amino acid substitutions at amino acid residues corresponding to amino acid residues 36 and 218 of SEQ ID NO:1, wherein the amino acid substitution at the amino acid residue corresponding to the amino acid residue 36 of SEQ ID NO:1 is a substitution to methionine and the amino acid substitution at amino acid residue corresponding to amino acid residue 218 of SEQ ID NO:1 is a substitution to serine.
C22. The method of any one of embodiments C1, C3, C4, C5, or C7 to C11, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
C23. The method of embodiment C2 or C6, wherein the non-naturally occurring NPT comprises the amino acid sequence of SEQ ID NO:38 (V36M, G210A).
C24. The method of embodiment C2 or C6, wherein the non-naturally occurring NPT comprises the amino acid sequence of SEQ ID NO:39 (V36M, E182D).
C25. The method of embodiment C2 or C6, wherein the non-naturally occurring NPT comprises the amino acid sequence of SEQ ID NO:40 (V36M, Y218F).
C26. The method of embodiment C2 or C6, wherein the non-naturally occurring NPT comprises the amino acid sequence of SEQ ID NO:41 (D216G, D261N).
C27. The method of embodiment C2 or C6, wherein the non-naturally occurring NPT comprises the amino acid sequence of SEQ ID NO:42 (V36M, Y218S).
C28. The method of embodiment C2 or C6, wherein the non-naturally occurring NPT comprises the amino acid sequence of SEQ ID NO:43 (V36M, D216G).
C29. The method of any one of embodiments C1 to C28, wherein:
   (a) the selected cells comprise a 2 to 1000 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene; and/or
   (b) the selected cells achieve a 10 to 1000 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene.
C30. The method of any one of embodiments C1 to C29, wherein the host cells are bacterial, yeast, mammalian or plant cells.
C31. The method of any one of embodiments C1 to C29, wherein the host cells are human cells.
C32. The method of any one of embodiments C1 to C31, wherein the nucleic acid sequence is stably integrated into the genome of the selected cell.
C33. The method of any one of embodiments C1 to C32, wherein the selected cells have a high copy number of the transgene.
C34. The method of any one of embodiments C1 to C33, wherein the selected cells have high level of expression of the transgene.
C35. The method of any one of embodiments C1 to C34, wherein the selected cells have integrated 5 to 100 copies of the transgene into their genomic DNA.
C36. The method of any one of embodiments C1 to C35, wherein the selected cells have integrated 1 to 5 copies of the transgene into their genomic DNA.
C37. The method of any one of embodiments C1 to C36, wherein the transgene comprises a viral gene.
C38. The method of any one of embodiments C1 to C36, wherein the transgene comprises a human growth factor gene.
C39. The method of any one of embodiments C1 to C38, wherein the neomycin phosphotransferase substrate is neomycin, kanamycin or G418.
C40. A method of using a plasmid or transposon comprising a nucleic acid sequence encoding a non-naturally occurring NPT with attenuated neomycin phosphotransferase activity as compared to wild-type NPT as a selectable marker, the method comprising:
a) introducing into a host cell the plasmid or transposon comprising the nucleic acid sequence encoding the non-naturally occurring NPT, wherein the non-naturally occurring NPT comprises an amino acid sequence of a wild-type neomycin phosphotransferase with:
   (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
   (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
   (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
   (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
   (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
   (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and
b) growing the cell in the presence of a neomycin phosphotransferase substrate.
C41. A method of using a plasmid or transposon comprising a nucleic acid sequence encoding a non-naturally occurring NPT with attenuated neomycin phosphotransferase activity as compared to wild-type NPT as a selectable marker, the method comprising:
a) introducing into a host cell the plasmid or transposon comprising the nucleic acid sequence encoding the non-naturally occurring NPT, wherein the non-naturally occurring NPT comprises the amino acid sequence of SEQ ID NO:1 with:
   (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine;
   (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid;
   (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine;
   (4) amino acid substitutions at amino acid residues corresponding to amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine;
   (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or
   (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and
b) growing the cell in the presence of a neomycin phosphotransferase substrate.
C42. The method of embodiment C40, wherein the wild-type NPT comprises an amino acid sequence that is at least 80%, at least 90%, or least 98% identical to SEQ ID NO:1.
C43. The method of embodiment C40, wherein the wild-type NPT comprises an amino acid sequence that is at least 60%, at least 65%, least 70%, or at least 75% identical to SEQ ID NO:1.
C44. The method of embodiment C40, C42 or C43, wherein the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.
C45. The method of embodiment C40, C42 or C43, wherein the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.
C46. The method of embodiment C40, C42 or C43, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine.
C47. The method of embodiment C40, C42 or C43, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.
C48. The method of embodiment C40, C42 or C43, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine.
C49. The method of embodiment C40, C42 or C43, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
C50. The method of embodiment C41, wherein the non-naturally occurring NPT comprises the amino acid sequence of SEQ ID NO:38 (V36M, G210A).
C51. The method of embodiment C41, wherein the non-naturally occurring NPT comprises the amino acid sequence of SEQ ID NO:39 (V36M, E182D).
C52. The method of embodiment C41, wherein the non-naturally occurring NPT comprises the amino acid sequence of SEQ ID NO:40 (V36M, Y218F).
C53. The method of embodiment C41, wherein the non-naturally occurring NPT comprises the amino acid sequence of SEQ ID NO:41 (D216G, D261N).
C54. The method of embodiment C41, wherein the non-naturally occurring NPT comprises the amino acid sequence of SEQ ID NO:42 (V36M, Y218S).
C55. The method of embodiment C41, wherein the non-naturally occurring NPT comprises the amino acid sequence of SEQ ID NO:43 (V36M, D216G).
C56. The method of any one of embodiments C40 to C55, wherein the host cell is a bacterial, yeast, mammalian or plant cell.
C57. The method of any one of embodiments C40 to C55, wherein the host cell is a human cell.
C58. The method of any one of embodiments C40 to C55, wherein the plasmid or transposon further comprises a second nucleotide sequence encoding a protein or a non-coding RNA.
C59. The method of embodiment C58, wherein the protein is a viral protein.
C60. The method of embodiment C58, wherein the protein is a therapeutic protein.
C61. The method of any one of embodiments C40 to C60, wherein the neomycin phosphotransferase substrate is neomycin, kanamycin or G418.
C62. The method of any one of embodiments C1 to C39, wherein the transgene encodes a protein or a non-coding RNA.
C63. The method of embodiment C62, wherein the transgene encodes a non-coding RNA selected from the group consisting of antisense RNA, miRNA, shRNA, long non-coding RNA, catalytic RNA, ribosomal RNA, tRNA, or a guide RNA for a CRISPR nuclease.
C64. The method of embodiment C62, wherein the transgene encodes a protein and the protein is a therapeutic protein or antigen.

In a fourth set of embodiments, provided are:
D1. A method of making host cells comprising a second nucleotide sequence comprising:
   a) introducing into a population of host cells a first nucleic acid sequence comprising (i) a first nucleotide sequence encoding the non-naturally occurring NPT, and (ii) a second nucleotide sequence comprising a transgene encoding a second protein or a non-coding RNA, wherein the non-naturally occurring NPT comprises an amino acid sequence of a wild-type neomycin phosphotransferase with:
      (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
      (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
      (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
      (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
      (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine;
   b) growing the population of host cells in the presence of a neomycin phosphotransferase substrate to produce colonies; and
   c) selecting a colony of cells that grows in the presence of the neomycin phosphotransferase substrate.
D2. A method of making host cells comprising a second nucleotide sequence comprising:
   a) co-introducing into a population of host cells (i) a first nucleic acid sequence comprising a first nucleotide sequence encoding the non-naturally occurring NPT, and (ii) a second nucleic acid sequence comprising a transgene encoding a second protein or a non-coding RNA, wherein the non-naturally occurring NPT comprises the amino acid sequence of SEQ ID NO:1 with:
      (1) amino acid substitutions at positions 36 and 210 of SEQ ID NO:1, wherein the amino acid substitution at position 36 of SEQ ID NO:1 is a substitution to methionine and the amino acid substitution at position 210 of SEQ ID NO:1 is a substitution to alanine;
      (2) amino acid substitutions at positions 36 and 182 of SEQ ID NO:1, wherein the amino acid substitution at position 36 of SEQ ID NO:1 is a substitution to methionine and the amino acid substitution at position 182 of SEQ ID NO:1 is a substitution to aspartic acid;
      (3) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to phenylalanine;
      (4) amino acid substitutions at positions 216 and 261 of SEQ ID NO:1, wherein the amino acid substitution position 216 of SEQ ID NO:1 is a substitution to glycine and the amino acid substitution at position 261 of SEQ ID NO:1 is a substitution to asparagine;
      (5) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to serine; or
      (6) amino acid substitutions at positions 36 and 216 of SEQ ID NO:1, wherein the amino acid substitution at position 36 of SEQ ID NO:1 is a substitution to methionine and the amino acid substitution at position 216 of SEQ ID NO:1 is a substitution to glycine;
   b) growing the population of host cells in the presence of a neomycin phosphotransferase substrate to produce colonies; and
   c) selecting a colony of cells that grows in the presence of the neomycin phosphotransferase substrate.
D3. A method of making host cells comprising a second nucleotide sequence comprising:
   a) growing a population of hosts cells in the presence of a neomycin phosphotransferase substrate to produce colonies, wherein the population of host cells comprises a first nucleic acid sequence comprising (i) a first nucleotide sequence encoding the non-naturally occurring NPT, and (ii) a second nucleotide sequence comprising a transgene encoding a second protein or a non-coding RNA, wherein the non-naturally occurring NPT comprises an amino acid sequence of a wild-type neomycin phosphotransferase with:
      (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
      (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
      (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
      (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
      (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and
   b) selecting a colony of cells that grows in the presence of the neomycin phosphotransferase substrate.
D4. A method of making host cells comprising a second nucleotide sequence comprising:
a) growing a population of hosts cells in the presence of a neomycin phosphotransferase substrate to produce colonies, wherein the population of host cells comprises (i) a first nucleic acid sequence comprising a first nucleotide sequence encoding the non-naturally occurring NPT, and (ii) a second nucleic acid sequence comprising a transgene encoding a second protein or a non-coding RNA; wherein the non-naturally occurring NPT comprises the amino acid sequence of SEQ ID NO:1 with:
   (1) amino acid substitutions at positions 36 and 210 of SEQ ID NO:1, wherein the amino acid substitution at position 36 of SEQ ID NO:1 is a substitution to methionine and the amino acid substitution at position 210 of SEQ ID NO:1 is a substitution to alanine;
   (2) amino acid substitutions at positions 36 and 182 of SEQ ID NO:1, wherein the amino acid substitution at position 36 of SEQ ID NO:1 is a substitution to methionine and the amino acid substitution at position 182 of SEQ ID NO:1 is a substitution to aspartic acid;
   (3) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to phenylalanine;
   (4) amino acid substitutions at positions 216 and 261 of SEQ ID NO:1, wherein the amino acid substitution position 216 of SEQ ID NO:1 is a substitution to glycine and the amino acid substitution at position 261 of SEQ ID NO:1 is a substitution to asparagine;
   (5) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to serine; or
   (6) amino acid substitutions at positions 36 and 216 of SEQ ID NO:1, wherein the amino acid substitution at position 36 of SEQ ID NO:1 is a substitution to methionine and the amino acid substitution at position 216 of SEQ ID NO:1 is a substitution to glycine; and
b) selecting a colony of cells that grows in the presence of the neomycin phosphotransferase substrate.
D5. The method of embodiment D1 or D3, wherein the wild-type NPT comprises an amino acid sequence that is at least 80%, at least 90%, or at least 98% identical to SEQ ID NO:1.
D6. The method of embodiment D1 or D3, wherein the wild-type NPT comprises an amino acid sequence that is at least 60%, at least 65%, at least 70% or at least 75% identical to SEQ ID NO:1.
D7. The method of embodiment D2, wherein the non-naturally occurring NPT has attenuated neomycin phosphotransferase activity as a selectable marker as compared to wild-type NPT comprising the amino acid sequence of SEQ ID NO:1.
D8. The method of embodiment D1, D3, D4, D5 or D6, wherein bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT.
D9. The method of embodiment D8, wherein the bacterial cells are E. coli.
D10. The method of embodiment D1, D3, D4, D5 or D6, wherein mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT.
D11. The method of embodiment D10, wherein the mammalian cells are HEK293 cells, CHO cells, PER.C6 cells, murine NS0 cells, fibrosarcoma HT-1080 cells, murine Sp2/0 cells, BHK cells, or murine C127 cells.
D12. The method of embodiment D1, D3, D4, D5 or D6, wherein G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT.
D13. The method of embodiment D2 or D7, wherein bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT, wherein the wild-type NPT comprises the amino acid sequence of SEQ ID NO:1.
D14. The method of embodiment D13, wherein the bacterial cells are E. coli.
D15. The method of embodiment D2 or D7, wherein mammalian cells transfected with an expression vector comprising a nucleotide sequence encoding the non-naturally occurring NPT exhibit reduced colony formation as assessed by a colony formation assay after 2 weeks growth on tissue-culture plates in media containing 500 μg/mL geneticin (G418) relative to mammalian cells transfected with the same expression vector but comprising a nucleotide sequence encoding wild-type NPT, wherein the wild-type NPT comprises the amino acid sequence of SEQ ID NO: 1.
D16. The method of embodiment D15, wherein the mammalian cells are HEK293 cells, CHO cells, PER.C6 cells, murine NS0 cells, fibrosarcoma HT-1080 cells, murine Sp2/0 cells, BHK cells, or murine C127 cells.
D17. The method of embodiment D2 or D7, wherein G418 resistant colonies of mammalian cells transfected with an expression vector comprising a nucleotide sequence encoding the non-naturally occurring NPT are produced at frequencies ranging from 5.5% to 0.004% relative to G418 resistant colonies of mammalian cells transfected with the same expression vector but comprising a nucleotide sequence encoding wild-type NPT, wherein the wild-type NPT comprises the amino acid sequence of SEQ ID NO:1.
D18. The method of any one of embodiments D1, D3, D4, D5, D6 or D8 to D12, wherein the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.
D19. The method of any one of embodiments D1, D3, D4, D5, D6 or D8 to D12, wherein the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.
D20. The method of any one of embodiments D1, D3, D4, D5, D6 or D8 to D12, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine.
D21. The method of any one of embodiments D1, D3, D4, D5, D6 or D8-D12, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.
D22. The method of any one of embodiments D1, D3, D4, D5, D6 or D8 to D12, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine.
D23. The method of any one of embodiments D1, D3, D4, D5, D6 or D8 to D12, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 216 of SEQ ID NO:1 is a substitution to glycine.
D24. The method of any one of embodiment D2, D7 or D13-D17, wherein the non-naturally occurring NPT comprises the amino acid sequence of SEQ ID NO:38 (V36M, G210A).
D25. The method of any one of embodiment D2, D7 or D13-D17, wherein the non-naturally occurring NPT comprises the amino acid sequence of SEQ ID NO:39 (V36M, E182D).
D26. The method of any one of embodiment D2, D7 or D13-D17, wherein the non-naturally occurring NPT comprises the amino acid sequence of SEQ ID NO:40 (V36M, Y218F).
D27. The method of any one of embodiment D2, D7 or D13-D17, wherein the non-naturally occurring NPT comprises the amino acid sequence of SEQ ID NO:41 (D216G, D261N).
D28. The method of any one of embodiment D2, D7 or D13-D17, wherein the non-naturally occurring NPT comprises the amino acid sequence of SEQ ID NO:42 (V36M, Y218S).
D29. The method of any one of embodiment D2, D7 or D13-D17, wherein the non-naturally occurring NPT comprises the amino acid sequence of SEQ ID NO:43 (V36M, D216G).
D30. The method of any one of embodiments D1 to D29, wherein the population of host cells produces fewer colonies than a second population of host cells transfected or transformed with a second nucleic acid sequence and grown in the presence of the neomycin phosphotransferase substrate, wherein second nucleic acid sequence comprises a third nucleotide sequence encoding wild-type NPT protein and the second nucleotide sequence.
D31. The method of any one of embodiments D1 to D30, wherein the host cells are mammalian cells.
D32. The method of embodiment D31, wherein the mammalian cells are HEK293 cells, CHO cells, PER.C6 cells, murine NS0 cells, fibrosarcoma HT-1080 cells, murine Sp2/0 cells, BHK cells, or murine C127 cells.
D33. The method of any one of embodiments D1 to D29, wherein the cells are human cells.
D34. The method of any one of embodiments D1 to D33, which further comprises culturing the selected colony of cells.
D35. The method of any one of embodiments D1 to D34, wherein the neomycin phosphotransferase substrate is neomycin, kanamycin, or G418.
D36. The method of any one of embodiments D1 to D35, wherein the protein is a therapeutic protein or an antigen.
D37. The method of any one of embodiments D1 to D35, wherein the non-coding RNA is shRNA, miRNA, antisense RNA, guide RNA for Crispr nucleases, catalytic RNA, ribosomal RNA or tRNA.
D38. Host cells produced by the method of any one of embodiments D1 to D37.

In a fifth set of embodiments, provided are:
E1. A method for manufacturing a stable cell line expressing a therapeutic protein or enzyme comprising:
a) introducing one or more nucleic acid sequences into a population of host cells, wherein the one or more nucleic acid sequences comprise:
   (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with:
      (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
      (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
      (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
      (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
      (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and
         (ii) a second nucleic acid sequence encoding the therapeutic protein or enzyme;
      b) selecting a cell from the population of cells of step (a) that grows in the presence of G418; and
      c) culturing the selected cell to produce a stable cell line expressing the therapeutic protein or enzyme.
E2. A method for manufacturing a stable cell line expressing a therapeutic protein or enzyme comprising:
a) introducing one or more nucleic acid sequences into a population of host cells, wherein the one or more nucleic acid sequences comprise:
   (i) a first nucleic acid sequence encoding a non-naturally occurring neomycin phosphotransferase (NPT) with neomycin phosphotransferase activity, wherein the non-naturally occurring NPT comprises the amino acid sequence of SEQ ID NO:1 with:
      (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine;
      (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid;
      (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine;
      (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or
      (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and
         (ii) a second nucleic acid sequence encoding the therapeutic protein or enzyme;
      b) selecting a cell from the population of cells of step (a) that grows in the presence of G418; and
      c) culturing the selected cell to produce a stable cell line expressing the therapeutic protein or enzyme.
E3. The method of embodiment E1, wherein the non-naturally occurring NPT has attenuated neomycin phosphotransferase activity as a selectable marker as compared to wild-type NPT.
E4. The method of embodiment E1 or E3, wherein the wild-type NPT comprises an amino acid sequence that is at least 80%, at least 90%, or at least 98% identical to SEQ ID NO:1.
E5. The method of embodiment E1 or E3, wherein the wild-type NPT comprises an amino acid sequence that is at least 60%, at least 65%, at least 70%, or at least 75% identical to SEQ ID NO:1.
E6. The method of embodiment E2, wherein the non-naturally occurring NPT has attenuated neomycin phosphotransferase activity as a selectable marker as compared to wild-type NPT comprising the amino acid sequence of SEQ ID NO: 1.
E7. The method of embodiment E1, E3, E4, or E5, wherein the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.
E8. The method of embodiment E1, E3, E4, or E5, wherein the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.
E9. The method of embodiment E1, E3, E4, or E5, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine.
E10. The method of embodiment E1, E3, E4, or E5, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.
E11. The method of embodiment E1, E3, E4, or E5, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine.
E12. The method of embodiment E1, E3, E4, or E5, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
E13. The method of embodiment E2 or E6, wherein the non-naturally occurring NPT comprises the amino acid sequence of SEQ ID NO:38 (V36M, G210A).
E14. The method of embodiment E2 or E6, wherein the non-naturally occurring NPT comprises the amino acid sequence of SEQ ID NO:39 (V36M, E182D).
E15. The method of embodiment E2 or E6, wherein the non-naturally occurring NPT comprises the amino acid sequence of SEQ ID NO:40 (V36M, Y218F).
E16. The method of embodiment E2 or E6, wherein the non-naturally occurring NPT comprises the amino acid sequence of SEQ ID NO:41 (D216G, D261N).
E17. The method of embodiment E2 or E6, wherein the non-naturally occurring NPT comprises the amino acid sequence of SEQ ID NO:42 (V36M, Y218S).
E18. The method of embodiment E2 or E6, wherein the non-naturally occurring NPT comprises the amino acid sequence of SEQ ID NO:43 (V36M, D216G).
E19. The method of any one of embodiments E1 to E18, wherein the stable cell line is a mammalian cell line.
E20. The method of any one of embodiments E1 to E18, wherein the stable cell line is a human cell line.
E21. The method of any one of embodiments E1 to E18, wherein the stable cell line is a CHO, PER.C6, murine NS0, HEK293, fibrosarcoma HT-1080, murine Sp2/0, BHK, or murine C127 cell line.
E22. The method of any one of embodiments E1 to E21, wherein the stable cell line expresses the therapeutic protein.
E23. The method of embodiment E22, wherein the therapeutic protein is an antibody or antibody fragment.
E24. The method of any one of embodiments E1 to E21, wherein the stable cell line expresses the enzyme.
E25. A stable cell line produced by the method of any one of embodiments E1 to E24.

In a sixth set of embodiments, provided are:
F1. A method of making a virus producer cell line comprising:
   a) introducing one or more nucleic acid sequences into a population of host cells, wherein the one or more nucleic acid sequences comprise:
      (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with:
      (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
      (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
      (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
      (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
      (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and
         (ii) a second nucleic acid sequence encoding one or more viral proteins, wherein the one or more viral proteins includes a capsid protein, an envelope protein, a viral protein necessary for replication, or a combination thereof;
   b) selecting a cell from the population of cells that grows in the presence in of a neomycin phosphotransferase substrate; and
   c) propagating the selected cell to produce a virus producer cell line.
F2. A method of making a virus producer cell line comprising:
   a) introducing one or more nucleic acid sequences into a population of host cells, wherein the one or more nucleic acid sequences comprise:
      (i) a first nucleic acid sequence encoding a non-naturally occurring neomycin phosphotransferase (NPT) with neomycin phosphotransferase activity, wherein the non-naturally occurring NPT comprises the amino acid sequence of SEQ ID NO:1 with:
      (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine;
      (2) amino acid substitutions at amino acid residues 36 and 182 of SEQ ID NO:1, wherein the amino acid substitution at the amino acid residue 36 of SEQ ID NO:1 is a substitution to methionine and the amino acid substitution at amino acid residue 182 of SEQ ID NO:1 is a substitution to aspartic acid;
      (3) amino acid substitutions at amino acid residues 36 and 218 of SEQ ID NO:1, wherein the amino acid substitution at the amino acid residue 36 of SEQ ID NO:1 is a substitution to methionine and the amino acid substitution at amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue 216 of SEQ ID NO:1 is a substitution to glycine and the amino acid substitution at amino acid residue 261 of SEQ ID NO:1 is a substitution to asparagine;
      (5) amino acid substitutions at amino acid residues 36 and 218 of SEQ ID NO:1, wherein the amino acid substitution at the amino acid residue 36 of SEQ ID NO:1 is a substitution to methionine and the amino acid substitution at amino acid residue 218 of SEQ ID NO:1 is a substitution to serine; or
      (6) amino acid substitutions at amino acid residues 36 and 216 of SEQ ID NO:1, wherein the amino acid substitution at the amino acid residue 36 of SEQ ID NO:1 is a substitution to methionine and the amino acid substitution at amino acid residue 216 of SEQ ID NO:1 is a substitution to glycine; and
         (ii) a second nucleic acid sequence encoding one or more viral proteins, wherein the one or more viral proteins includes a capsid protein, an envelope protein, a viral protein necessary for replication, or a combination thereof;
      b) selecting a cell from the population of cells that grows in the presence in of a neomycin phosphotransferase substrate; and
      c) propagating the selected cell to produce a virus producer cell line.
F3. The method of embodiment F1, wherein the non-naturally occurring NPT has attenuated neomycin phosphotransferase activity as a selectable marker as compared to wild-type NPT.
F4. The method of embodiment F1 or F3, wherein the wild-type NPT comprises an amino acid sequence that is at least 80%, at least 90%, or at least 98% identical to SEQ ID NO:1.
F5. The method of embodiment F1 or F3, wherein the wild-type NPT comprises an amino acid sequence that is at least 60%, at least 65%, at least 70%, or at least 75% identical to SEQ ID NO:1.
F6. The method of embodiment F2, wherein the non-naturally occurring NPT has attenuated neomycin phosphotransferase activity as a selectable marker as compared to wild-type NPT comprising the amino acid sequence of SEQ ID NO:1.
F7. The method of embodiment F1, F3, F4, or F5, wherein the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.
F8. The method of embodiment F1, F3, F4, or F5, wherein the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.
F9. The method of embodiment F1, F3, F4, or F5, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine.
F10. The method of embodiment F1, F3, F4, or F5, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.
F11. The method of embodiment F1, F3, F4, or F5, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine.
F12. The method of embodiment F1, F3, F4, or F5, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
F13. The method of embodiment F2 or F6, wherein the non-naturally occurring NPT comprises the amino acid sequence of SEQ ID NO:38 (V36M, G210A).
F14. The method of embodiment F2 or F6, wherein the non-naturally occurring NPT comprises the amino acid sequence of SEQ ID NO:39 (V36M, E182D).
F15. The method of embodiment F2 or F6, wherein the non-naturally occurring NPT comprises the amino acid sequence of SEQ ID NO:40 (V36M, Y218F).
F16. The method of embodiment F2 or F6, wherein the non-naturally occurring NPT comprises the amino acid sequence of SEQ ID NO:41 (D216G, D261N).
F17. The method of embodiment F2 or F6, wherein the non-naturally occurring NPT comprises the amino acid sequence of SEQ ID NO:42 (V36M, Y218S).
F18. The method of embodiment F2 or F6, wherein the non-naturally occurring NPT comprises the amino acid sequence of SEQ ID NO:43 (V36M, D216G).
F19. The method of any one of embodiments F1 to F18, wherein the cell line is a mammalian cell line.
F20. The method of any one of embodiments F1 to F18, wherein the cell line is a human cell line.
F21. The method of any one of embodiments F1 to F18, wherein the cell line is a CHO, PER.C6, murine NS0, HEK293, fibrosarcoma HT-1080, murine Sp2/0, BHK, or murine C127 cell line.
F22. The method of any one of embodiments F1 to F21, wherein the one or more viral proteins includes an AAV capsid protein.
F23. The method of any one of embodiments F1 to F21, wherein the one or more viral proteins includes an AAV capsid protein and AAV rep protein.
F24. The method of any one of embodiment F1 to F21, wherein the one or more viral proteins includes an envelope protein.
F25. The method of any one of embodiment F1 to F21, wherein the one or more viral proteins includes adenovirus E1 region proteins required for adenovirus replication.
F26. The method of any one of embodiment F1 to F21, wherein the one or more viral proteins includes a retroviral envelope protein.
F27. The method of any one of embodiment F1 to F21, wherein the one or more viral proteins includes a retroviral gag protein.
F28. The method of any one of embodiment F1 to F21, wherein the one or more viral proteins includes a retroviral reverse transcriptase.
F29. The method of any one of embodiment F1 to F21, wherein the one or more viral proteins includes a retroviral envelope protein, gag protein and reverse transcriptase.
F30. A virus producer cell line made by the method of any one of embodiments F1 to F29.
F31. A virus producer cell line comprising one or more nucleic acid sequences, wherein the one or more nucleic acid sequences comprise:
   (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with:
      (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
      (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 182 of SEQ ID NO:1 is a substitution to aspartic acid;
      (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
      (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
      (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and
         (ii) a second nucleic acid sequence encoding one or more viral proteins, wherein the one or more viral proteins includes a capsid protein, an envelope protein, a viral protein necessary for replication, or a combination thereof.
F32. A virus producer cell line comprising one or more nucleic acid sequences, wherein the one or more nucleic acid sequences comprise:
   (i) a first nucleic acid sequence encoding a non-naturally occurring neomycin phosphotransferase (NPT) with neomycin phosphotransferase activity, wherein the non-naturally occurring NPT comprises the amino acid sequence of SEQ ID NO:1 with:
      (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine;
      (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid;
      (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine;
      (4) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine;
      (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or
      (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and
         (ii) a second nucleic acid sequence encoding one or more viral proteins, wherein the one or more viral proteins includes a capsid protein, an envelope protein, a viral protein necessary for replication, or a combination thereof.
F33. The virus producer cell line of embodiment F31, wherein the non-naturally occurring NPT has attenuated neomycin phosphotransferase activity as a selectable marker as compared to wild-type NPT.
F34. The virus producer cell line of embodiment F31 or F33, wherein the wild-type NPT comprises an amino acid sequence that is at least 80%, at least 90%, or at least 98% identical to SEQ ID NO:1.
F35. The virus producer cell line of embodiment F31 or F33, wherein the wild-type NPT comprises an amino acid sequence that is at least 60%, at least 65%, at least 70%, or at least 75% identical to SEQ ID NO:1.
F36. The virus producer cell line of embodiment F32, wherein the non-naturally occurring NPT has attenuated neomycin phosphotransferase activity as a selectable marker as compared to wild-type NPT comprising the amino acid sequence of SEQ ID NO:1.
F37. The virus producer cell line of embodiment F31, F33, F34, or F35, wherein the non-naturally occurring NPT comprises amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine.
F38. The virus producer cell line of embodiment F31, F33, F34, or F35, wherein the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid.
F39. The virus producer cell line of embodiment F31, F33, F34, or F35, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine.
F40. The virus producer cell line of embodiment F31, F33, F34, or F35, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine.
F41. The virus producer cell line of embodiment F31, F33, F34, or F35, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine.
F42. The virus producer cell line of embodiment F31, F33, F34, or F35, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.
F43. The virus producer cell line of embodiment F32 or F36, wherein the non-naturally occurring NPT comprises the amino acid sequence of SEQ ID NO:38 (V36M, G210A).
F44. The virus producer cell line of embodiment F32 or F36, wherein the non-naturally occurring NPT comprises the amino acid sequence of SEQ ID NO:39 (V36M, E182D).
F45. The virus producer cell line of embodiment F32 or F36, wherein the non-naturally occurring NPT comprises the amino acid sequence of SEQ ID NO:40 (V36M, Y218F).
F46. The virus producer cell line of embodiment F32 or F36, wherein the non-naturally occurring NPT comprises the amino acid sequence of SEQ ID NO:41 (D216G, D261N).
F47. The virus producer cell line of embodiment F32 or F36, wherein the non-naturally occurring NPT comprises the amino acid sequence of SEQ ID NO:42 (V36M, Y218S).
F48. The virus producer cell line of embodiment F32 or F36, wherein the non-naturally occurring NPT comprises the amino acid sequence of SEQ ID NO:43 (V36M, D216G).
F49. The virus producer cell line of any one of embodiments F31 to F48, wherein the cell line is a mammalian cell line.
F50. The virus producer cell line of any one of embodiments F31 to F48, wherein the cell line is a human cell line.
F51. The virus producer cell line of any one of embodiments F31 to F48, wherein the cell line is a CHO, PER.C6, murine NS0, HEK293, fibrosarcoma HT-1080, murine Sp2/0, BHK, or murine C127 cell line.
F52. The virus producer cell line of any one of embodiments F31 to F51, wherein the one or more viral proteins includes an AAV capsid protein.
F53. The virus producer cell line of any one of embodiments F31 to F51, wherein the one or more viral proteins includes an AAV capsid protein and AAV rep protein.
F54. The virus producer cell line of any one of embodiment F31 to F51, wherein the one or more viral proteins includes an envelope protein.
F55. The virus producer cell line of any one of embodiment F31 to F51, wherein the one or more viral proteins includes adenovirus E1 region proteins required for adenovirus replication.
F56. The virus producer cell line of any one of embodiment F31 to F51, wherein the one or more viral proteins includes a retroviral envelope protein.
F57. The virus producer cell line of any one of embodiment F31 to F51, wherein the one or more viral proteins includes a retroviral gag protein.
F58. The virus producer cell line of any one of embodiment F31 to F51, wherein the one or more viral proteins includes a retroviral reverse transcriptase.
F59. The virus producer cell line of any one of embodiment F31 to F51, wherein the one or more viral proteins includes a retroviral envelope protein, gag protein and reverse transcriptase.

In a seventh set of embodiments, provided are: G1. A method for manufacturing a cell line expressing an antigen comprising:
a) introducing one or more nucleic acid sequences into a population of host cells, wherein the one or more nucleic acid sequences comprise:
   (i) a first nucleic acid sequence encoding a non-naturally occurring neomycin phosphotransferase (NPT) with neomycin phosphotransferase activity, wherein the non-naturally occurring neomycin NPT comprises an amino acid sequence of a wild-type neomycin phosphotransferase with:
      (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
      (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
      (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
      (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
      (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine; and
         (ii) a second nucleic acid sequence encoding an antigen;
      b) selecting a cell from the population of cells of step (a) that grows in the presence of G418; and
      c) culturing the selected cell to produce a cell line expressing the antigen.
G2. A method for manufacturing a cell line expressing an antigen comprising:
a) introducing one or more nucleic acid sequences into a population of host cells, wherein the one or more nucleic acid sequences comprise:
   (i) a first nucleic acid sequence encoding a non-naturally occurring neomycin phosphotransferase (NPT) with neomycin phosphotransferase activity, wherein the non-naturally occurring neomycin NPT comprises the amino acid sequence of SEQ ID NO:1 with:
      (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine;
      (2) amino acid substitutions at amino acid residues 36 and 182 of SEQ ID NO:1, wherein the amino acid substitution at amino acid residue 36 of SEQ ID NO:1 is a substitution to methionine and the amino acid substitution at amino acid residue 182 of SEQ ID NO:1 is a substitution to aspartic acid;
      (3) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to phenylalanine;
      (4) amino acid substitutions at amino acid residues 216 and 261 of SEQ ID NO:1, wherein the amino acid substitution at amino acid residue 216 of SEQ ID NO:1 is a substitution to glycine and the amino acid substitution at amino acid residue 261 of SEQ ID NO:1 is a substitution to asparagine;
      (5) amino acid substitutions at amino acid residues 36 and 218 of SEQ ID NO:1, wherein the amino acid substitution at amino acid residue 36 of SEQ ID NO:1 is a substitution to methionine and the amino acid substitution at amino acid residue 218 of SEQ ID NO:1 is a substitution to serine; or
      (6) amino acid substitutions at amino acid residues 36 and 216 of SEQ ID NO:1, wherein the amino acid substitution at amino acid residue 36 of SEQ ID NO:1 is a substitution to methionine and the amino acid substitution at amino acid residue 216 of SEQ ID NO:1 is a substitution to glycine; and
         (ii) a second nucleic acid sequence encoding an antigen;
      b) selecting a cell from the population of cells of step (a) that grows in the presence of G418; and
      c) culturing the selected cell to produce a cell line expressing the antigen.
G3. The method of embodiment G1, wherein the non-naturally occurring NPT has attenuated neomycin phosphotransferase activity as a selectivity marker as compared to wild-type NPT.
G4. The method of embodiment G1 or G3, wherein the wild-type NPT comprises an amino acid sequence that is at least 80%, at least 90%, or at least 98% identical to SEQ ID NO:1.
G5. The method of embodiment G1, wherein the wild-type NPT comprises an amino acid sequence that is at least 60%, at least 65%, at least 70%, or at least 75% identical to SEQ ID NO:1.
G6. The method of embodiment G2, wherein the non-naturally occurring NPT has attenuated neomycin phosphotransferase activity as a selectivity marker as compared to wild-type NPT comprising the amino acid sequence of SEQ ID NO:1.
G7. The method of embodiment G1, G3, G4, or G5, wherein the non-naturally occurring NPT comprises the amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 210 of SEQ ID NO:1 is a substitution to alanine.
G8. The method of embodiment G1, G3, G4, or G5, wherein the non-naturally occurring NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 182 of SEQ ID NO:1 is a substitution to aspartic acid.
G9. The method of embodiment G1, G3, G4, or G5, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to phenylalanine.
G10. The method of embodiment G1, G3, G4, or G5, wherein the non-naturally occurring NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at amino acid residue corresponding to amino acid residue 261 of SEQ ID NO:1 is a substitution to asparagine.
G11. The method of embodiment G1, G3, G4, or G5, wherein the non-naturally occurring NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to serine.
G12. The method of embodiment G1, G3, G4, or G5, wherein the non-naturally occurring NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 216 of SEQ ID NO:1 is a substitution to glycine.
G13. The method of embodiment G2 or G6, wherein the non-naturally occurring NPT comprises the amino acid sequence of SEQ ID NO:38 (V36M, G210A).
G14. The method of embodiment G2 or G6, wherein the non-naturally occurring NPT comprises the amino acid sequence of SEQ ID NO:39 (V36M, E182D).
G15. The method of embodiment G2 or G6, wherein the non-naturally occurring NPT comprises the amino acid sequence of SEQ ID NO:40 (V36M, Y218F).
G16. The method of embodiment G2 or G6, wherein the non-naturally occurring NPT comprises the amino acid sequence of SEQ ID NO:41 (D216G, D261N).
G17. The method of embodiment G2 or G6, wherein the non-naturally occurring NPT comprises the amino acid sequence of SEQ ID NO:42 (V36M, Y218S).
G18. The method of embodiment G2 or G6, wherein the non-naturally occurring NPT comprises the amino acid sequence of SEQ ID NO:43 (V36M, D216G).
G19. The method of any one of embodiments G1 to G18, wherein the cell line is a mammalian cell line.
G20. The method of any one of embodiments G1 to G18, wherein the cell line is a human cell line.
G21. The method of any one of embodiments G1 to G18, wherein the cell line is a CHO, PER.C6, murine NS0, HEK293, fibrosarcoma HT-1080, murine Sp2/0, BHK, or murine C127 cell line.
G22. The method of any one of embodiments G1 to G21, wherein the antigen is a viral antigen, a bacterial antigen, or a fungal antigen.
G23. The method of any one of embodiments G1 to G21, wherein the antigen is a cancer antigen.
G24. An antigen producing cell line made by the method of any one of embodiments G1 to G23.
G25. An antigen producing cell line comprising one or more nucleic acid sequences, wherein the one or more nucleic acid sequences comprise:
   (i) a first nucleic acid sequence encoding a non-naturally occurring neomycin phosphotransferase (NPT) with neomycin phosphotransferase activity, wherein the non-naturally occurring NPT comprises an amino acid sequence of a wild-type neomycin phosphotransferase with:
      (1) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 210 of SEQ ID NO:1 is a substitution to alanine;
      (2) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 182 of SEQ ID NO:1 is a substitution to aspartic acid;
      (3) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at amino acid residue corresponding to amino acid residue 261 of SEQ ID NO:1 is a substitution to asparagine;
      (5) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to serine; or
      (6) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 216 of SEQ ID NO:1 is a substitution to glycine; and
         (ii) a second nucleic acid sequence encoding one or more antigens.
G26. An antigen producing cell line comprising one or more nucleic acid sequences, wherein the one or more nucleic acid sequences comprise:
   (i) a first nucleic acid sequence encoding a non-naturally occurring neomycin phosphotransferase (NPT) with neomycin phosphotransferase activity, wherein the non-naturally occurring NPT comprises the amino acid sequence of SEQ ID NO: 1 with:
      (1) amino acid substitutions at amino acid residues 36 and 210 of SEQ ID NO:1, wherein the amino acid substitution at amino acid residue 36 of SEQ ID NO:1 is a substitution to methionine and the amino acid substitution at amino acid residue 210 of SEQ ID NO:1 is a substitution to alanine;
      (2) amino acid substitutions at amino acid residues 36 and 182 of SEQ ID NO:1, wherein the amino acid substitution at the amino acid residue 36 of SEQ ID NO:1 is a substitution to methionine and the amino acid substitution at amino acid residue 182 of SEQ ID NO:1 is a substitution to aspartic acid;
      (3) amino acid substitutions at amino acid residues 36 and 218 of SEQ ID NO:1, wherein the amino acid substitution at the amino acid residue 36 of SEQ ID NO:1 is a substitution to methionine and the amino acid substitution at amino acid residue 218 of SEQ ID NO:1 is a substitution to phenylalanine;
      (4) amino acid substitutions at amino acid residues corresponding to amino acid residues 216 and 261 of SEQ ID NO:1, wherein the amino acid substitution at the amino acid residue 216 of SEQ ID NO:1 is a substitution to glycine and the amino acid substitution at amino acid residue 261 of SEQ ID NO:1 is a substitution to asparagine;
      (5) amino acid substitutions at amino acid residues 36 and 218 of SEQ ID NO:1, wherein the amino acid substitution at the amino acid residue 36 of SEQ ID NO:1 is a substitution to methionine and the amino acid substitution at amino acid residue 218 of SEQ ID NO:1 is a substitution to serine; or
      (6) amino acid substitutions at amino acid residues 36 and 216 of SEQ ID NO:1, wherein the amino acid substitution at the amino acid residue 36 of SEQ ID NO:1 is a substitution to methionine and the amino acid substitution at amino acid residue 216 of SEQ ID NO:1 is a substitution to glycine; and
         (ii) a second nucleic acid sequence encoding one or more antigens.
G27. The antigen producing cell line of embodiment G25, wherein the non-naturally occurring NPT has attenuated neomycin phosphotransferase activity as a selectivity marker as compared to wild-type NPT.
G28. The antigen producing cell line of embodiment G25 or G27, wherein the wild-type NPT comprises an amino acid sequence that is at least 80%, at least 90%, or at least 98% identical to SEQ ID NO:1.
G29. The antigen producing cell line of embodiment G25 or G27, wherein the wild-type NPT comprises an amino acid sequence that is at least 60%, at least 65%, at least 70%, or at least 65% identical to SEQ ID NO:1.
G30. The antigen producing cell line of embodiment G26, wherein the non-naturally occurring NPT has attenuated neomycin phosphotransferase activity as a selectivity marker as compared to wild-type NPT comprising the amino acid sequence of SEQ ID NO:1.
G31. The antigen producing cell line of embodiment G25, G27, G28 or G29, wherein the NPT comprises the amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 210 of SEQ ID NO:1 is a substitution to alanine.
G32. The antigen producing cell line of embodiment G25, G27, G28 or G29, wherein the NPT comprises amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 182 of SEQ ID NO:1 is a substitution to aspartic acid.
G33. The antigen producing cell line of embodiment G25, G27, G28 or G29, wherein the NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to phenylalanine.
G34. The antigen producing cell line of embodiment G25, G27, G28 or G29, wherein the NPT comprises amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at amino acid residue corresponding to amino acid residue 261 of SEQ ID NO:1 is a substitution to asparagine.
G35. The antigen producing cell line of embodiment G25, G27, G28 or G29, wherein the NPT comprises amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 218 of SEQ ID NO:1 is a substitution to serine.
G36. The antigen producing cell line of embodiment G25, G27, G28 or G29, wherein the NPT comprises amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at amino acid residue corresponding to amino acid residue 216 of SEQ ID NO:1 is a substitution to glycine.
G37. The antigen producing cell line of embodiment G26 or G30, wherein the non-naturally occurring NPT comprises the amino acid sequence of SEQ ID NO:38 (V36M, G210A).
G38. The antigen producing cell line of embodiment G26 or G30, wherein the non-naturally occurring NPT comprises the amino acid sequence of SEQ ID NO:39 (V36M, E182D).
G39. The antigen producing cell line of embodiment G26 or G30, wherein the non-naturally occurring NPT comprises the amino acid sequence of SEQ ID NO:40 (V36M, Y218F).
G40. The antigen producing cell line of embodiment G26 or G30, wherein the non-naturally occurring NPT comprises the amino acid sequence of SEQ ID NO:41 (D216G, D261N).
G41. The antigen producing cell line of embodiment G26 or G30, wherein the non-naturally occurring NPT comprises the amino acid sequence of SEQ ID NO:42 (V36M, Y218S).
G42. The antigen producing cell line of embodiment G26 or G30, wherein the non-naturally occurring NPT comprises the amino acid sequence of SEQ ID NO:43 (V36M, D216G).
G43. The antigen producing cell line of any one of embodiments G25 to G42, wherein the cell line is a mammalian cell line.
G44. The antigen producing cell line of any one of embodiments G25 to G42, wherein the cell line is a human cell line.
G45. The antigen producing cell line of any one of embodiments G25 to G42, wherein the cell line is a CHO, PER.C6, murine NS0, HEK293, fibrosarcoma HT-1080, murine Sp2/0, BHK, or murine C127 cell line.
G46. The antigen producing cell line of any one of embodiments G25 to G45, wherein the one or more antigens is a viral antigen, a bacterial antigen, or a fungal antigen.
G47. The antigen producing cell line of any one of embodiments G25 to G45, wherein the one or more antigens is a cancer antigen.

In an eighth set of embodiments, provided are: H1. A selectable marker means for conferring resistance to kanamycin when introduced into a bacterial cell, and to G418 when introduced into a mammalian cell.
H2. The selectable maker means of embodiment H1 comprising a nucleic acid sequence of SEQ ID NO:20.
H3. The selectable maker means of embodiment H1 comprising a nucleic acid sequence of SEQ ID NO:32.
H4. The selectable maker means of embodiment H1 comprising a nucleic acid sequence of SEQ ID NO:33.
H5. The selectable maker means of embodiment H1 comprising a nucleic acid sequence of SEQ ID NO:34.
H6. The selectable maker means of embodiment H1 comprising a nucleic acid sequence of SEQ ID NO:36.
H7. The selectable maker means of embodiment H1 comprising a nucleic acid sequence of SEQ ID NO:37.
H8. A method for manufacturing a producer cell line comprising:
a) transforming a bacterial or mammalian cell with an expression vector comprising a nucleic acid sequence encoding one or more viral proteins and a means for growing in the presence of kanamycin if the transformed cell is a bacterial cell and for growing in the presence of G418 if the transformed cell is a mammalian cell to make a transformed cell; and
b) culturing the transformed cell in the presence of kanamycin or G418 to obtain a producer cell line, wherein the producer cell line expresses one or more viral proteins from AAV, adenovirus, retrovirus, lentivirus, herpes simplex virus, vaccinia virus or baculovirus.
H9. A method for selecting a cell with stable chromosomal integration of an exogenous nucleic acid sequence comprising:
a) transforming a population of eukaryotic cells with an exogenous nucleic acid sequence comprising a means for growing in the presence of G418;
b) culturing the population of transformed cells in the presence of G418 to produce colonies of transformed cells capable of growing in the presence of G418; and
c) selecting a cell from a colony produced in step (b) to obtain a cell with a stable chromosomal integration of the exogenous nucleic acid.
H10. The method of embodiment H9, wherein the exogenous nucleic acid sequence further comprises a transgene, and the selected cell expresses the transgene.
H11. The method of embodiment H9, wherein the exogenous nucleic acid sequence disrupts expression of a gene endogenous to the selected cell.
H12. A method for selecting a mammalian cell with a stable episome comprising: a) transforming a population of mammalian cells with a plasmid comprising a means for growing in the presence of G418;
b) culturing the population of transformed cells in the presence of G418 to produce colonies of transformed cells capable of growing in the presence of G418; and
c) selecting a cell from a colony produced in step (b) to obtain a cell with a stable episome comprising the plasmid.
H13. The method of embodiment H12, wherein the plasmid further comprises an EBNA1 OriP nucleic acid sequence and the selected cell expresses EBNA1.
H14. A method for selecting a mammalian cell transiently expressing a transgene comprising:
a) introducing into a population of mammalian cells a nucleic acid encoding a transgene and a means for growing in the presence of G418;
b) culturing the population of mammalian cells in the presence of G418 for 48-72 hours; and
c) selecting a mammalian cell from the cultured population of mammalian cells that grows in the presence of G418, wherein the selected mammalian cell transiently expresses the transgene.
H15. The method of embodiment H14, wherein the transgene comprises nucleic acid sequences encoding a Crispr endonuclease or a Crispr guide RNA.
H16. The method of any one of embodiments H8 to H15, wherein the means is nucleotide sequence encoding a non-naturally occurring neomycin phosphotransferase comprising the amino acid sequence selected from the group of SEQ ID NO: 38, 39, 40, 41, 42 or 43.

### 10. SEQUENCES DISCLOSED HEREIN

The following table provides a summary of sequence identification numbers assigned to sequences described herein:

| SEQ ID NO | Type of sequence | Description |
|---|---|---|
| 1 | Amino acid | Wild-type version Neomycin phosphotransferase |
| | | (aminoglycoside phosphotransferase 3′-IIa) |
| 2 | Nucleic acid | P313 WT Vector |
| 3 | Nucleic acid | Human Elongation Factor Alpha Promoter |
| 4 | Nucleic acid | mCherry Coding region |
| 5 | Nucleic acid | SV40 polyadenylation signal |
| 6 | Nucleic acid | Wild-type Neomycin phosphotransferase |
| 7 | Nucleic acid | Mouse Phosphoglycerate kinase promoter |
| 8 | Nucleic acid | E. Coli laczya promoter |
| 9 | Nucleic acid | Herpes Simplex Virus polyadenylation signal |
| 10 | Nucleic acid | ampicillin resistance gene |
| 11 | Nucleic acid | pUC57 plasmid replication origin |
| 12 | Nucleic acid | P614 Neo ORF |
| 13 | Nucleic acid | P615 Neo ORF |
| 14 | Nucleic acid | P616 Neo ORF |
| 15 | Nucleic acid | P623 Neo ORF |
| 16 | Nucleic acid | P624 Neo ORF |
| 17 | Nucleic acid | P626 Neo ORF |
| 18 | Amino acid | APH(6)-Ia depicted in FIGS. 6A-6B |
| 19 | Amino acid | APH(6)-Ib depicted in FIGS. 6A-6B |
| 20 | Nucleic acid | P629 Neo ORF (D216G D26IN) NPT |
| 21 | Nucleic acid | P641 Neo ORF |
| 22 | Nucleic acid | P642 Neo ORF |
| 23 | Nucleic acid | P643 Neo ORF |
| 24 | Nucleic acid | P675 Neo ORF |
| 25 | Nucleic acid | P676 Neo ORF |
| 26 | Nucleic acid | P677 Neo ORF |
| 27 | Nucleic acid | P678 Neo ORF |
| 28 | Nucleic acid | P679 Neo ORF |
| 29 | Nucleic acid | P680 Neo ORF |
| 30 | Nucleic acid | P681 Neo ORF |
| 31 | Nucleic acid | P682 Neo ORF |
| 32 | Nucleic acid | P683 Neo ORF, (V36M G210A) NPT |
| 33 | Nucleic acid | P684 Neo ORF, (V36M Y218S) NPT |
| 34 | Nucleic acid | P685 Neo ORF, (V36M Y218F) NPT |
| 35 | Nucleic acid | P686 Neo ORF |
| 36 | Nucleic acid | P687 Neo ORF, (V36M E182D) NPT |
| 37 | Nucleic acid | P688 Neo ORF, (V36M D216G) NPT |
| 38 | Amino acid | P683 (V36M G210A) NPT |
| 39 | Amino acid | P687 (V36M E182D) NPT |
| 40 | Amino acid | P685 (V36M Y218F) NPT |
| 41 | Amino acid | P629 (D216G D261N) NPT |
| 42 | Amino acid | P684 (V36M Y218S) NPT |
| 43 | Amino acid | P688 (V36M D216G) NPT |
| 44 | Amino acid | Wild-type NPT (GenBank No. U00004) |
| 45 | Amino acid | APH(6)-Ic depicted in FIGS. 6A-6B |
| 46 | Amino acid | APH(6)-Id depicted in FIGS. 6A-6B |
| 47 | Amino acid | APH(3′)-IIIa depicted in FIGS. 6A-6B |
| 48 | Amino acid | APH(3′)-VIIa depicted in FIGS. 6A-6B |
| 49 | Amino acid | APH(3′)-VIa depicted in FIGS. 6A-6B |
| 50 | Amino acid | APH(3′)-IVa depicted in FIGS. 6A-6B |
| 51 | Amino acid | APH(3′)-Ia depicted in FIGS. 6A-6B |
| 52 | Amino acid | APH(3′)-Ic depicted in FIGS. 6A-6B |
| 53 | Amino acid | APH(3′)-Ib depicted in FIGS. 6A-6B |
| 54 | Amino acid | APH(3′)-IIa depicted in FIGS. 6A-6B |
| 55 | Amino acid | APH(3′)-Vb depicted in FIGS. 6A-6B |
| 56 | Amino acid | APH(3′)-Va depicted in FIGS. 6A-6B |
| 57 | Amino acid | APH(3′)-Vc depicted in FIGS. 6A-6B |
| 58 | Amino acid | APH(3″)-Ia depicted in FIGS. 6A-6B |
| 59 | Amino acid | APH(3″)-Ib depicted in FIGS. 6A-6B |
| 60 | Amino acid | APH(2″)-Ia depicted in FIGS. 6A-6B |
| 61 | Amino acid | APH(4)-Ib depicted in FIGS. 6A-6B |
| 62 | Amino acid | APH(4)-Ia depicted in FIGS. 6A-6B |

Protein sequence for the wild-type Neomycin phosphotransferase protein
>SEQ ID NO: 1
MIEQDGLHAGSPAAWVERLFGYDWAQQTIGCSDAAVFRLSAQGRPVLFVKTDLSGALNELQDEAARLSWLATTGVPCAAVLDVVTEAGR
DWLLLGEVPGQDLLSSHLAPAEKVSIMADAMRRLHTLDPATCPFDHQAKHRIERARTRMEAGLVDQDDLDEEHQGLAPAELFARLKASM
PDGEDLVVTHGDACLPNIMVENGRFSGFIDCGRLGVADRYQDIALATRDIAEELGGEWADRFLVLYGIAAPDSQRIAFYRLLDEFF,
P313 WT Vector
>SEQ ID NO: 2
taactataacggtcctaaggtagcgaacctgcaggcagctgcgcgctcgctcgctcactgaggccgcccgggcaaagcccgggcgtcgg
gcgacctttggtcgcccggcctcagtgagcgagcgagcgcgcagagagggagtggccaactccatcactaggggttcctgcggccaatt
cagtcgataactataacggtcctaaggtagcgatttaaatacgcgctctcttaaggtagccgtgaggctccggtgcccgtcagtgggca
gagcgcacatcgcccacagtccccgagaagttggggggaggggtcggcaattgaaccggtgcctagagaaggtggcgcggggtaaactg
ggaaagtgatgtcgtgtactggctccgcctttttcccgaggggggggagaaccgtatataagtgcagtagtcgccgtgaacgttctttt
tcgcaacgggtttgccgccagaacacaggtaagtgccgtgtgtggttcccgcgggcctggcctctttacgggttatggcccttgcgtgc
cttgaattacttccacgcccctggctgcagtacgtgattcttgatcccgagcttcgggttggaagtgggtgggagagttcgaggccttg
cgcttaaggagccccttcgcctcgtgcttgagttgaggcctggcctgggcgctggggccgccgcgtgcgaatctggtggcaccttcgcg
cctgtctcgctgctttcgataagtctctagccatttaaaatttttgatgacctgctgcgacgctttttttctggcaagatagtcttgta
aatgcgggccaagatctgcacactggtatttcggtttttggggccgcgggcggcgacggggcccgtgcgtcccagcgctcatgttcggc
gaggcggggcctgcgagcgcggccaccgagaatcggacgggggtagtctcaagctggccggcctgctctggtgcctggcctcgcgccgc
cgtgtatcgccccgccctgggcggcaaggctggcccggtcggcaccagttgcgtgagcggaaagatggccgcttcccggccctgctgca
gggagctcaaaatggaggacgcggcgctcgggagagcggggggtgagtcacccacacaaaggaaaagggcctttccgtcctcagccgtc
gcttcatgtgactccacggagtaccgggcgccgtccaggcacctcgattagttctcgagcttttggagtacgtcgtctttaggttgggg
ggaggggttttatgcgatggagtttccccacactgagtgggtggagactgaagttaggccagcttggcacttgatgtaattctccttgg
aatttgccctttttgagtttggatcttggttcattctcaagcctcagacagtggttcaaagtttttttcttccatttcaggtgtcgtga
ggcgcgccgccaccatggtgagcaagggcgaggaggataacatggccatcatcaaggagttcatgcgcttcaaggtgcacatggagggc
tccgtgaacggccacgagttcgagatcgagggcgagggcgagggccgcccctacgagggcacccagaccgccaagctgaaggtgaccaa
gggtggccccctgcccttcgcctgggacatcctgtcccctcagttcatgtacggctccaaggcctacgtgaagcaccccgccgacatcc
ccgactacttgaagctgtccttccccgagggcttcaagtgggagcgcgtgatgaacttcgaggacggcggcgtggtgaccgtgacccag
gactcctccctgcaggacggcgagttcatctacaaggtgaagctgcgcggcaccaacttcccctccgacggccccgtaatgcagaagaa
gaccatgggctgggaggcctcctccgagcggatgtaccccgaggacggcgccctgaagggcgagatcaagcagaggctgaagctgaagg
acggcggccactacgacgctgaggtcaagaccacctacaaggccaagaagcccgtgcagctgcccggcgcctacaacgtcaacatcaag
ttggacatcacctcccacaacgaggactacaccatcgtggaacagtacgaacgcgccgagggccgccactccaccggcggcatggacga
gctgtacaagtagtctagagatacattgatgagtttggacaaaccacaactagaatgcagtgaaaaaaatgctttatttgtgaaatttg
tgatgctattgctttatttgtaaccattataagctgcaataaacaagttaacaacaacaattgcattcattttatgtttcaggttcagg
gggaggtgtgggaggttttttaaagcaagtaaaacctctacaaatgtggtatggctgattatgatcgcggccgcattctaccgggtagg
ggaggcgcttttcccaaggcagtctggagcatgcgctttagcagccccgctgggcacttggcgctacacaagtggcctctggcctcgca
cacattccacatccaccggtaggcgccaaccggctccgttctttggtggccccttcgcgccaccttctactcctcccctagtcaggaag
ttcccccccgccccgcagctcgcgtcgtgcaggacgtgacaaatggaagtagcacgtctcactagtctcgtgcagatggacagcaccgc
tgagcaatggaagcgggtaggcctttggggcagcggccaatagcagctttgctccttcgctttctgggctcagaggctgggaaggggtg
ggtccgggggcgggctcaggggcgggctcaggggcggggcgggcgcccgaaggtcctccggaggcccggcattctgcacgcttcaaaag
cgcacgtctgccgcgctgttctcctcttcctcatctccgggcctttcgacctagcgggcagtgagcgcaacgcaattaatgtgagttag
ctcactcattaggcaccccaggctttacactttatgcttccggctcgtatgttgtgtggaattgtgagcggataacaatttcacacagg
aaacagctgccaccatgattgaacaagatggattgcacgcaggttctccggccgcttgggtggagaggctattcggctatgactgggca
caacagacaatcggctgctctgatgccgccgtgttccggctgtcagcgcaggggcgcccggttctttttgtcaagaccgacctgtccgg
tgccctgaatgaactgcaagacgaggcagcgcggctatcgtggctggccacgacgggcgttccttgcgcagctgtgctcgacgttgtca
ctgaagcgggaagggactggctgctattgggcgaagtgccggggcaggatctcctgtcatctcaccttgctcctgccgagaaagtatcc
atcatggctgatgcaatgcggcggctgcatacgcttgatccggctacctgcccattcgaccaccaagcgaaacatcgcatcgagcgagc
acgtactcggatggaagccggtcttgtcgatcaggatgatctggacgaagagcatcaggggctcgcgccagccgaactgttcgccaggc
tcaaggcgagcatgcccgacggcgaggatctcgtcgtgacccatggcgatgcctgcttgccgaatatcatggtggaaaatggccgcttt
tctggattcatcgactgtggccggctgggtgtggcggaccgctatcaggacatagcgttggctacccgtgatattgctgaagagcttgg
cggcgaatgggctgaccgcttcctcgtgctttacggtatcgccgctcccgattcgcagcgcatcgccttctatcgccttcttgacgagt
tcttctgagggggaggctaactgaaacacggaaggagacaataccggaaggaacccgcgctatgacggcaataaaaagacagaataaaa
cgcacggtgttgggtcgtttgttcataaacgcggggttcggtcccagggctggcactctgtcgataccccaccgagaccccattggggc
caatacgcccgcgtttcttccttttccccaccccaccccccaagttcgggtgaaggcccagggctcgcagccaacgtcggggcggcagg
ccctgccatagcctagggataacagggtaatggcgcgggccgcaggaacccctagtgatggagttggccactccctctctgcgcgctcg
ctcgctcactgaggccgggcgaccaaaggtcgcccgacgcccgggctttgcccgggcggcctcagtgagcgagcgagcgcgcagctgcc
tgcaggtggcaaacagctattatgggtattatgggtgacgtcaagcttggcgtaatcatggtcatagctgtttcctgtgtgaaattgtt
atccgctcacaattccacacaacatacgagccggaagcataaagtgtaaagcctggggtgcctaatgagtgagctaactcacattaatt
gcgttgcgctcactgcccgctttccagtcgggaaacctgtcgtgccagctgcattaatgaatcggccaacgcgcggggagaggcggttt
gcgtattgggcgctcttccgcttcctcgctcactgactcgctgcgctcggtcgttcggctgcggcgagcggtatcagctcactcaaagg
cggtaatacggttatccacagaatcaggggataacgcaggaaagaacatgtgagcaaaaggccagcaaaaggccaggaaccgtaaaaag
gccgcgttgctggcgtttttccataggctccgcccccctgacgagcatcacaaaaatcgacgctcaagtcagaggtggcgaaacccgac
aggactataaagataccaggcgtttccccctggaagctccctcgtgcgctctcctgttccgaccctgccgcttaccggatacctgtccg
cctttctcccttcgggaagcgtggcgctttctcatagctcacgctgtaggtatctcagttcggtgtaggtcgttcgctccaagctgggc
tgtgtgcacgaaccccccgttcagcccgaccgctgcgccttatccggtaactatcgtcttgagtccaacccggtaagacacgacttatc
gccactggcagcagccactggtaacaggattagcagagcgaggtatgtaggcggtgctacagagttcttgaagtggtggcctaactacg
gctacactagaagaacagtatttggtatctgcgctctgctgaagccagttaccttcggaaaaagagttggtagctcttgatccggcaaa
caaaccaccgctggtagcggtggtttttttgtttgcaagcagcagattacgcgcagaaaaaaaggatctcaagaagatcctttgatctt
ttctacggggtctgacgctcagtggaacgaaaactcacgttaagggattttggtcatgagattatcaaaaaggatcttcacctagatcc
ttttaaattaaaaatgaagttttaaatcaatctaaagtatatatgagtaaacttggtctgacagttaccaatgcttaatcagtgaggca
cctatctcagcgatctgtctatttcgttcatccatagttgcctgactccccgtgtgtagataactacgatacgggagggcttaccatct
ggccccagtgctgcaatgataccgcgagacccacgctcaccggctccagatttatcagcaataaaccagccagccggaagggccgagcg
cagaagtggtcctgcaactttatccgcctccatccagtctattaattgttgccgggaagctagagtaagtagttcgccagttaatagtt
tgcgcaacgttgttgccattgctacaggcatcgtggtgtcacgctcgtcgtttggtatggcttcattcagctccggttcccaacgatca
aggcgagttacatgatcccccatgttgtgcaaaaaagcggttagctccttcggtcctccgatcgttgtcagaagtaagttggccgcagt
gttatcactcatggttatggcagcactgcataattctcttactgtcatgccatccgtaagatgcttttctgtgactggtgagtactcaa
ccaagtcattctgagaatagtgtatgcggcgaccgagttgctcttgcccggcgtcaatacgggataataccgcgccacatagcagaact
ttaaaagtgctcatcattggaaaacgttcttcggggcgaaaactctcaaggatcttaccgctgttgagatccagttcgatgtaacccac
tcgtgcacccaactgatcttcagcatcttttactttcaccagcgtttctgggtgagcaaaaacaggaaggcaaaatgccgcaaaaaagg
gaataagggcgacacggaaatgttgaatactcatactcttcctttttcaatattattgaagcatttatcagggttattgtctcatgagc
ggatacatatttgaatgtatttagaaaaataaacaaataggggttccgcgcacatttccccgaaaagtgccacctgacgtctaagaaac
cattattatcatgacattaacctataaaaataggcgtatcacgaggccctttcgtctcgcgcgtttcggtgatgacggtgaaaacctct
gacacatgcagctcccggagacggtcacagcttgtctgtaagcggatgccgggagcagacaagcccgtcagggcgcgtcagcgggtgtt
ggcgggtgtcggggctggcttaactatgcggcatcagagcagattgtactgagagtgcaccatatgcggtgtgaaataccgcacagatg
cgtaaggagaaaataccgcatcaggcgccattcgccattcaggctgcgcaactgttgggaagggcgatcggtgcgggcctcttcgctat
tacgccagctggcgaaagggggatgtgctgcaaggcgattaagttgggtaacgccagggttttcccagtcacgacgttgtaaaacgacg
gccagtgaattcacatgt,
Human Elongation Factor Alpha Promoter
>SEQ ID NO: 3
cgtgaggctccggtgcccgtcagtgggcagagcgcacategcccacagtccccgagaagttggggggaggggtcggcaattgaaccggt
gcctagagaaggtggcgcggggtaaactgggaaagtgatgtcgtgtactggctccgcctttttcccgaggggggggagaaccgtatata
agtgcagtagtcgccgtgaacgttctttttcgcaacgggtttgccgccagaacacaggtaagtgccgtgtgtggttcccgcgggcctgg
cctctttacgggttatggcccttgcgtgccttgaattacttccacgcccctggctgcagtacgtgattcttgatcccgagcttcgggtt
ggaagtgggtgggagagttcgaggccttgcgcttaaggagccccttcgcctcgtgcttgagttgaggcctggcctgggcgctggggccg
ccgcgtgcgaatctggtggcaccttcgcgcctgtctcgctgctttcgataagtctctagccatttaaaatttttgatgacctgctgcga
cgctttttttctggcaagatagtcttgtaaatgcgggccaagatctgcacactggtatttcggtttttggggccgcgggcggcgacggg
gcccgtgcgtcccagcgctcatgttcggcgaggcggggcctgcgagcgcggccaccgagaatcggacgggggtagtctcaagctggccg
gcctgctctggtgcctggcctcgcgccgccgtgtatcgccccgccctgggcggcaaggctggcccggtcggcaccagttgcgtgagcgg
aaagatggccgcttcccggccctgctgcagggagctcaaaatggaggacgcggcgctcgggagagcggggggtgagtcacccacacaaa
ggaaaagggcctttccgtcctcagccgtcgcttcatgtgactccacggagtaccgggcgccgtccaggcacctcgattagttctcgagc
ttttggagtacgtcgtctttaggttggggggaggggttttatgcgatggagtttccccacactgagtgggtggagactgaagttaggcc
agcttggcacttgatgtaattctccttggaatttgccctttttgagtttggatcttggttcattctcaagcctcagacagtggttcaaa
gtttttttcttccatttcaggtgtcgtga,
mCherry Coding region
>SEQ ID NO: 4
Atggtgagcaagggcgaggaggataacatggccatcatcaaggagttcatgcgcttcaaggtgcacatggagggctccgtgaacggcca
cgagttcgagatcgagggcgagggcgagggccgcccctacgagggcacccagaccgccaagctgaaggtgaccaagggtggccccctgc
ccttcgcctgggacatcctgtcccctcagttcatgtacggctccaaggcctacgtgaagcaccccgccgacatccccgactacttgaag
ctgtccttccccgagggcttcaagtgggagcgcgtgatgaacttcgaggacggcggcgtggtgaccgtgacccaggactcctccctgca
ggacggcgagttcatctacaaggtgaagctgcgcggcaccaacttcccctccgacggccccgtaatgcagaagaagaccatgggctggg
aggcctcctccgagcggatgtaccccgaggacggcgccctgaagggcgagatcaagcagaggctgaagctgaaggacggcggccactac
gacgctgaggtcaagaccacctacaaggccaagaagcccgtgcagctgcccggcgcctacaacgtcaacatcaagttggacatcacctc
ccacaacgaggactacaccatcgtggaacagtacgaacgcgccgagggccgccactccaccggcggcatggacgagctgtacaagtag.
SV40 polyadenylation signal
>SEQ ID NO: 5
Gatacattgatgagtttggacaaaccacaactagaatgcagtgaaaaaaatgctttatttgtgaaatttgtgatgctattgctttattt
gtaaccattataagctgcaataaacaagttaacaacaacaattgcattcattttatgtttcaggttcagggggaggtgtgggaggtttt
ttaaagcaagtaaaacctctacaaatgtggtatggctgattatgatc,
DNA encoding the wild-type Neomycin phosphotransferase protein
>SEQ ID NO: 6
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
Mouse Phosphoglycerate kinase promoter
>SEQ ID NO: 7
Attctaccgggtaggggaggcgcttttcccaaggcagtctggagcatgcgctttagcagccccgctgggcacttggcgctacacaagtg
gcctctggcctcgcacacattccacatccaccggtaggcgccaaccggctccgttctttggtggccccttegcgccaccttctactcct
cccctagtcaggaagttcccccccgccccgcagctcgcgtcgtgcaggacgtgacaaatggaagtagcacgtctcactagtctcgtgca
gatggacagcaccgctgagcaatggaagcgggtaggcctttggggcagcggccaatagcagctttgctccttcgctttctgggctcaga
ggctgggaaggggtgggtccgggggcgggctcaggggcgggctc,
E. Coli laczya promoter
>SEQ ID NO: 8
Agcgggcagtgagcgcaacgcaattaatgtgagttagctcactcattaggcaccccaggctttacactttatgcttccggctcgtatgt
tgtgtgg,
Herpes Simplex Virus polyadenylation signal
>SEQ ID NO: 9
Gggggaggctaactgaaacacggaaggagacaataccggaaggaacccgcgctatgacggcaataaaaagacagaataaaacgcacggt
gttgggtcgtttgttcataaacgcggggttcggtcccagggctggcactctgtcgataccccaccgagaccccattggggccaatacgc
ccgcgtttcttccttttccccaccccaccccccaagttcgggtgaaggcccagggctcgcagccaacgtcggggggcaggccctgccat
agcc,
ampicillin resistance gene
>SEQ ID NO: 10
ACCCCTATTTGTTTATTTTTCTAAATACATTCAAATATGTATCCGCTCATGAGACAATAACCCTGATAAATGCTTCAATAATATTGAAA
AAGGAAGAGTATGAGTATTCAACATTTCCGTGTCGCCCTTATTCCCTTTTTTGCGGCATTTTGCCTTCCTGTTTTTGCTCACCCAGAAA
CGCTGGTGAAAGTAAAAGATGCTGAAGATCAGTTGGGTGCACGAGTGGGTTACATCGAACTGGATCTCAACAGCGGTAAGATCCTTGAG
AGTTTTCGCCCCGAAGAACGTTTTCCAATGATGAGCACTTTTAAAGTTCTGCTATGTGGCGCGGTATTATCCCGTATTGACGCCGGGCA
AGAGCAACTCGGTCGCCGCATACACTATTCTCAGAATGACTTGGTTGAGTACTCACCAGTCACAGAAAAGCATCTTACGGATGGCATGA
CAGTAAGAGAATTATGCAGTGCTGCCATAACCATGAGTGATAACACTGCGGCCAACTTACTTCTGACAACGATCGGAGGACCGAAGGAG
CTAACCGCTTTTTTGCACAACATGGGGGATCATGTAACTCGCCTTGATCGTTGGGAACCGGAGCTGAATGAAGCCATACCAAACGACGA
GCGTGACACCACGATGCCTGTAGCAATGGCAACAACGTTGCGCAAACTATTAA+NLCTGGCGAACTACTTACTCTAGCTTCCCGGCAAC
AATTAATAGACTGGATGGAGGCGGATAAAGTTGCAGGACCACTTCTGCGCTCGGCCCTTCCGGCTGGCTGGTTTATTGCTGATAAATCT
GGAGCCGGTGAGCGTGGGTCTCGCGGTATCATTGCAGCACTGGGGCCAGATGGTAAGCCCTCCCGTATCGTAGTTATCTACACGACGGG
GAGTCAGGCAACTATGGATGAACGAAATAGACAGATCGCTGAGATAGGTGCCTCACTGATTAAGCATTGGTAACTGTCAGACCAAGTTT
ACTCATATATACTTTAGATTGATTTAAAACTTCATTTTTAATTTAAAAGGATCTAGGTGAAGATCCTTTTTGATAATCTCATGACCAAA
ATCCCTTAACGTGAGTTTTCGTTCCACTGAGCGTCAGACCCCGTAGAAAAGATC,
pUC57 plasmid replication origin
>SEQ ID NO: 11
Cgcgttgctggcgtttttccataggctccgcccccctgacgagcatcacaaaaatcgacgctcaagtcagaggtggcgaaacccgacag
gactataaagataccaggcgtttccccctggaagctccctcgtgcgctctcctgttccgaccctgccgcttaccggatacctgtccgcc
tttctcccttcgggaagcgtggcgctttctcatagctcacgctgtaggtatctcagttcggtgtaggtcgttcgctccaagctgggctg
tgtgcacgaaccccccgttcagcccgaccgctgcgccttatccggtaactatcgtcttgagtccaacccggtaagacacgacttatcgc
cactggcagcagccactggtaacaggattagcagagcgaggtatgtaggcggtgctacagagttcttgaagtggtggcctaactacggc
tacactagaagaacagtatttggtatctgcgctctgctgaagccagttaccttcggaaaaagagttggtagctcttgatccggcaaaca
aaccaccgctggtagcggtggtttttttgtttgcaagcagcagattacgcgcagaaaaaaaggatctcaagaagatccttt,
P614 Neo ORF
>SEQ ID NO: 12
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgaccctgggcgatgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P615 Neo ORF
>SEQ ID NO: 13
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggcggcctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P616 Neo ORF
>SEQ ID NO: 14
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttaacgagttcttctga,
P623 Neo ORF
>SEQ ID NO: 15
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgaccctgggcgatgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttaacgagttcttctga,
P624 Neo ORF
>SEQ ID NO: 16
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcategagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggcggcctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttaacgagttcttctga,
P626 Neo ORF
>SEQ ID NO: 17
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcggcgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttaacgagttcttctga,
APH(6)-Ia amino acid sequence
>SEQ ID NO: 18
(See FIGS. 6A-6B for amino acid sequence),
APH(6)-Ib amino acid sequence
>SEQ ID NO: 19
(See FIGS. 6A-6B for amino acid sequence),
P629 Neo ORF
>SEQ ID NO: 20
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcgggccgctatcaggacatagcgttggctacccgtgatattgctgaagagcttggcggcgaatgggctg
accgcttcctcgtgctttacggtatcgccgctcccgattcgcagcgcatcgccttctatcgccttcttaacgagttcttctga,
P641 Neo ORF
>SEQ ID NO: 21
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctgagttcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P642 Neo ORF
>SEQ ID NO: 22
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcgg
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P643 Neo ORF
>SEQ ID NO: 23
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcgggccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P675 Neo ORF
>SEQ ID NO: 24
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgctatcaggacatagcgttggctacccgtggcattgctgaagagcttggcggcgaatgggctg
accgcttcctcgtgctttacggtatcgccgctcccgattcgcagcgcatcgccttctategccttcttaacgagttcttctga,
P676 Neo ORF
>SEQ ID NO: 25
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgcgatcaggacatagcgttggctacccgtgatattgctgaagagcttggcggcgaatgggctg
accgcttcctcgtgctttacggtatcgccgctcccgattcgcagcgcatcgccttctatcgccttcttaacgagttcttctga,
P677 Neo ORF
>SEQ ID NO: 26
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatgagcgagcacgtactcggatgga
agccggtcttgtcgatcaggatgatctggacgaagagcatcaggggctcgcgccagccgaactgttcgccaggctcaaggcgagcatgc
ccgacggcgaggatctcgtcgtgaccagcggcgatgcctgcttgccgaatatcatggtggaaaatggccgcttttctggattcatcgac
tgtggccggctgggtgtggcggaccgctatcaggacatagcgttggctacccgtgatattgctgaagagcttggcggcgaatgggctga
ccgcttcctcgtgctttacggtatcgccgctcccgattcgcagcgcategccttctategccttcttaacgagttcttctga,
P678 Neo ORF
>SEQ ID NO: 27
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactoggatgg
aagccggtcttgtcgatcaggatgatctggacgaagagcatcaggggctcgcgccagccgaactgttcgccaggctcaaggcgagcatg
cccgacggcgatgatctcgtcgtgacccatggcgatgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcategccttctategccttcttaacgagttcttctga,
P679 Neo ORF
>SEQ ID NO: 28
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtgcccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P680 Neo ORF
>SEQ ID NO: 29
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgcagccaggacatagcgttggctacccgtgatattgctgaagagcttggcggcgaatgggctg
accgcttcctcgtgctttacggtatcgccgctcccgattcgcagcgcategccttctatcgccttcttgacgagttcttctga,
P681 Neo ORF
>SEQ ID NO: 30
Atgattgaacaagatggattgcacgcaggttctccggccgcttgggtggagaggctattcggctatgactgggcacaacagacaatcgg
ctgctctgatgccgccgtgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgctttcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P682 Neo ORF
>SEQ ID NO: 31
Atgattgaacaagatggattgcacgcaggttctccggccgcttgggtggagaggctattcggctatgactgggcacaacagacaatcgg
ctgctctgatgccgccatgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P683 Neo ORF
>SEQ ID NO: 32
Atgattgaacaagatggattgcacgcaggttctccggccgcttgggtggagaggctattcggctatgactgggcacaacagacaatcgg
ctgctctgatgccgccatgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtgcccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P684 Neo ORF
>SEQ ID NO: 33
Atgattgaacaagatggattgcacgcaggttctccggccgcttgggtggagaggctattcggctatgactgggcacaacagacaatcgg
ctgctctgatgccgccatgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactoggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgcagccaggacatagcgttggctacccgtgatattgctgaagagcttggcggcgaatgggctg
accgcttcctcgtgctttacggtatcgccgctcccgattcgcagcgcategccttctatcgccttcttgacgagttcttctga,
P685 Neo ORF
>SEQ ID NO: 34
Atgattgaacaagatggattgcacgcaggttctccggccgcttgggtggagaggctattcggctatgactgggcacaacagacaatcgg
ctgctctgatgccgccatgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtggcggaccgctttcaggacatagcgttggctacccgtgatattgctgaagagcttggcggcgaatgggctg
accgcttcctcgtgctttacggtatcgccgctcccgattcgcagcgcategccttctatcgccttcttgacgagttcttctga,
P686 Neo ORF
>SEQ ID NO: 35
Atgattgaacaagatggattgcacgcaggttctccggccgcttgggtggagaggctattcggctatgactgggcacaacagacaatcgg
ctgctctgatgccgccatgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcategagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgaccagcggcgatgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P687 Neo ORF
>SEQ ID NO: 36
Atgattgaacaagatggattgcacgcaggttctccggccgcttgggtggagaggctattcggctatgactgggcacaacagacaatcgg
ctgctctgatgccgccatgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacategcatcgagcgagcacgtactoggatgg
aagccggtcttgtcgatcaggatgatctggacgaagagcatcaggggctcgcgccagccgaactgttcgccaggctcaaggcgagcatg
cccgacggcgatgatctcgtcgtgacccatggcgatgcctgcttgccgaatatcatggtggaaaatggccgcttttctggattcatcga
ctgtggccggctgggtgtggcggaccgctatcaggacatagcgttggctacccgtgatattgctgaagagcttggcggcgaatgggctg
accgcttcctcgtgctttacggtatcgccgctcccgattcgcagcgcatcgccttctatcgccttcttgacgagttcttctga,
P688 Neo ORF
>SEQ ID NO: 37
atgattgaacaagatggattgcacgcaggttctccggccgcttgggtggagaggctattcggctatgactgggcacaacagacaatcgg
ctgctctgatgccgccatgttccggctgtcagcgcaggggcgcccggttctttttgtcaagaccgacctgtccggtgccctgaatgaac
tgcaagacgaggcagcgcggctatcgtggctggccacgacgggcgttccttgcgcagctgtgctcgacgttgtcactgaagcgggaagg
gactggctgctattgggcgaagtgccggggcaggatctcctgtcatctcaccttgctcctgccgagaaagtatccatcatggctgatgc
aatgcggcggctgcatacgcttgatccggctacctgcccattcgaccaccaagcgaaacatcgcatcgagcgagcacgtactcggatgg
aagccggtcttgtcgatcaggatgatctggacgaagagcatcaggggctcgcgccagccgaactgttcgccaggctcaaggcgagcatg
cccgacggcgaggatctcgtcgtgacccatggcgatgcctgcttgccgaatatcatggtggaaaatggccgcttttctggattcatcga
ctgtggccggctgggtgtgggggccgctatcaggacatagcgttggctacccgtgatattgctgaagagcttggcggcgaatgggctga
ccgcttcctcgtgctttacggtatcgccgctcccgattcgcagcgcatcgccttctatcgccttcttgacgagttcttctga,
P683 (V36M G210A)
>SEQ ID NO: 38
MIEQDGLHAGSPAAWVERLFGYDWAQQTIGCSDAAMFRLSAQGRPVLFVKTDLSGALNELQDEAARLSWLATTGVPCAAVLDVVTEAGR
DWLLLGEVPGQDLLSSHLAPAEKVSIMADAMRRLHTLDPATCPFDHQAKHRIERARTRMEAGLVDQDDLDEEHQGLAPAELFARLKASM
PDGEDLVVTHGDACLPNIMVENGRFSGFIDCARLGVADRYQDIALATRDIAEELGGEWADRFLVLYGIAAPDSQRIAFYRLLDEFF,
P687 (V36M E182D)
>SEQ ID NO: 39
MIEQDGLHAGSPAAWVERLFGYDWAQQTIGCSDAAMFRLSAQGRPVLFVKTDLSGALNELQDEAARLSWLATTGVPCAAVLDVVTEAGR
DWLLLGEVPGQDLLSSHLAPAEKVSIMADAMRRLHTLDPATCPFDHQAKHRIERARTRMEAGLVDQDDLDEEHQGLAPAELFARLKASM
PDGDDLVVTHGDACLPNIMVENGRFSGFIDCGRLGVADRYQDIALATRDIAEELGGEWADRFLVLYGIAAPDSQRIAFYRLLDEFF,
P685 (V36M Y218F)
>SEQ ID NO: 40
MIEQDGLHAGSPAAWVERLFGYDWAQQTIGCSDAAMFRLSAQGRPVLFVKTDLSGALNELQDEAARLSWLATTGVPCAAVLDVVTEAGR
DWLLLGEVPGQDLLSSHLAPAEKVSIMADAMRRLHTLDPATCPFDHQAKHRIERARTRMEAGLVDQDDLDEEHQGLAPAELFARLKASM
PDGEDLVVTHGDACLPNIMVENGRFSGFIDCGRLGVADRFQDIALATRDIAEELGGEWADRFLVLYGIAAPDSQRIAFYRLLDEFF,
P629 (D216G D261N)
>SEQ ID NO: 41
MIEQDGLHAGSPAAWVERLFGYDWAQQTIGCSDAAVFRLSAQGRPVLFVKTDLSGALNELQDEAARLSWLATTGVPCAAVLDVVTEAGR
DWLLLGEVPGQDLLSSHLAPAEKVSIMADAMRRLHTLDPATCPFDHQAKHRIERARTRMEAGLVDQDDLDEEHQGLAPAELFARLKASM
PDGEDLVVTHGDACLPNIMVENGRFSGFIDCGRLGVAGRYQDIALATRDIAEELGGEWADRFLVLYGIAAPDSQRIAFYRLLNEFF,
P684 (V36M Y218S)
>SEQ ID NO: 42
MIEQDGLHAGSPAAWVERLFGYDWAQQTIGCSDAAMFRLSAQGRPVLFVKTDLSGALNELQDEAARLSWLATTGVPCAAVLDVVTEAGR
DWLLLGEVPGQDLLSSHLAPAEKVSIMADAMRRLHTLDPATCPFDHQAKHRIERARTRMEAGLVDQDDLDEEHQGLAPAELFARLKASM
PDGEDLVVTHGDACLPNIMVENGRFSGFIDCGRLGVADRSQDIALATRDIAEELGGEWADRFLVLYGIAAPDSQRIAFYRLLDEFF,
P688 (V36M D216G)
>SEQ ID NO: 43
MIEQDGLHAGSPAAWVERLFGYDWAQQTIGCSDAAMFRLSAQGRPVLFVKTDLSGALNELQDEAARLSWLATTGVPCAAVLDVVTEAGR
DWLLLGEVPGQDLLSSHLAPAEKVSIMADAMRRLHTLDPATCPFDHQAKHRIERARTRMEAGLVDQDDLDEEHQGLAPAELFARLKASM
PDGEDLVVTHGDACLPNIMVENGRFSGFIDCGRLGVAGRYQDIALATRDIAEELGGEWADRFLVLYGIAAPDSQRIAFYRLLDEFF,
>SEQ ID NO: 44
MIEQDGLHAGSPAAWVERLFGYDWAQQTIGCSDAAVFRLSAQGRPVLFVKTDLSGALNELQDEAARLSWLATTGVPCAAVLDVVTEAGR
DWLLLGEVPGQDLLSSHLAPAEKVSIMADAMRRLHTLDPATCPFDHQAKHRIERARTRMEAGLVDQDDLDEEHQGLAPAELFARLKARM
PDGEDLVVTHGDACLPNIMVENGRFSGFIDCGRLGVADRYQDIALATRDIAEELGGEWADRFLVLYGIAAPDSQRIAFYRLLDEFF

>SEQ ID NO:45, APH(6)-Ic amino acid sequence

(See FIGS. 6A-6B for amino acid sequence)

>SEQ ID NO:46, APH(6)-Id amino acid sequence

(See FIGS. 6A-6B for amino acid sequence)

>SEQ ID NO:47, APH(3′)-IIIa amino acid sequence

(See FIGS. 6A-6B for amino acid sequence)

SEQ ID NO:48, APH(3′)-VIIa amino acid sequence

(See FIGS. 6A-6B for amino acid sequence)

SEQ ID NO:49, APH(3′)-VIa amino acid sequence

(See FIGS. 6A-6B for amino acid sequence)

SEQ ID NO:50, APH(3′)-IVa amino acid sequence

(See FIGS. 6A-6B for amino acid sequence)

SEQ ID NO:51, APH(3′)-Ia amino acid sequence

(See FIGS. 6A-6B for amino acid sequence)

SEQ ID NO:52, APH(3′)-Ic amino acid sequence

(See FIGS. 6A-6B for amino acid sequence)

SEQ ID NO:53, APH(3′)-Ib amino acid sequence

(See FIGS. 6A-6B for amino acid sequence)

SEQ ID NO:54, APH(3′)-IIa amino acid sequence

(See FIGS. 6A-6B for amino acid sequence)

SEQ ID NO:55, APH(3′)-Vb amino acid sequence

(See FIGS. 6A-6B for amino acid sequence)

SEQ ID NO:56, APH(3′)-Va amino acid sequence

(See FIGS. 6A-6B for amino acid sequence)

SEQ ID NO:57, APH(3′)-Vc amino acid sequence

(See FIGS. 6A-6B for amino acid sequence)

SEQ ID NO:58, APH(3″)-Ia amino acid sequence

(See FIGS. 6A-6B for amino acid sequence)

SEQ ID NO:59, APH(3″)-Ib amino acid sequence

(See FIGS. 6A-6B for amino acid sequence)

SEQ ID NO:60, APH(2″)-Ia amino acid sequence

(See FIGS. 6A-6B for amino acid sequence)

SEQ ID NO:61, APH(4)-Ib amino acid sequence

(See FIGS. 6A-6B for amino acid sequence)

SEQ ID NO:62, APH(4)-Ib amino acid sequence

(See FIGS. 6A-6B for amino acid sequence)

Particular embodiments of this invention are described herein. Upon reading the foregoing description, variations of the disclosed embodiments may become apparent to individuals working in the art, and it is expected that those skilled artisans may employ such variations as appropriate. Accordingly, it is intended that the invention be practiced otherwise than as specifically described herein, and that the invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context. A number of embodiments of the invention have been described. Nevertheless, it will be understood that various modifications may be made without departing from the spirit and scope of the invention. Accordingly, the descriptions in the Examples section are intended to illustrate but not limit the scope of invention described in the claims.

All references cited herein are incorporated herein by reference in their entirety and for all purposes to the same extent as if each individual publication or patent or patent application was specifically and individually indicated to be incorporated by reference in its entirety for all purposes.

What is claimed:

1. A non-naturally occurring neomycin phosphotransferase (NPT) with neomycin phosphotransferase activity, wherein the non-naturally occurring NPT comprises an amino acid sequence of a wild-type neomycin phosphotransferase with:
(a) amino acid substitutions at amino acid residues corresponding to amino acid residues 36 and 210 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 210 of SEQ ID NO:1 is a substitution to alanine;
(b) amino acid substitutions at amino acid residues corresponding to amino acid residues 36 and 182 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 182 of SEQ ID NO:1 is a substitution to aspartic acid;
(c) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to phenylalanine;
(d) amino acid substitutions at amino acid residues corresponding to amino acid residues 216 and 261 of SEQ ID NO:1, wherein the amino acid substitution at the amino acid residue corresponding to amino acid residue 216 of SEQ ID NO:1 is a substitution to glycine and the amino acid substitution at the amino acid residue corresponding to amino acid residue 261 of SEQ ID NO:1 is a substitution to asparagine;
(e) amino acid substitutions at amino acid residues corresponding to amino acid residues 36 and 218 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 218 of SEQ ID NO:1 is a substitution to serine; or
(f) amino acid substitutions at amino acid residues corresponding to amino acid residues 36 and 216 of SEQ ID NO:1, wherein the amino acid substitution at the amino acid residue corresponding to amino acid residue 36 of SEQ ID NO:1 is a substitution to methionine and the amino acid substitution at the amino acid residue corresponding to amino acid residue 216 of SEQ ID NO:1 is a substitution to glycine.

2. A non-naturally occurring neomycin phosphotransferase (NPT) with neomycin phosphotransferase activity, wherein the non-naturally occurring NPT comprises the amino acid sequence of SEQ ID NO:1 with:
(a) amino acid substitutions at positions 36 and 210 of SEQ ID NO:1, wherein the amino acid substitution at position 36 of SEQ ID NO:1 is a substitution to methionine and the amino acid substitution at position 210 of SEQ ID NO:1 is a substitution to alanine;
(b) amino acid substitutions at positions 36 and 182 of SEQ ID NO:1, wherein the amino acid substitution at position 36 of SEQ ID NO:1 is a substitution to methionine and the amino acid substitution at position 182 of SEQ ID NO:1 is a substitution to aspartic acid;
(c) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to phenylalanine;
(d) amino acid substitutions at positions 216 and 261 of SEQ ID NO:1, wherein the amino acid substitution position 216 of SEQ ID NO:1 is a substitution to glycine and the amino acid substitution at position 261 of SEQ ID NO:1 is a substitution to asparagine;
(e) amino acid substitutions at positions 36 and 218 of SEQ ID NO:1, wherein the amino acid substitution at position 36 of SEQ ID NO:1 is a substitution to methionine and the amino acid substitution at position 218 of SEQ ID NO:1 is a substitution to serine; or
(f) amino acid substitutions at positions 36 and 216 of SEQ ID NO:1, wherein the amino acid substitution at position 36 of SEQ ID NO:1 is a substitution to methionine and the amino acid substitution at position 216 of SEQ ID NO:1 is a substitution to glycine.

3. The NPT of claim 1, wherein the non-naturally occurring NPT has attenuated neomycin phosphotransferase activity as a selectable marker as compared to wild-type NPT.

4. The NPT of claim 1 or 3, wherein the wild-type NPT comprises an amino acid sequence that is at least 80%, at least 90%, or at least 98% identical to SEQ ID NO:1.

5. The NPT of claim 1 or 3, wherein the wild-type NPT comprises an amino acid sequence that is at least 60%, at least 65%, at least 70% or at least 75% identical to SEQ ID NO:1.

6. The NPT of claim 2, wherein the non-naturally occurring NPT has attenuated neomycin phosphotransferase activity as a selectable marker as compared to wild-type NPT comprising the amino acid sequence of SEQ ID NO:1.

7. The NPT of any one of claims 1 to 6, wherein bacterial cells transfected or transformed with a nucleotide sequence encoding the non-naturally occurring NPT exhibit reduced colony formation as assessed by a colony formation assay after 48 hours of growth on plates containing 25 μg/mL, 75 μg/mL or 100 μg/mL kanamycin relative to bacterial cells transfected or transformed with a nucleotide sequence encoding wild-type NPT; and wherein optionally, the bacterial cells are E. coli.

8. The NPT of any one of claims 1 to 6, wherein mammalian cells transfected with an expression vector comprising a nucleotide sequence encoding the non-naturally occurring NPT exhibit reduced colony formation as assessed by a colony formation assay after 2 weeks of growth on tissue culture plates in media containing 500 μg/mL geneticin (G418) relative to mammalian cells transfected with the same expression vector but comprising a nucleotide sequence encoding wild-type NPT; and wherein optionally, the mammalian cells are HEK293 cells, CHO cells, PER.C6 cells, murine NS0 cells, fibrosarcoma HT-1080 cells, murine Sp2/0 cells, BHK cells, or murine C127 cells.

9. The NPT of any one of claims 1 to 6, wherein G418 resistant colonies of mammalian cells transfected with an expression vector comprising a nucleotide sequence encoding the non-naturally occurring NPT are produced at frequencies ranging from 0.001% to 75% relative to G418 resistant colonies of mammalian cells transfected with the same expression vector but comprising a nucleotide sequence encoding wild-type NPT.

10. The NPT of claim 2, wherein the non-naturally occurring NPT comprises the amino acid sequence of SEQ ID NO:38 (V36M, G210A), SEQ ID NO:39 (V36M, E182D), SEQ ID NO:40 (V36M, Y218F), SEQ ID NO:41 (D216G, D261N), SEQ ID NO:42 (V36M, Y218S), or SEQ ID NO:43 (V36M, D216G).

11. A nucleic acid sequence comprising a first nucleotide sequence encoding the non-naturally occurring NPT of any one of claims 1 to 10.

12. The nucleic acid sequence of claim 11, wherein the nucleic acid sequence further comprises a second nucleotide sequence encoding a second protein or a non-coding RNA; and wherein optionally, the second protein is a therapeutic protein.

13. The nucleic acid sequence of claim 11 or 12, wherein the first nucleotide sequence comprises the nucleotide sequence of SEQ ID NO:20, SEQ ID NO:32, SEQ ID NO:33, SEQ ID NO:34, SEQ ID NO: 36, or SEQ ID NO:37.

14. A vector comprising the nucleic acid sequence of any one of claims 11 to 13.

15. An in vitro or ex vivo host cell comprising the non-naturally occurring NPT of any one of claims 1 to 10.

16. An in vitro or ex vivo host cell comprising the nucleic acid sequence of any one of claims 11 to 13, or the vector of claim 14.

17. The cell of claim 16, wherein the nucleic acid sequence is stably integrated into the genome of the host cell.

18. The cell of any one of claims 15 to 17, wherein the host cell further comprises a second nucleic acid sequence encoding a second protein or a non-coding RNA, and wherein the second protein is optionally a therapeutic protein; or wherein optionally, the second nucleic acid sequence encodes a non-coding RNA; and wherein optionally, the non-coding RNA is shRNA, miRNA, antisense RNA, guide RNA for Crispr nucleases, catalytic RNA, ribosomal RNA, or tRNA.

19. The cell of any one of claims 15 to 18, wherein the host cell is a bacterium, yeast cell, mammalian cell, plant cell; optionally wherein the mammalian cell is a human cell.

20. A method for selecting cells with high copy numbers of a transgene and/or high expression levels of a transgene from a population of host cells in which the transgene was introduced, the method comprising:
(a) introducing into a population of host cells a nucleic acid sequence comprising:
(i) a first nucleotide sequence encoding the non-naturally occurring neomycin phosphotransferase (NPT) of any one of claims 1 to 10; and
(ii) a second nucleotide sequence comprising the transgene; and
(b) selecting cells that grow in the presence of a neomycin phosphotransferase substrate from the population of host cells in which the nucleic acid sequence was introduced.

21. The method of claim 20, wherein:
(a) the selected cells comprise a 2 to 1000 times higher copy number of the transgene as compared to the copy number of the transgene in a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene; and/or
(b) the selected cells achieve a 10 to 1000 fold higher level of expression of the transgene as compared to the level of expression of the transgene by a second set of cells following selection of a second population of host cells grown in the presence of a neomycin phosphotransferase substrate, wherein the second population of host cells is transfected or transformed with a nucleic acid sequence comprising a nucleotide sequence encoding wild-type NPT protein and the transgene.

22. The method of claim 20 or 21, wherein the host cells are bacterial cells, yeast cells, mammalian cells, plant cells; optionally wherein the mammalian cells are human cells.

23. The method of claim 20, 21, or 22, wherein the nucleic acid sequence is stably integrated into the genome of the selected cells.

24. The method of any one of claims 20 to 23, wherein the selected cells have a high copy number of the transgene.

25. The method of any one of claims 20 to 24, wherein the selected cells have high level of expression of the transgene.

26. The method of any one of claims 20 to 25, wherein the selected cells have integrated 5 to 100 copies of the transgene into their genomic DNA.

27. The method of any one of claims 20 to 25, wherein the selected cells have integrated 1 to 5 copies of the transgene into their genomic DNA.

28. The method of any one of claims 20 to 27, wherein the transgene comprises a viral gene or growth factor gene, or the transgene encodes a protein or non-coding RNA; wherein optionally, the non-coding RNA is selected from the group consisting of antisense RNA, miRNA, shRNA, long non-coding RNA, catalytic RNA, ribosomal RNA, tRNA or a guide RNA for a CRISPR nuclease; and wherein optionally, the protein is a therapeutic protein or antigen.

29. The method of any one of claims 20 to 28, wherein the neomycin phosphotransferase substrate is neomycin, kanamycin or G418.

30. A method of using a plasmid or transposon comprising a nucleic acid sequence encoding a non-naturally occurring NPT of any one of claims 1 to 10 as a selectable marker, the method comprising:
(a) introducing into a host cell the plasmid or transposon comprising the nucleic acid sequence encoding the non-naturally occurring NPT; and
(b) growing the cell in the presence of a neomycin phosphotransferase substrate.

31. The method of claim 30, wherein the host cell is a bacterium, yeast cell, mammalian cell, plant cell; optionally wherein the mammalian cell is a human cell.

32. The method of claim 30 or 31, wherein the plasmid or transposon further comprises a second nucleotide sequence encoding a protein or a non-coding RNA; wherein optionally, the protein is a viral protein or a therapeutic protein; and wherein optionally, the non-coding RNA is shRNA, miRNA, antisense RNA, guide RNA for Crispr nucleases, catalytic RNA, ribosomal RNA, or tRNA.

33. The method of any one of claims 30 to 32, wherein the neomycin phosphotransferase substrate is neomycin, kanamycin or G418.

34. A method of making host cells comprising a second nucleotide sequence comprising:
(a) introducing into a population of host cells a first nucleic acid sequence comprising (i) a first nucleotide sequence encoding the non-naturally occurring NPT of any one of claims 1 to 10, and (ii) a second nucleotide sequence comprising a transgene encoding a second protein or a non-coding RNA; wherein optionally, the second protein is a therapeutic protein or an antigen, or optionally the non-coding region is shRNA, miRNA, antisense RNA, guide RNA for Crispr nucleases, catalytic RNA, ribosomal RNA or tRNA;
(b) growing the population of host cells in the presence of a neomycin phosphotransferase substrate to produce colonies; and
(c) selecting a colony of cells that grows in the presence of the neomycin phosphotransferase substrate.

35. A method of making host cells comprising a second nucleotide sequence comprising:
(a) co-introducing into a population of host cells (i) a first nucleic acid sequence comprising a first nucleotide sequence encoding the non-naturally occurring NPT of any one of claims 1 to 10, and (ii) a second nucleic acid sequence comprising a transgene encoding a second protein or a non-coding RNA; wherein optionally, the second protein is a therapeutic protein or an antigen, or optionally the non-coding region is shRNA, miRNA, antisense RNA, guide RNA for Crispr nucleases, catalytic RNA, ribosomal RNA or tRNA;
(b) growing the population of host cells in the presence of a neomycin phosphotransferase substrate to produce colonies; and
(c) selecting a colony of cells that grows in the presence of the neomycin phosphotransferase substrate.

36. A method of making host cells comprising a second nucleotide sequence comprising:
(a) growing a population of hosts cells in the presence of a neomycin phosphotransferase substrate to produce colonies, wherein the population of host cells comprises (i) a first nucleic acid sequence comprising a first nucleotide sequence encoding the non-naturally occurring NPT of any one of claims 1 to 10, and (ii) a second nucleic acid sequence comprising a transgene encoding a second protein or a non-coding RNA; wherein optionally, the second protein is a therapeutic protein or an antigen, or optionally the non-coding region is shRNA, miRNA, antisense RNA, guide RNA for Crispr nucleases, catalytic RNA, ribosomal RNA or tRNA; and
(b) selecting a colony of cells that grows in the presence of the neomycin phosphotransferase substrate.

37. A method of making host cells comprising a second nucleotide sequence comprising:
(a) growing a population of hosts cells in the presence of a neomycin phosphotransferase substrate to produce colonies, wherein the population of host cells comprises (i) a first nucleic acid sequence comprising a first nucleotide sequence encoding the non-naturally occurring NPT of any one claims 1 to 10, and (ii) a second nucleic acid sequence comprising a transgene encoding a second protein or a non-coding RNA; wherein optionally, the second protein is a therapeutic protein or an antigen, or optionally the non-coding region is shRNA, miRNA, antisense RNA, guide RNA for Crispr nucleases, catalytic RNA, ribosomal RNA or tRNA; and
(b) selecting a colony of cells that grows in the presence of the neomycin phosphotransferase substrate.

38. The method of any one of claims 34 to 37, wherein the host cells are mammalian cells; optionally wherein the mammalian cells are human cells.

39. The method of claim 38, wherein the mammalian cells are HEK293 cells, CHO cells, PER.C6 cells, murine NS0 cells, fibrosarcoma HT-1080 cells, murine Sp2/0 cells, BHK cells, or murine C127 cells.

40. The method of any one of claims 34 to 39, which further comprises culturing the selected colony of cells.

41. The method of any one of claims 34 to 39, wherein the neomycin phosphotransferase substrate is neomycin, kanamycin, or G418.

42. Host cells produced by the method of any one of claims 34 to 41.

43. A method for manufacturing a stable cell line expressing a therapeutic protein or enzyme comprising:
(a) introducing one or more nucleic acid sequences into a population of host cells, wherein the one or more nucleic acid sequences comprise: (i) a first nucleic acid sequence encoding the non-naturally occurring neomycin phosphotransferase (NPT) of any one of claims 1 to 10; and (ii) a second nucleic acid sequence encoding the therapeutic protein or enzyme;
(b) selecting a cell from the population of cells of step (a) that grows in the presence of G418; and
(c) culturing the selected cell to produce a stable cell line expressing the therapeutic protein or enzyme.

44. The method of claim 43, wherein the stable cell line expresses the therapeutic protein or enzyme, optionally wherein the therapeutic protein is an antibody or antibody fragment.

45. A stable cell line produced by the method of claim 43 or 44.

46. A method of making a virus producer cell line comprising:
(a) introducing one or more nucleic acid sequences into a population of host cells, wherein the one or more nucleic acid sequences comprise: (i) a first nucleic acid sequence encoding the non-naturally occurring neomycin phosphotransferase (NPT) of any one of claims 1 to 10; and (ii) a second nucleic acid sequence encoding one or more viral proteins, wherein the one or more viral proteins include a capsid protein, an envelope protein, a viral protein necessary for replication, or a combination thereof;
(b) selecting a cell from the population of cells that grows in the presence in of a neomycin phosphotransferase substrate; and
(c) propagating the selected cell to produce a virus producer cell line.

47. The method of claim 46, wherein the one or more viral proteins include an AAV capsid protein; an AAV capsid protein and AAV rep protein; an envelope protein; adenovirus E1 region proteins required for adenovirus replication; a retroviral envelope protein; a retroviral gag protein; a retroviral reverse transcriptase; or a retroviral envelope protein, gag protein and reverse transcriptase.

48. A virus producer cell line made by the method of claim 46 or 47.

49. A virus producer cell line comprising one or more nucleic acid sequences, wherein the one or more nucleic acid sequences comprise:
(a) a first nucleic acid sequence encoding the non-naturally occurring neomycin phosphotransferase (NPT) of any one of claims 1 to 10; and
(b) a second nucleic acid sequence encoding one or more viral proteins, wherein the one or more viral proteins include a capsid protein, an envelope protein, a viral protein necessary for replication, or a combination thereof.

50. The virus producer cell line of claim 49, wherein the one or more viral proteins includes an AAV capsid protein; an AAV capsid protein and AAV rep protein; an envelope protein;
adenovirus E1 region proteins required for adenovirus replication; a retroviral envelope protein;
a retroviral gag protein; a retroviral reverse transcriptase; or a retroviral envelope protein, gag protein and reverse transcriptase.

51. A method for manufacturing a cell line expressing an antigen comprising:
(a) introducing one or more nucleic acid sequences into a population of host cells, wherein the one or more nucleic acid sequences comprise: (i) a first nucleic acid sequence encoding the non-naturally occurring neomycin phosphotransferase (NPT) of any one of claims 1 to 10; and (ii) a second nucleic acid sequence encoding an antigen; wherein optionally, the antigen is a viral antigen, a bacterial antigen, a fungal antigen, or a cancer antigen;
(b) selecting a cell from the population of cells of step (a) that grows in the presence of G418; and
(c) culturing the selected cell to produce a cell line expressing the antigen.

52. An antigen producing cell line made by the method of claim 51.

53. The method of claim 43, 44, 46, 47 or 51, wherein the cell line is a mammalian cell line; optionally wherein the mammalian cell line is a human cell line.

54. The method of claim 43, 44, 46, 47, or 51, wherein the cell line is a CHO, PER.C6, murine NS0, HEK293, fibrosarcoma HT-1080, murine Sp2/0, BHK, or murine C127 cell line.

55. An antigen producing cell line comprising one or more nucleic acid sequences, wherein the one or more nucleic acid sequences comprise:
(a) a first nucleic acid sequence encoding the non-naturally occurring neomycin phosphotransferase (NPT) of any one of claims 1 to 10; and
(b) a second nucleic acid sequence encoding one or more antigens; wherein optionally, the one or more antigens is a viral antigen, a bacterial antigen, a fungal antigen or a cancer antigen.

56. The cell line of claim 45, 48, 49, 50, 52 or 55, wherein the cell line is a mammalian cell line; optionally wherein the mammalian cell line is a human cell line.

57. The cell line of claim 45, 48, 49, 50, 52, or 55, wherein the cell line is a CHO, PER.C6, murine NS0, HEK293, fibrosarcoma HT-1080, murine Sp2/0, BHK, or murine C127 cell line.

58. A selectable marker means for conferring resistance to kanamycin when introduced into a bacterial cell, and to G418 when introduced into a mammalian cell; wherein optionally, the selectable marker means comprises a nucleic acid sequence of SEQ ID NO:20; a nucleic acid sequence of SEQ ID NO:32; a nucleic acid sequence of SEQ ID NO:33; a nucleic acid sequence of SEQ ID NO:34; a nucleic acid sequence of SEQ ID NO:36; or a nucleic acid sequence of SEQ ID NO:37.

59. A method for manufacturing a producer cell line comprising:
(a) transforming a bacterial or mammalian cell with an expression vector comprising a nucleic acid sequence encoding one or more viral proteins and a means for growing in the presence of kanamycin if the transformed cell is a bacterial cell and for growing in the presence of G418 if the transformed cell is a mammalian cell to make a transformed cell; and
(b) culturing the transformed cell in the presence of kanamycin or G418 to obtain a producer cell line, wherein the producer cell line expresses one or more viral proteins from AAV, adenovirus, retrovirus, lentivirus, herpes simplex virus, vaccinia virus or baculovirus.

60. A method for selecting a cell with stable chromosomal integration of an exogenous nucleic acid sequence comprising:
(a) transforming a population of eukaryotic cells with an exogenous nucleic acid sequence comprising a means for growing in the presence of G418;
(b) culturing the population of transformed cells in the presence of G418 to produce colonies of transformed cells capable of growing in the presence of G418; and
(c) selecting a cell from a colony produced in step (b) to obtain a cell with a stable chromosomal integration of the exogenous nucleic acid; wherein optionally, the exogenous nucleic acid sequence further comprises a transgene, and the selected cell expresses the transgene; or the exogenous nucleic acid sequence disrupts expression of a gene endogenous to the selected cell.

61. A method for selecting a mammalian cell with a stable episome comprising:
(a) transforming a population of mammalian cells with a plasmid comprising a means for growing in the presence of G418;
(b) culturing the population of transformed cells in the presence of G418 to produce colonies of transformed cells capable of growing in the presence of G418; and
(c) selecting a cell from a colony produced in step (b) to obtain a cell with a stable episome comprising the plasmid; wherein optionally, the plasmid further comprises an EBNA1 OriP nucleic acid sequence and the selected cell expresses EBNA1.

62. A method for selecting a mammalian cell transiently expressing a transgene comprising:
(a) introducing into a population of mammalian cells a nucleic acid encoding a transgene and a means for growing in the presence of G418;
(b) culturing the population of mammalian cells in the presence of G418 for 48-72 hours; and
(c) selecting a mammalian cell from the cultured population of mammalian cells that grows in the presence of G418, wherein the selected mammalian cell transiently expresses the transgene; wherein optionally, the transgene comprises nucleic acid sequences encoding a Crispr endonuclease or a Crispr guide RNA.

63. The method of claim 59 to 62, wherein the means is nucleotide sequence encoding a non-naturally occurring neomycin phosphotransferase comprising the amino acid sequence selected from the group of SEQ ID NO: 38, 39, 40, 41, 42 and 43.
